(19) United States
(12) Patent Application Publication
(10) Pub. No.: US 2011/0092526 A1
(43) Pub. Date: Apr. 21, 2011

(54) Adamantyl Iminocarbonyl-Substituted Pyrimidines As Inhibitors Of 11-Beta-HSD1 826

(75) Inventors: Stuart Norman Lile Bennett, Cheshire (GB); Frederick Woolf Goldberg, Cheshire (GB); Andrew Leach, Cheshire (GB); Paul Robert Owen Whittamore, Cheshire (GB); Pernilla Sörme, Molndal (SE)

(73) Assignee: ASTRAZENECA AB, Sodertalje (SE)

(21) Appl. No.: 12/907,056

(22) Filed: Oct. 19, 2010

Related U.S. Application Data
(60) Provisional application No. 61/253,191, filed on Oct. 20, 2009.

(51) Int. Cl. A61K 31/506; C07D 239/28; C07D 239/38; C07D 239/47; A61K 31/505; A61P 3/00

(52) U.S. Cl. 514/272; 544/332; 544/318; 544/301; 514/275; 514/269; 514/274

(57) ABSTRACT

A compound of formula (I):

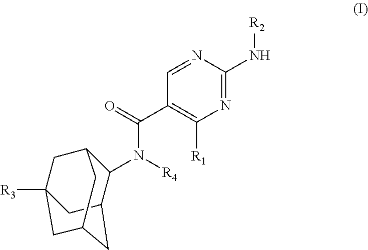

and pharmaceutically-acceptable salts thereof, wherein the variable groups are defined within; their use in the inhibition of 11βHSD1, processes for making them and pharmaceutical compositions comprising them are also described herein.

This invention relates to chemical compounds, or pharmaceutically-acceptable salts thereof. These compounds possess human 11-β-hydroxysteroid dehydrogenase type 1 enzyme (11βHSD1) inhibitory activity and accordingly have value in the treatment of disease states including metabolic syndrome and are useful in methods of treatment of a warm-blooded animal, such as man. The invention also relates to processes for the manufacture of said compounds, to pharmaceutical compositions containing them and to their use in the manufacture of medicaments to inhibit 11βHSD1 in a warm-blooded animal, such as man.

Glucocorticoids (cortisol in man, corticosterone in rodents) are counter regulatory hormones i.e. they oppose the actions of insulin (Dallman M F, Strack A M, Akana S F et al. 1993; Front Neuroendocrinol 14, 303-347). They regulate the expression of hepatic enzymes involved in gluconeogenesis and increase substrate supply by releasing glycerol from adipose tissue (increased lipolysis) and amino acids from muscle (decreased protein synthesis and increased protein degradation). Glucocorticoids are also important in the differentiation of pre-adipocytes into mature adipocytes which are able to store triglycerides (Bujalska I J et al. 1999; Endocrinology 140, 3188-3196). This may be critical in disease states where glucocorticoids induced by “stress” are associated with central obesity which itself is a strong risk factor for type 2 diabetes, hypertension and cardiovascular disease (Bjorntorp P & Rosmond R 2000; Int. J. Obesity 24, S80-S85).

It is now well established that glucocorticoid activity is controlled not simply by secretion of cortisol but also at the tissue level by intracellular interconversion of active cortisol and inactive cortisone by the 11-beta hydroxysteroid dehydrogenases, 11βHSD1 (which activates cortisone) and 11βHSD2 (which inactivates cortisol) (Sandeep T C & Walker B R 2001 Trends in Endocrinol & Metab. 12, 446-453). That this mechanism may be important in man was initially shown using carbenoxolone (an anti-ulcer drug which inhibits both 11βHSD1 and 2) treatment which (Walker B R et al. 1995; J. Clin. Endocrinol. Metab. 80, 3155-3159) leads to increased insulin sensitivity indicating that 11βHSD1 may well be regulating the effects of insulin by decreasing tissue levels of active glucocorticoids (Walker B R et al. 1995; J. Clin. Endocrinol. Metab. 80, 3155-3159).

Clinically, Cushing's syndrome is associated with cortisol excess which in turn is associated with glucose intolerance, central obesity (caused by stimulation of pre-adipocyte differentiation in this depot), dyslipidaemia and hypertension. Cushing's syndrome shows a number of clear parallels with metabolic syndrome. Even though the metabolic syndrome is not generally associated with excess circulating cortisol levels (Jessop D S et al. 2001; J. Clin. Endocrinol. Metab. 86, 4109-4114) abnormally high 11βHSD1 activity within tissues would be expected to have the same effect. In obese men it was shown that despite having similar or lower plasma cortisol levels than lean controls, 11βHSD1 activity in subcutaneous fat was greatly enhanced (Rask E et al. 2001; J. Clin. Endocrinol. Metab. 1418-1421). Furthermore, the central fat, associated with the metabolic syndrome expresses much higher levels of 11βHSD1 activity than subcutaneous fat (Bujalska I J et al. 1997; Lancet 349, 1210-1213). Thus there appears to be a link between glucocorticoids, 11βHSD1 and the metabolic syndrome.

11βHSD1 knock-out mice show attenuated glucocorticoid-induced activation of gluconeogenic enzymes in response to fasting and lower plasma glucose levels in response to stress or obesity (Kotelevtsev Y et al. 1997; Proc. Natl. Acad. Sci. USA 94, 14924-14929) indicating the utility of inhibition of 11βHSD1 in lowering of plasma glucose and hepatic glucose output in type 2 diabetes. Furthermore, these mice express an anti-atherogenic lipoprotein profile, having low triglycerides, increased HDL cholesterol and increased apo-lipoprotein AI levels. (Morton N M et al. 2001; J. Biol. Chem. 276, 41293-41300). This phenotype is due to an increased hepatic expression of enzymes of fat catabolism and PPARα. Again this indicates the utility of 11βHSD1 inhibition in treatment of the dyslipidaemia of the metabolic syndrome.

The most convincing demonstration of a link between the metabolic syndrome and 11βHSD1 comes from recent studies of transgenic mice over-expressing 11βHSD1 (Masuzaki H et al. 2001; Science 294, 2166-2170). When expressed under the control of an adipose specific promoter, 11βHSD1 transgenic mice have high adipose levels of corticosterone, central obesity, insulin resistant diabetes, hyperlipidaemia and hyperphagia. Most importantly, the increased levels of 11βHSD1 activity in the fat of these mice are similar to those seen in obese subjects. Hepatic 11βHSD1 activity and plasma corticosterone levels were normal, however, hepatic portal vein levels of corticosterone were increased 3 fold and it is thought that this is the cause of the metabolic effects in liver.

Overall it is now clear that the complete metabolic syndrome can be mimicked in mice simply by overexpressing 11βHSD1 in fat alone at levels similar to those in obese man.

11βHSD1 tissue distribution is widespread and overlapping with that of the glucocorticoid receptor. Thus, 11βHSD1 inhibition could potentially oppose the effects of glucocorticoids in a number of physiological/pathological roles. 11βHSD1 is present in human skeletal muscle and glucocorticoid opposition to the anabolic effects of insulin on protein turnover and glucose metabolism are well documented (Whorwood C B et al. 2001; J. Clin. Endocrinol. Metab. 86, 2296-2308). Skeletal muscle must therefore be an important target for 11βHSD1 based therapy.

Glucocorticoids also decrease insulin secretion and this could exacerbate the effects of glucocorticoid induced insulin resistance. Pancreatic islets express 11βHSD1 and carbenoxolone can inhibit the effects of 11-dehydrocorticosterone on insulin release (Davani B et al. 2000; J. Biol. Chem. 275, 34841-34844). Thus in treatment of diabetes 11βHSD1 inhibitors may not only act at the tissue level on insulin resistance but also increase insulin secretion itself.

Skeletal development and bone function is also regulated by glucocorticoid action. 11βHSD1 is present in human bone osteoclasts and osteoblasts and treatment of healthy volunteers with carbenoxolone showed a decrease in bone resorption markers with no change in bone formation markers (Cooper M S et al 2000; Bone 27, 375-381) Inhibition of 11βHSD1 activity in bone could be used as a protective mechanism in treatment of osteoporosis.

Glucocorticoids may also be involved in diseases of the eye such as glaucoma. 11βHSD1 has been shown to affect intraocular pressure in man and inhibition of 11βHSD1 may be expected to alleviate the increased intraocular pressure associated with glaucoma (Rauz S et al. 2001; Investigative Opthalmology & Visual Science 42, 2037-2042).

There appears to be a convincing link between 11βHSD1 and the metabolic syndrome both in rodents and in humans. Evidence suggests that a drug which specifically inhibits 11βHSD1 in type 2 obese diabetic patients will lower blood glucose by reducing hepatic gluconeogenesis, reduce central obesity, improve the atherogenic lipoprotein phenotype, lower blood pressure and reduce insulin resistance. Insulin effects in muscle will be enhanced and insulin secretion from the beta cells of the islet may also be increased.

Currently there are two main recognised definitions of metabolic syndrome.

1) The Adult Treatment Panel (ATP III 2001 JMA) definition of metabolic syndrome indicates that it is present if the patient has three or more of the following symptoms:
Waist measuring at least 40 inches (102 cm) for men, 35 inches (88 cm) for women;
Serum triglyceride levels of at least 150 mg/dl (1.69 mmol/l);
HDL cholesterol levels of less than 40 mg/dl (1.04 mmol/l) in men, less than 50 mg/dl (1.29 mmol/l) in women;
Blood pressure of at least 135/80 mm Hg; and/or Blood sugar (serum glucose) of at least 110 mg/dl (6.1 mmol/l).
2) The WHO consultation has recommended the following definition which does not imply causal relationships and is suggested as a working definition to be improved upon in due course:
The patient has at least one of the following conditions: glucose intolerance, impaired glucose tolerance (IGT) or diabetes mellitus and/or insulin resistance; together with two or more of the following:

### Raised Arterial Pressure;

Raised plasma triglycerides

### Central Obesity

### Microalbuminuria

We have found that the compounds defined in the present invention, or a pharmaceutically-acceptable salt thereof, are effective 11βHSD1 inhibitors, and accordingly have value in the treatment of disease states associated with metabolic syndrome.

Accordingly, in a 1st embodiment, there is provided a compound of formula (I) or a pharmaceutically-acceptable salt thereof:

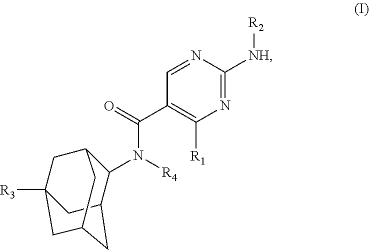

wherein:

R₁ is trifluoromethyl, propyl, cyclobutyl, cyclopentyl, ethoxy, propoxy, cyclobutoxy, pyrrolidinyl, or pyrazolyl;

R₂ is tetrahydrofuranyl, 1-acetylpiperidyl, oxopiperidyl, dioxothiolanyl, hydroxycyclopentyl, hydroxyethyl, hydroxypropyl, 2-(dimethylamino)-2-oxo-ethyl, methoxyethyl, oxopyrrolidinyl, 1-acetylpyrrolidinyl, oxetanyl, hydroxydimethyl-ethyl, 1,1-dioxothianyl, tetrahydropyranyl, (tetrahydrofuranyl)methylenyl, (oxopyrrolidinyl)methylenyl, (methyl-oxo-pyrrolidinyl)methylenyl or pyrrolidinyl;

R₃ is hydroxy, carboxy, carbamoyl, or methylsulphonyl;

R₄ is hydrogen or methyl;

with the proviso that said compound is not:
4-cyclobutyl-N-[(2r,5s)-5-hydroxyadamantan-2-yl]-2-[(2-hydroxy-1,1-dimethylethyl)amino]pyrimidine-5-carboxamide;
4-cyclobutyl-N-[(2r,5s)-5-hydroxyadamantan-2-yl]-2-[(2-hydroxyethyl)amino]pyrimidine-5-carboxamide;
4-cyclobutyl-N-[(2r,5s)-5-hydroxyadamantan-2-yl]-2-[(2-hydroxy-2-methylpropyl)amino]pyrimidine-5-carboxamide;
4-cyclobutyl-N-[(2r,5s)-5-hydroxyadamantan-2-yl]-2-(oxetan-3-ylamino)pyrimidine-5-carboxamide;
4-cyclobutyl-2-[(1,1-dioxidotetrahydro-2H-thiopyran-4-yl)amino]-N-[(2r,5s)-5-hydroxyadamantan-2-yl]pyrimidine-5-carboxamide;
4-cyclopentyl-N-[(2r,5s)-5-hydroxyadamantan-2-yl]-2-oxetan-3-ylamino)pyrimidine-5-carboxamide;
4-cyclopentyl-2-[(1,1-dioxothian-4-yl)amino]-N-[(2r,5s)-5-hydroxyadamantan-2-yl]pyrimidine-5-carboxamide;
4-cyclopentyl-N-[(2r,5s)-5-hydroxyadamantan-2-yl]-2-[(2-hydroxy-2-methylpropyl)amino]pyrimidine-5-carboxamide;
4-cyclopentyl-N-[(2r,5s)-5-hydroxyadamantan-2-yl]-2-(2-hydroxyethylamino)pyrimidine-5-carboxamide;
4-cyclopentyl-N-[(2r,5s)-5-hydroxyadamantan-2-yl]-2-[(1-hydroxy-2-methylpropan-2-yl)amino]pyrimidine-5-carboxamide;
4-cyclopentyl-N-[(2r,5s)-5-hydroxyadamantan-2-yl]-2-(oxan-4-ylamino)pyrimidine-5-carboxamide;
4-cyclopentyl-N-[(2r,5s)-5-hydroxyadamantan-2-yl]-2-[[(3R)-oxolan-3-yl]amino]pyrimidine-5-carboxamide;
4-cyclopentyl-N-[(2r,5s)-5-hydroxyadamantan-2-yl]-2-[[(3S)-oxolan-3-yl]amino]pyrimidine-5-carboxamide;
4-ethoxy-N-[(2r,5s)-5-hydroxytricyclo[3.3.1.1^{3,7}]dec-2-yl]-2-(oxetan-3-ylamino)pyrimidine-5-carboxamide; or
N-[(2r,5s)-5-hydroxytricyclo[3.3.1.1^{3,7}]dec-2-yl]-4-(1-methylethoxy)-2-(oxetan-3-ylamino)pyrimidine-5-carboxamide.

Specific embodiments or aspects of variable groups or terms are as follows. Such values, aspects or embodiments may be used where appropriate to combine with any of the values, definitions, claims, aspects, embodiments, particular embodiments or embodiments of the invention defined hereinbefore or hereinafter. In particular, each may be used as an individual limitation on the broadest definition of formula (I).

It will be understood that when formula I compounds contain a chiral centre, the compounds of the invention may exist in, and be isolated in, optically active or racemic form. The invention includes any optically active or racemic form of a compound of formula I which act as a 11βHSD1 inhibitor. The synthesis of optically active forms may be carried out by standard techniques of organic chemistry well known in the art, for example by, resolution of a racemic mixture, by chiral chromatography, synthesis from optically active starting materials or by asymmetric synthesis.

It will also be understood that the compounds of the formula I may exhibit the phenomenon of tautomerism, the present invention includes any tautomeric form of a compound of formula I which is a 11βHSD1 inhibitor.

It will also be understood that in so far as compounds of the present invention exist as solvates, and in particular hydrates, these are included as part of the present invention.

The invention also relates to in vivo hydrolysable esters of a compound of the formula (I). In vivo hydrolysable esters are those esters that are broken down in the animal body to produce the parent carboxylic acid.

In one embodiment of the invention are provided compounds of formula (I). In an alternative embodiment are provided pharmaceutically-acceptable salts of compounds of formula (I).

It is also to be understood that unspecified terms for alkyls such as “butyl” include both the straight chain and branched chain groups such as n-butyl, iso-butyl and tert-butyl, unless (otherwise) specified. However, when a specific term such as “n-butyl” is used, it is specific for the straight chain or “normal” butyl group, and branched chain isomers such as “t-butyl” not being intended.

It is further to be understood that unspecified terms for alkoxyls such as “propoxy” include both the straight chain and branched chain groups such as n-propoxy and iso-propoxy, unless (otherwise) specified. However, when a specific term such as “n-propoxy” is used, it is specific for the straight chain or “normal” propoxy group, and branched chain isomers such as “iso-propoxy” not being intended.

### DEFINITIONS

“methylenyl” designates —CH₂—;
“cyclobutyl” designates

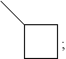

“cyclopentyl” designates

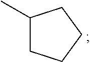

“hydroxycyclopentyl” designates

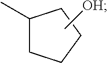

“cyclobutoxy” designates

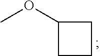

“pyrrolidinyl” designates

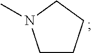

“pyrazolyl” designates

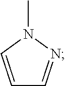

“tetrahydrofuranyl” designates

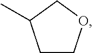

having the O-atom in 2- or 3-position;
“1-acetylpiperidyl” designates

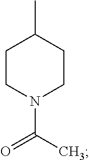

“oxopiperidyl” designates

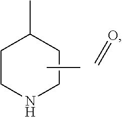

having the oxo group in 2, 3, 5 or 6 position;
“dioxothiolanyl” designates

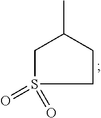

“2-(dimethylamino)-2-oxo-ethyl”

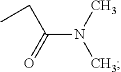

“oxopyrrolidinyl” designates

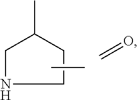

having the oxo group in 2, 4 or 5 position;
“1-acetylpyrrolidinyl” designates

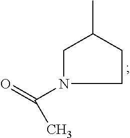

“oxetanyl” designates

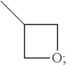

“1,1-dioxothianyl” designates

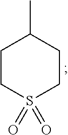

“tetrahydropyranyl” designates

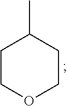

“(tetrahydrofuranyl)methylenyl” designates

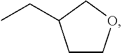

having the O-atom in 2- or 3-position;
“(oxopyrrolidinyl)methylenyl” designates

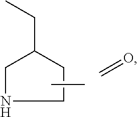

having the oxo group in 2, 4 or 5 position;
“(1-methyl-oxopyrrolidinyl)methylenyl” designates

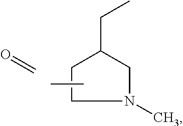

having the oxo group in 2, 4 or 5 position;
“carbamoyl” designates

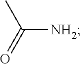

“methylsulphonyl” designates

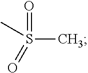

### Rᵢ

a) is in one aspect selected from trifluoromethyl, propyl, cyclobutyl, cyclopentyl, ethoxy, propoxy and cyclobutoxy;

b) is in one aspect selected from trifluoromethyl, propyl, cyclobutyl, cyclopentyl, ethoxy, and propoxy;

c) is in one aspect selected from propyl, cyclobutyl, cyclopentyl, ethoxy, propoxy and cyclobutoxy;

d) is in one aspect selected from cyclobutyl, cyclopentyl, ethoxy, propoxy and cyclobutoxy;

e) is in one aspect selected from trifluoromethyl, propyl, cyclobutyl, cyclopentyl, propoxy and cyclobutoxy.

f) is in one aspect selected from cyclopentyl and cyclobutoxy;

### R₂

a) is in one aspect selected from tetrahydrofuranyl, 1-acetylpiperidyl, oxopiperidyl, dioxothiolanyl, hydroxycyclopentyl, hydroxyethyl, hydroxypropyl, 2-(dimethylamino)-2-oxo-ethyl, methoxyethyl, oxopyrrolidinyl, 1-acetylpyrrolidinyl, oxetanyl, hydroxydimethyl-ethyl, 1,1-dioxothianyl, tetrahydropyranyl or pyrrolidinyl;

b) is in one aspect selected from tetrahydrofuranyl, 1-acetylpiperidyl, oxopiperidyl, hydroxycyclopentyl, hydroxyethyl, hydroxypropyl, 2-(dimethylamino)-2-oxo-ethyl, methoxyethyl, oxopyrrolidinyl, 1-acetylpyrrolidinyl, oxetanyl, hydroxydimethyl-ethyl, tetrahydropyranyl, (tetrahydrofuranyl)methylenyl, (oxopyrrolidinyl)methylenyl, (methyl-oxo-pyrrolidinyl)methylenyl or pyrrolidinyl;

c) is in one aspect selected from tetrahydrofuranyl, dioxothiolanyl, hydroxycyclopentyl, hydroxyethyl, hydroxypropyl, 2-(dimethylamino)-2-oxo-ethyl, methoxyethyl, oxetanyl, hydroxydimethyl-ethyl, 1,1-dioxothianyl, or (tetrahydrofuranyl)methylenyl;

d) is in one aspect selected from tetrahydrofuranyl, (tetrahydrofuranyl)methylenyl, (oxopyrrolidinyl)methylenyl or (methyl-oxo-pyrrolidinyl)methylenyl;

e) is in one aspect tetrahydrofuranyl;

### R₃

a) is in one aspect selected from hydroxy, carbamoyl and methylsulphonyl;

b) is in one aspect selected from hydroxyl and carbamoyl;

c) is in one aspect selected from hydroxyl and methylsulphonyl;

d) is in one aspect hydroxyl;

### R₄

a) is in one aspect hydrogen;

b) is in one aspect methyl.

In a 2^{nd} embodiment of formula (I), formula (I) is defined as being

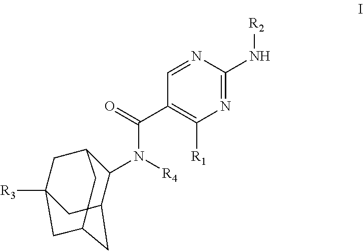

wherein:

R₁ is trifluoromethyl, propyl, cyclobutyl, cyclopentyl, ethoxy, propoxy, or cyclobutoxy;

R₂ is tetrahydrofuranyl, 1-acetylpiperidyl, oxopiperidyl, dioxothiolanyl, hydroxycyclopentyl, hydroxyethyl, hydroxypropyl, 2-(dimethylamino)-2-oxo-ethyl, methoxyethyl, oxopyrrolidinyl, 1-acetylpyrrolidinyl, oxetanyl, hydroxydimethyl-ethyl, 1,1-dioxothianyl, tetrahydropyranyl, (tetrahydrofuranyl)methylenyl, (oxopyrrolidinyl)methylenyl, (methyl-oxo-pyrrolidinyl)methylenyl or pyrrolidinyl;

R₃ is hydroxy or carbamoyl;

R₄ is hydrogen or methyl;

with the proviso that said compound is not:
4-cyclobutyl-N-[(2r,5s)-5-hydroxyadamantan-2-yl]-2-[(2-hydroxy-1,1-dimethylethyl)amino]pyrimidine-5-carboxamide;
4-cyclobutyl-N-[(2r,5s)-5-hydroxyadamantan-2-yl]-2-[(2-hydroxyethyl)amino]pyrimidine-5-carboxamide;
4-cyclobutyl-N-[(2r,5s)-5-hydroxyadamantan-2-yl]-2-[(2-hydroxy-2-methylpropyl)amino]pyrimidine-5-carboxamide;
4-cyclobutyl-N-[(2r,5s)-5-hydroxyadamantan-2-yl]-2-(oxetan-3-ylamino)pyrimidine-5-carboxamide;
4-cyclobutyl-2-[(1,1-dioxidotetrahydro-2H-thiopyran-4-yl)amino]-N-[(2r,5s)-5-hydroxyadamantan-2-yl]pyrimidine-5-carboxamide;
4-cyclopentyl-N-[(2r,5s)-5-hydroxyadamantan-2-yl]-2-oxetan-3-ylamino)pyrimidine-5-carboxamide;
4-cyclopentyl-2-[(1,1-dioxothian-4-yl)amino]-N-[(2r,5s)-5-hydroxyadamantan-2-yl]pyrimidine-5-carboxamide;
4-cyclopentyl-N-[(2r,5s)-5-hydroxyadamantan-2-yl]-2-[(2-hydroxy-2-methylpropyl)amino]pyrimidine-5-carboxamide;
4-cyclopentyl-N-[(2r,5s)-5-hydroxyadamantan-2-yl]-2-(2-hydroxyethylamino)pyrimidine-5-carboxamide;
4-cyclopentyl-N-[(2r,5s)-5-hydroxyadamantan-2-yl]-2-[(1-hydroxy-2-methylpropan-2-yl)amino]pyrimidine-5-carboxamide;
4-cyclopentyl-N-[(2r,5s)-5-hydroxyadamantan-2-yl]-2-(oxan-4-ylamino)pyrimidine-5-carboxamide;
4-cyclopentyl-N-[(2r,5s)-5-hydroxyadamantan-2-yl]-2-[[(3R)-oxolan-3-yl]amino]pyrimidine-5-carboxamide;
4-cyclopentyl-N-[(2r,5s)-5-hydroxyadamantan-2-yl]-2-[[(3S)-oxolan-3-yl]amino]pyrimidine-5-carboxamide;
4-ethoxy-N-[(2r,5s)-5-hydroxytricyclo[3.3.1.1^{3,7}]dec-2-yl]-2-(oxetan-3-ylamino)pyrimidine-5-carboxamide; or
N-[(2r,5s)-5-hydroxytricyclo[3.3.1.1^{3,7}]dec-2-yl]-4-(1-methylethoxy)-2-(oxetan-3-ylamino)pyrimidine-5-carboxamide.

In a 3^{rd} embodiment of formula (I), formula (I) is defined as being one or more compound(s) selected from the group consisting of:
4-cyclobutyl-N-(5-hydroxy-2-adamantyl)-2-[[(3S)-tetrahydrofuran-3-yl]amino]pyrimidine-5-carboxamide,
4-cyclobutyl-N-(5-hydroxy-2-adamantyl)-2-[[(3R)-tetrahydrofuran-3-yl]amino]pyrimidine-5-carboxamide,
2-[(1-acetyl-4-piperidyl)amino]-4-cyclopentyl-N-(5-hydroxy-2-adamantyl)pyrimidine-5-carboxamide,
4-cyclopentyl-N-(5-hydroxy-2-adamantyl)-2-[[(3R)-6-oxo-3-piperidyl]amino]pyrimidine-5-carboxamide,
4-cyclopentyl-2-[(1,1-dioxothiolan-3-yl)amino]-N-(5-hydroxy-2-adamantyl)pyrimidine-5-carboxamide,
4-cyclopentyl-N-(5-hydroxy-2-adamantyl)-2-[[(1S,2R)-2-hydroxycyclopentyl]amino]pyrimidine-5-carboxamide,
4-cyclopentyl-N-(5-hydroxy-2-adamantyl)-2-[[(2S)-tetrahydrofuran-2-yl]methylamino]pyrimidine-5-carboxamide,
4-cyclopentyl-N-(5-hydroxy-2-adamantyl)-2-[[(2R)-2-hydroxypropyl]amino]pyrimidine-5-carboxamide,
4-cyclopentyl-2-[[2-(dimethylamino)-2-oxo-ethyl]amino]-N-(5-hydroxy-2-adamantyl)pyrimidine-5-carboxamide,
4-cyclopentyl-N-(5-hydroxy-2-adamantyl)-2-(2-methoxyethylamino)pyrimidine-5-carboxamide,
4-cyclopentyl-N-(5-hydroxy-2-adamantyl)-2-[[(2R)-tetrahydrofuran-2-yl]methylamino]pyrimidine-5-carboxamide,
4-cyclopentyl-N-(5-hydroxy-2-adamantyl)-2-[[(2S)-2-hydroxypropyl]amino]pyrimidine-5-carboxamide,
4-cyclopentyl-N-(5-hydroxy-2-adamantyl)-2-[[(3S)-2-oxopyrrolidin-3-yl]amino]pyrimidine-5-carboxamide,
4-cyclopentyl-N-(5-hydroxy-2-adamantyl)-2-[[(3R)-5-oxopyrrolidin-3-yl]methylamino]pyrimidine-5-carboxamide,
2-[[(3R)-1-acetylpyrrolidin-3-yl]amino]-4-cyclopentyl-N-(5-hydroxy-2-adamantyl)pyrimidine-5-carboxamide,
4-cyclopentyl-N-(5-hydroxy-2-adamantyl)-2-[(1-methyl-5-oxo-pyrrolidin-3-yl)amino]pyrimidine-5-carboxamide,
4-cyclopentyl-2-[(1-ethyl-5-oxo-pyrrolidin-3-yl)amino]-N-(5-hydroxy-2-adamantyl)pyrimidine-5-carboxamide,
4-cyclopentyl-N-(5-hydroxy-2-adamantyl)-2-[[(2R)-1-methyl-5-oxo-pyrrolidin-2-yl]methylamino]pyrimidine-5-carboxamide,
4-cyclopentyl-N-(5-hydroxy-2-adamantyl)-2-[[(3S)-5-oxopyrrolidin-3-yl]methylamino]pyrimidine-5-carboxamide,
N-(5-hydroxy-2-adamantyl)-4-isopropyl-2-[[(3S)-tetrahydrofuran-3-yl]amino]pyrimidine-5-carboxamide,
N-(5-hydroxy-2-adamantyl)-4-isopropyl-2-[[(3R)-tetrahydrofuran-3-yl]amino]pyrimidine-5-carboxamide,
N-(5-hydroxy-2-adamantyl)-2-[[(3R)-oxolan-3-yl]amino]-4-(trifluoromethyl)pyrimidine-5-carboxamide,
N-(5-hydroxy-2-adamantyl)-2-[[(3S)-oxolan-3-yl]amino]-4-(trifluoromethyl)pyrimidine-5-carboxamide,
4-ethoxy-N-(5-hydroxy-2-adamantyl)-2-[[(3R)-tetrahydrofuran-3-yl]amino]pyrimidine-5-carboxamide,
4-ethoxy-N-(5-hydroxy-2-adamantyl)-2-[[(3S)-tetrahydrofuran-3-yl]amino]pyrimidine-5-carboxamide
4-(cyclobutoxy)-N-(5-hydroxy-2-adamantyl)-2-[[(3S)-tetrahydrofuran-3-yl]amino]pyrimidine-5-carboxamide
4-(cyclobutoxy)-N-(5-hydroxy-2-adamantyl)-2-[[(3R)-tetrahydrofuran-3-yl]amino]pyrimidine-5-carboxamide,
4-(cyclobutoxy)-N-(5-hydroxy-2-adamantyl)-2-(2-hydroxyethylamino)pyrimidine-5-carboxamide,
4-(cyclobutoxy)-N-(5-hydroxy-2-adamantyl)-2-(oxetan-3-ylamino)pyrimidine-5-carboxamide,
4-(cyclobutoxy)-N-(5-hydroxy-2-adamantyl)-2-[(2-hydroxy-1,1-dimethyl-ethyl)amino]pyrimidine-5-carboxamide,
4-(cyclobutoxy)-2-[(1,1-dioxothian-4-yl)amino]-N-(5-hydroxy-2-adamantyl)pyrimidine-5-carboxamide,
4-(cyclobutoxy)-N-(5-hydroxy-2-adamantyl)-2-(tetrahydropyran-4-ylamino)pyrimidine-5-carboxamide,
N-(5-hydroxy-2-adamantyl)-4-propoxy-2-[[(3R)-tetrahydrofuran-3-yl]amino]pyrimidine-5-carboxamide,
N-(5-hydroxy-2-adamantyl)-4-propoxy-2-[[(3S)-tetrahydrofuran-3-yl]amino]pyrimidine-5-carboxamide,
N-(5-hydroxy-2-adamantyl)-4-isopropoxy-2-[[(3S)-tetrahydrofuran-3-yl]amino]pyrimidine-5-carboxamide,
N-(5-hydroxy-2-adamantyl)-4-isopropoxy-2-[[(3R)-tetrahydrofuran-3-yl]amino]pyrimidine-5-carboxamide,
N-(5-hydroxy-2-adamantyl)-4-pyrrolidin-1-yl-2-[[(3R)-tetrahydrofuran-3-yl]amino]pyrimidine-5-carboxamide,
N-(5-hydroxy-2-adamantyl)-4-pyrrolidin-1-yl-2-[[(3S)-tetrahydro furan-3-yl]amino]pyrimidine-5-carboxamide,
N-(5-hydroxy-2-adamantyl)-4-pyrazol-1-yl-2-[[(3R)-tetrahydrofuran-3-yl]amino]pyrimidine-5-carboxamide,
N-(5-hydroxy-2-adamantyl)-4-pyrazol-1-yl-2-[[(3S)-tetrahydrofuran-3-yl]amino]pyrimidine-5-carboxamide,
4-Cyclopentyl-N-(5-hydroxy-2-adamantyl)-N-methyl-2-[[(3R)-oxolan-3-yl]amino]pyrimidine-5-carboxamide,
4-cyclopentyl-N-(5-hydroxy-2-adamantyl)-N-methyl-2-[[(3S)-oxolan-3-yl]amino]pyrimidine-5-carboxamide,
N-(5-carbamoyl-2-adamantyl)-4-cyclopentyl-2-[[(3S)-tetrahydrofuran-3-yl]amino]pyrimidine-5-carboxamide,
4-[[4-cyclopentyl-2-[[(3S)-tetrahydrofuran-3-yl]amino]pyrimidine-5-carbonyl]amino]adamantane-1-carboxylic acid, and
4-cyclopentyl-N-(5-methylsulfonyl-2-adamantyl)-2-[[(3S)-tetrahydrofuran-3-yl]amino]pyrimidine-5-carboxamide.

In a 4^{th} embodiment of formula (I), formula (I) is defined as being one or more compound(s) selected from the group consisting of:
4-cyclobutyl-N-(5-hydroxy-2-adamantyl)-2-[[(3S)-tetrahydrofuran-3-yl]amino]pyrimidine-5-carboxamide,
4-cyclobutyl-N-(5-hydroxy-2-adamantyl)-2-[[(3R)-tetrahydrofuran-3-yl]amino]pyrimidine-5-carboxamide,
2-[(1-acetyl-4-piperidyl)amino]-4-cyclopentyl-N-(5-hydroxy-2-adamantyl)pyrimidine-5-carboxamide,
4-cyclopentyl-N-(5-hydroxy-2-adamantyl)-2-[[(3R)-6-oxo-3-piperidyl]amino]pyrimidine-5-carboxamide,
4-cyclopentyl-2-[(1,1-dioxothiolan-3-yl)amino]-N-(5-hydroxy-2-adamantyl)pyrimidine-5-carboxamide,
4-cyclopentyl-N-(5-hydroxy-2-adamantyl)-2-[[(1S,2R)-2-hydroxycyclopentyl]amino]pyrimidine-5-carboxamide,
4-cyclopentyl-N-(5-hydroxy-2-adamantyl)-2-[[(2S)-tetrahydrofuran-2-yl]methylamino]pyrimidine-5-carboxamide,
4-cyclopentyl-N-(5-hydroxy-2-adamantyl)-2-[[(2R)-2-hydroxypropyl]amino]pyrimidine-5-carboxamide,
4-cyclopentyl-2-[[2-(dimethylamino)-2-oxo-ethyl]amino]-N-(5-hydroxy-2-adamantyl)pyrimidine-5-carboxamide,
4-cyclopentyl-N-(5-hydroxy-2-adamantyl)-2-(2-methoxyethylamino)pyrimidine-5-carboxamide,
4-cyclopentyl-N-(5-hydroxy-2-adamantyl)-2-[[(2R)-tetrahydrofuran-2-yl]methylamino]pyrimidine-5-carboxamide,
4-cyclopentyl-N-(5-hydroxy-2-adamantyl)-2-[[(2S)-2-hydroxypropyl]amino]pyrimidine-5-carboxamide,
4-cyclopentyl-N-(5-hydroxy-2-adamantyl)-2-[[(3S)-2-oxopyrrolidin-3-yl]amino]pyrimidine-5-carboxamide,
4-cyclopentyl-N-(5-hydroxy-2-adamantyl)-2-[[(3R)-5-oxopyrrolidin-3-yl]methylamino]pyrimidine-5-carboxamide,
2-[[(3R)-1-acetylpyrrolidin-3-yl]amino]-4-cyclopentyl-N-(5-hydroxy-2-adamantyl)pyrimidine-5-carboxamide,
4-cyclopentyl-N-(5-hydroxy-2-adamantyl)-2-[(1-methyl-5-oxo-pyrrolidin-3-yl)amino]pyrimidine-5-carboxamide,
4-cyclopentyl-2-[(1-ethyl-5-oxo-pyrrolidin-3-yl)amino]-N-(5-hydroxy-2-adamantyl)pyrimidine-5-carboxamide,
4-cyclopentyl-N-(5-hydroxy-2-adamantyl)-2-[[(2R)-1-methyl-5-oxo-pyrrolidin-2-yl]methylamino]pyrimidine-5-carboxamide,
4-cyclopentyl-N-(5-hydroxy-2-adamantyl)-2-[[(3S)-5-oxopyrrolidin-3-yl]methylamino]pyrimidine-5-carboxamide,
N-(5-hydroxy-2-adamantyl)-4-isopropyl-2-[[(3S)-tetrahydrofuran-3-yl]amino]pyrimidine-5-carboxamide,
N-(5-hydroxy-2-adamantyl)-4-isopropyl-2-[[(3R)-tetrahydrofuran-3-yl]amino]pyrimidine-5-carboxamide,
N-(5-hydroxy-2-adamantyl)-2-[[(3R)-oxolan-3-yl]amino]-4-(trifluoromethyl)pyrimidine-5-carboxamide,
N-(5-hydroxy-2-adamantyl)-2-[[(3S)-oxolan-3-yl]amino]-4-(trifluoromethyl)pyrimidine-5-carboxamide,
4-ethoxy-N-(5-hydroxy-2-adamantyl)-2-[[(3R)-tetrahydrofuran-3-yl]amino]pyrimidine-5-carboxamide,
4-ethoxy-N-(5-hydroxy-2-adamantyl)-2-[[(3S)-tetrahydrofuran-3-yl]amino]pyrimidine-5-carboxamide
4-(cyclobutoxy)-N-(5-hydroxy-2-adamantyl)-2-[[(3S)-tetrahydrofuran-3-yl]amino]pyrimidine-5-carboxamide
4-(cyclobutoxy)-N-(5-hydroxy-2-adamantyl)-2-[[(3R)-tetrahydrofuran-3-yl]amino]pyrimidine-5-carboxamide,
4-(cyclobutoxy)-N-(5-hydroxy-2-adamantyl)-2-(2-hydroxyethylamino)pyrimidine-5-carboxamide,
4-(cyclobutoxy)-N-(5-hydroxy-2-adamantyl)-2-(oxetan-3-ylamino)pyrimidine-5-carboxamide,
4-(cyclobutoxy)-N-(5-hydroxy-2-adamantyl)-2-[(2-hydroxy-1,1-dimethyl-ethyl)amino]pyrimidine-5-carboxamide,
4-(cyclobutoxy)-2-[(1,1-dioxothian-4-yl)amino]-N-(5-hydroxy-2-adamantyl)pyrimidine-5-carboxamide,
4-(cyclobutoxy)-N-(5-hydroxy-2-adamantyl)-2-(tetrahydropyran-4-ylamino)pyrimidine-5-carboxamide,
N-(5-hydroxy-2-adamantyl)-4-propoxy-2-[[(3R)-tetrahydrofuran-3-yl]amino]pyrimidine-5-carboxamide,
N-(5-hydroxy-2-adamantyl)-4-propoxy-2-[[(3S)-tetrahydrofuran-3-yl]amino]pyrimidine-5-carboxamide,
N-(5-hydroxy-2-adamantyl)-4-isopropoxy-2-[[(3S)-tetrahydrofuran-3-yl]amino]pyrimidine-5-carboxamide,
N-(5-hydroxy-2-adamantyl)-4-isopropoxy-2-[[(3R)-tetrahydrofuran-3-yl]amino]pyrimidine-5-carboxamide,
4-Cyclopentyl-N-(5-hydroxy-2-adamantyl)-N-methyl-2-[[(3R)-oxolan-3-yl]amino]pyrimidine-5-carboxamide,
4-cyclopentyl-N-(5-hydroxy-2-adamantyl)-N-methyl-2-[[(3S)-oxolan-3-yl]amino]pyrimidine-5-carboxamide and
N-(5-carbamoyl-2-adamantyl)-4-cyclopentyl-2-[[(3S)-tetrahydrofuran-3-yl]amino]pyrimidine-5-carboxamide.

In a 5^{th} embodiment of formula (I), formula (I) is defined as being one or more compound(s) selected from the group consisting of:
4-cyclobutyl-N-(5-hydroxy-2-adamantyl)-2-[[(3R)-tetrahydrofuran-3-yl]amino]pyrimidine-5-carboxamide,
2-[(1-acetyl-4-piperidyl)amino]-4-cyclopentyl-N-(5-hydroxy-2-adamantyl)pyrimidine-5-carboxamide,
4-cyclopentyl-N-(5-hydroxy-2-adamantyl)-2-[[(3R)-6-oxo-3-piperidyl]amino]pyrimidine-5-carboxamide,
4-cyclopentyl-2-[(1,1-dioxothiolan-3-yl)amino]-N-(5-hydroxy-2-adamantyl)pyrimidine-5-carboxamide,
4-cyclopentyl-N-(5-hydroxy-2-adamantyl)-2-[[(1S,2R)-2-hydroxycyclopentyl]amino]pyrimidine-5-carboxamide,
4-cyclopentyl-N-(5-hydroxy-2-adamantyl)-2-[[(2S)-tetrahydrofuran-2-yl]methylamino]pyrimidine-5-carboxamide,
4-cyclopentyl-N-(5-hydroxy-2-adamantyl)-2-[[(2R)-2-hydroxypropyl]amino]pyrimidine-5-carboxamide,
4-cyclopentyl-N-(5-hydroxy-2-adamantyl)-2-(2-methoxyethylamino)pyrimidine-5-carboxamide,
4-cyclopentyl-N-(5-hydroxy-2-adamantyl)-2-[[(2R)-tetrahydrofuran-2-yl]methylamino]pyrimidine-5-carboxamide,
4-cyclopentyl-N-(5-hydroxy-2-adamantyl)-2-[[(2S)-2-hydroxypropyl]amino]pyrimidine-5-carboxamide,
2-[[(3R)-1-acetylpyrrolidin-3-yl]amino]-4-cyclopentyl-N-(5-hydroxy-2-adamantyl)pyrimidine-5-carboxamide,
4-cyclopentyl-N-(5-hydroxy-2-adamantyl)-2-[(1-methyl-5-oxo-pyrrolidin-3-yl)amino]pyrimidine-5-carboxamide,
4-cyclopentyl-2-[(1-ethyl-5-oxo-pyrrolidin-3-yl)amino]-N-(5-hydroxy-2-adamantyl)pyrimidine-5-carboxamide,
4-cyclopentyl-N-(5-hydroxy-2-adamantyl)-2-[[(2R)-1-methyl-5-oxo-pyrrolidin-2-yl]methylamino]pyrimidine-5-carboxamide,
4-cyclopentyl-N-(5-hydroxy-2-adamantyl)-2-[[(3S)-5-oxopyrrolidin-3-yl]methylamino]pyrimidine-5-carboxamide,
N-(5-hydroxy-2-adamantyl)-2-[[(3R)-oxolan-3-yl]amino]-4-(trifluoromethyl)pyrimidine-5-carboxamide,
4-(cyclobutoxy)-N-(5-hydroxy-2-adamantyl)-2-[[(3S)-tetrahydrofuran-3-yl]amino]pyrimidine-5-carboxamide,
4-(cyclobutoxy)-N-(5-hydroxy-2-adamantyl)-2-[[(3R)-tetrahydrofuran-3-yl]amino]pyrimidine-5-carboxamide,
4-(cyclobutoxy)-N-(5-hydroxy-2-adamantyl)-2-(2-hydroxyethylamino)pyrimidine-5-carboxamide,
4-(cyclobutoxy)-N-(5-hydroxy-2-adamantyl)-2-(oxetan-3-ylamino)pyrimidine-5-carboxamide,
N-(5-hydroxy-2-adamantyl)-4-propoxy-2-[[(3R)-tetrahydrofuran-3-yl]amino]pyrimidine-5-carboxamide,
N-(5-hydroxy-2-adamantyl)-4-propoxy-2-[[(3S)-tetrahydrofuran-3-yl]amino]pyrimidine-5-carboxamide,
N-(5-hydroxy-2-adamantyl)-4-isopropoxy-2-[[(3S)-tetrahydrofuran-3-yl]amino]pyrimidine-5-carboxamide,
N-(5-hydroxy-2-adamantyl)-4-isopropoxy-2-[[(3R)-tetrahydrofuran-3-yl]amino]pyrimidine-5-carboxamide and
N-(5-carbamoyl-2-adamantyl)-4-cyclopentyl-2-[[(3S)-tetrahydrofuran-3-yl]amino]pyrimidine-5-carboxamide.

### Particular Classes of Compounds

Particular classes of compounds of the present invention are disclosed in Table A using combinations of the definitions described hereinabove. For example, ‘a’ in the column headed R₁ in the table refers to definition (a) given for R₁ hereinabove and ‘1’ refers to the first definition given for the variables in the compound of formula (1) in the first embodiment of formula (I) in the beginning of the description. A “-”, in the table cell means the original definition used in the given numbered embodiment for that class (row) is valid.

TABLE A

| Class | Embodiment | R¹ | R² | R³ | R⁴ |
|---|---|---|---|---|---|
| 1 | 1^{st} | a | a | — | — |
| 2 | 1^{st} | a | — | a | — |
| 3 | 1^{st} | a | — | — | a |
| 4 | 1^{st} | a | b | — | — |
| 5 | 1^{st} | a | — | b | — |
| 6 | 1^{st} | a | — | — | b |
| 7 | 1^{st} | a | a | — | — |
| 8 | 1^{st} | a | — | b | a |
| 9 | 1^{st} | a | — | b | b |
| 10 | 1^{st} | a | b | d | a |
| 11 | 1^{st} | a | — | d | b |
| 12 | 1^{st} | a | — | — | b |
| 13 | 1^{st} | a | b | d | b |
| 14 | 1^{st} | f | a | — | — |
| 15 | 1^{st} | f | — | a | — |
| 16 | 1^{st} | f | — | — | a |
| 17 | 1^{st} | f | b | — | — |
| 18 | 1^{st} | f | — | b | — |
| 19 | 1^{st} | f | — | — | b |
| 20 | 1^{st} | f | a | — | — |
| 21 | 1^{st} | f | — | b | a |
| 22 | 1^{st} | f | — | b | b |
| 23 | 1^{st} | f | b | d | a |
| 24 | 1^{st} | f | — | d | b |
| 25 | 1^{st} | f | — | — | b |
| 26 | 1^{st} | f | b | d | b |
| 27 | 2^{nd} | — | a | — | — |
| 28 | 2^{nd} | — | a | — | a |
| 29 | 2^{nd} | — | a | — | b |
| 30 | 2^{nd} | b | a | — | a |
| 31 | 2^{nd} | b | a | — | b |
| 32 | 2^{nd} | — | b | — | a |
| 33 | 2^{nd} | — | b | — | b |
| 34 | 2^{nd} | b | b | — | a |
| 35 | 2^{nd} | b | b | — | b |
| 36 | 2^{nd} | f | b | — | a |
| 37 | 2^{nd} | f | b | — | b |

In a further particular embodiment, Formula (I) is selected as one compound from the list provided to the 3^{rd} embodiment, above.

In another further particular embodiment, Formula (I) is selected as a pharmaceutically acceptable salt of one compound from the list provided to the 3^{rd} embodiment, above.

In an even further particular embodiment, Formula (I) is selected as one compound from the list provided to the 4^{th} embodiment, above.

In another even further particular embodiment, Formula (I) is selected as a pharmaceutically acceptable salt of one compound from the list provided to the 4^{th} embodiment, above.

### Process

Another aspect of the present invention provides processes for preparing a compound of formula (I) or a pharmaceutically acceptable salt thereof which process, wherein variable groups are, unless otherwise specified, as defined for formula (I) and comprises any one of the following processes;

a) Suitable for when R1 is a Carbon Based Substituent

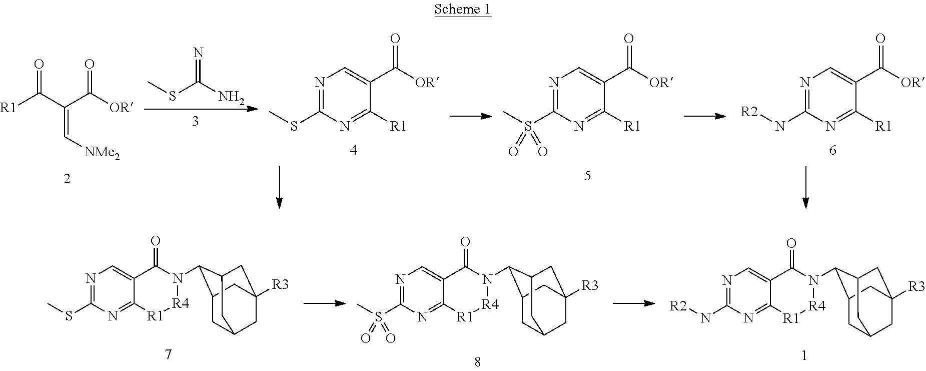

According to this method, a compound of formula 2 is converted to a compound of formula 4 by treatment with methylsulfonylformadine 3. The compound of formula 4 is then oxidised to give a sulfone of formula 5, which is reacted with an appropriate amine to give the aminopyrimidine 6. 6 is then converted to the desired compound 1 by cleavage of the ester to the acid and formation of the amide with the appropriate amine. Alternatively 4 can be converted to the desired compound 1 by reversing the amine addition and amide formation steps. Thus 4 can be converted to 7 by cleavage of the ester to the acid and amide formation with an appropriate amine, then 7 can be oxidised to the sulfone 8. 8 can then be converted to the desired compound 1 by addition of an appropriate amine.

Methods for conversion of compounds of formula 2 to pyrimidines of formula 4 are well known in the art and examples are described in the following patent reference; WO2006050476. The compound of formula 2 is treated with isothiourea sulphate 3 in an inert solvent (e.g. DMF) with an appropriate base (e.g. sodium acetate) and heated at temperatures of between 50-100° C., ideally at 80-90° C. to give pyrimidines of formula 4. Methods for conversion of thioethers of formula 4 to sulfones of formula 5 (or 7 to 8) are well known in the art and examples are described in the following patent reference; WO2006050476. The compound of formula 4 (or 7) is treated with an appropriate oxidising agent (e.g. 3-chlorobenzoperoxoic acid or oxone) in an inert solvent (e.g. dichloromethane), typically at ambient temperature. Methods for conversion of compounds of formula 5 to compounds of formula 6 (or 8 to 1) are well known in the art and examples are described in the following references; WO2006050476, Synth. Commun., 2007, 37, 2231; Bioorg. Med. Chem., 2005, 13, 5717. The compound of formula 5 (or 8) is treated with an appropriate nucleophilic reagent in an inert solvent (e.g. butyronitrile, THF, DMF, 1,4-dioxane) at temperatures ranging from ambient temperature to 150° C. Methods for conversion of compounds of formula 6 to pyrimidines of formula 1 (or 4 to 7) are well known to the art. Cleavage of a compound of formula 6 (or 4) to the corresponding carboxylic acid will be dependent on the nature of the ester group used and many procedures are outlined in the following reference; T. W. Green, Protective Groups in Organic Synthesis, John Wiley and Sons, 1991. For example, in the case where R′ represents lower alkyl (e.g. methyl or ethyl), the reaction can be carried out by hydrolysis with a suitable base such as an alkaline metal hydroxide (e.g. sodium hydroxide, potassium hydroxide or lithium hydroxide) in a suitable solvent (e.g. methanol, THF, water) at temperatures ranging from 0-50° C. but preferably at ambient temperature. Formation of an amide from a carboxylic acid is a process well known to the art. Typical processes include, but are not limited to, formation of an acyl halide by treatment with a suitable reagent (e.g. oxalyl chloride, POCl₃) in a suitable solvent such as dichloromethane or N,N-dimethylformamide for example at temperatures ranging from 0-50° C. but preferably at ambient temperature. Alternatively, in situ conversion of the acid to an active ester derivative may be utilised with the addition of a suitable coupling agent (or combination of agents) to form an active ester such as HATU, HOBT, and EDAC for example, optionally in the presence of a suitable base such as triethylamine or DIPEA. Typically the reaction is carried out at temperatures ranging from 0-50° C. but preferably at ambient temperature.

b) Suitable for when R1 is a Non-Carbon Based Substituent

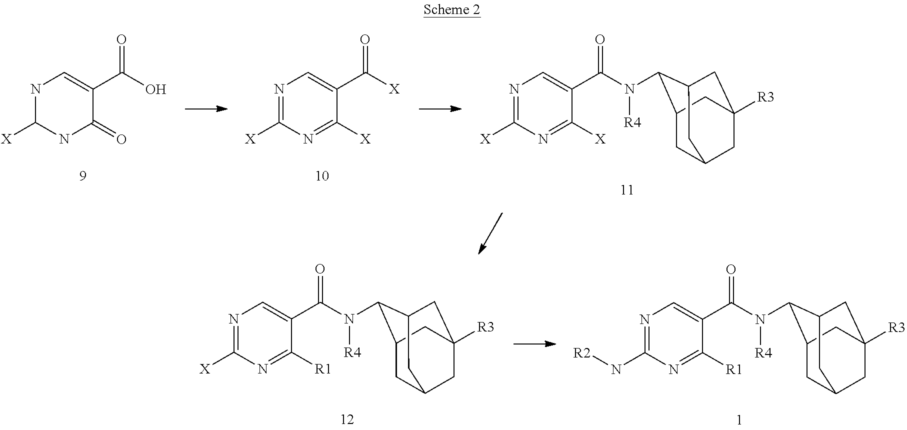

According to this method, a pyrimidinedione acid of formula 9 is halogenated to give a di-halo acyl halide (or equivalent) compound of formula 10. Compound 10 is then treated with an appropriate amine to give compounds of formula 11. The di-halo amide is then treated with a stoichiometric quantity of an appropriate nucleophile (R1) to give a compound of formula 12 and then reacted with an appropriate amine to give the desired compound of formula 1.

Methods for conversion of compounds of formula 9 to compounds of formula 10 are well known in the art and examples are described in the following references; J. Med. Chem., 2007, 50, 591. The compound of formula 9 is treated with a suitable halogenating system (e.g. POCl₃/PCl₅ or Cl₂P(═O)OPh) in an inert solvent (e.g. DMF) or neat and heated at temperatures of between 50-190° C., ideally at reflux to give halo pyrimidines. Methods for conversion of compounds of formula 10 to amides of formula 11 are well known in the art and examples are described in the following references; J. Org. Chem., 2007, 72, 7058; Bioorg. Med. Chem. Lett., 2007, 17, 1951. The compound of formula 10 is treated with an appropriate amine in the presence of a suitable base (e.g. DIPEA) in a suitable solvent (e.g. dichloromethane) at temperatures of 0-50° C., typically at 0° C. to ambient temperature. Methods for conversion of compounds of formula 11 to compounds of formula 12 are well known in the art and examples are described in the following references; J. Med. Chem., 2007, 50, 591. Compounds of formula 11 are treated with appropriate nucleophiles in an inert solvent (e.g. DMF, butyronitrile, dichloromethane) in the presence of an appropriate base (e.g. potassium carbonate, sodium carbonate, DIPEA) at temperatures ranging from ambient temperature to 100° C. dependant of the nucleophilicity of the reagent to give compounds of formula 12. Optionally, the anion of the nucleophile may be prepared by treatment with a suitable base (e.g. sodium hydride, lithium hexamethyldisilazide). It will be appreciated by those skilled in the art that regioisomeric mixtures may result in this reaction and that separation techniques may be required to obtain the desired regiosiomer. Methods for conversion of compounds of formula 12 to compounds of formula 1 are analogous to those previously outlined for the conversion of compounds of formula 11 to compounds of formula 12 described above. It will be appreciated by those skilled in the art that where R1=NR4R5 an analogous method can also be used to convert 11 to 1 directly.

A significant number of β-ketoesters are commercially available as listed in the Available Chemicals Directory and a further number have been described in the chemical literature. A listing of many of the methods suitable for preparation of β-ketoesters is contained within ‘Comprehensive Organic Transformations; A Guide to Functional Group Preparations’, VCH Publishers, Inc, NY, 1989, p 685, 694 & 768]. Additional methods may be found in ‘Advanced Organic Chemistry’, 3^{rd} Ed, J. Wiley & Sons, Inc, NY, 1985 p 437 & 823.

It will be appreciated that certain of the various substituents in the compounds of the present invention may be generated by conventional functional group modifications either prior to or immediately following the processes mentioned above, and as such are included in the process aspect of the invention. Such reactions and modifications include, reduction of substituents, oxidation of substituents and alkylation of substituents, for example, alkylation reactions such as conversion of a secondary amide to a primary amide typically carried out using strong base (e.g. sodium hydride or lithium or potassium hexamethyldisilylazides) and a suitable alkylating agent (e.g. methyl iodide). The reagents and reaction conditions for such procedures are well known in the chemical art. It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example hydroxylamine, or with hydrazine. A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon. A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon. The protecting groups may be removed at any convenient stage in the synthesis using conventional techniques well known in the chemical art.

As stated hereinbefore the compounds defined in the present invention possess 11βHSD1 inhibitory activity. These properties may be assessed using the following assay.

### Assays

The conversion of cortisone to the active steroid cortisol by 11βHSD1 oxo-reductase activity, can be measured using a competitive homogeneous time resolved fluorescence assay (HTRF) (CisBio International, R&D, Administration and Europe Office, In Vitro Technologies—HTRF®/Bioassays BP 84175, 30204 Bagnols/Cèze Cedex, France. Cortisol bulk HTRF kit: Cat No. 62CO2PEC).

The evaluation of compounds described herein was carried out using a baculovirus expressed N terminal 6-His tagged full length human 11βHSD1 enzyme (*1). The enzyme was purified from a detergent solublised cell lysate, using a copper chelate column. Inhibitors of 11βHSD1 reduce the conversion of cortisone to cortisol, which is identified by an increase in signal, in the above assay.

Compounds to be tested were dissolved in dimethyl sulphoxide (DMSO) to 10 mM and diluted further in assay buffer containing 1% DMSO to 10 fold the final assay concentration. Diluted compounds were then plated into black 384 well plates (Matrix, Hudson N.H., USA).

The assay was carried out in a total volume of 20 μl consisting of cortisone (Sigma, Poole, Dorset, UK, 160 nM), glucose-6-phosphate (Roche Diagnostics, 1 mM), NADPH (Sigma, Poole, Dorset, 100 μM), glucose-6-phosphate dehydrogenase (Roche Diagnostics, 12.5 μg/ml), EDTA (Sigma, Poole, Dorset, UK, 1 mM), assay buffer (K₂HPO₄/KH₂PO₄, 100 mM) pH 7.5, recombinant 11βHSD1 [using an appropriate dilution to give a viable assay window—an example of a suitable dilution may be 1 in 1000 dilution of stock enzyme] plus test compound. The assay plates were incubated for 25 minutes at 37° C. after which time the reaction was stopped by the addition of 10 μl of 0.5 mM glycerrhetinic acid plus conjugated cortisol (D2**). 10 μl of anti-cortisol Cryptate was then added and the plates sealed and incubated for 6 hours at room temperature. Fluorescence at 665 nm and 620 nm was measured and the 665 nm:620 nm ratio calculated using an Envision plate reader.

These data were then used to calculate IC₅₀ values for each compound (Origin 7.5, Microcal software, Northampton Mass., USA).

### *1 The Journal of Biological Chemistry, 1991, Vol. 266, No 25, pp 16653-16658.

**D2 is a cortisol tracer which competes with native cortisol produced in the reaction for the monoclonal anti-cortisol antibody labelled with europium-cryptate. The antibody will fluoresce when it is bound to the cortisol tracer complex (D2 labelled with cortisol and glycyrrhetinic acid coupled together). Thus providing an accurate measure of 11beta-HSD1 activity.

Most compounds of the present invention typically show an IC₅₀ of less than 3 μM, and preferably less than 1 μM.

For example, the following results were obtained:

| Ex. No. | IC₅₀ (μM) |
|---|---|
| 1 | 0.031 |
| 2 | 0.023 |
| 3 | 0.036 |
| 4 | 0.023 |
| 5 | 0.024 |
| 6 | 0.023 |
| 7 | 0.023 |
| 8 | 0.022 |
| 9 | 0.031 |
| 10 | 0.018 |
| 11 | 0.015 |
| 12 | 0.020 |
| 13 | 0.067 |
| 14 | 0.028 |
| 15 | 0.021 |
| 16 | 0.020 |
| 17 | 0.015 |
| 18 | 0.023 |
| 19 | 0.018 |
| 20 | 0.046 |
| 21 | 0.042 |
| 22 | 0.025 |
| 23 | 0.095 |
| 24 | 0.045 |
| 25 | 0.037 |
| 26 | 0.016 |
| 27 | 0.009 |
| 28 | 0.012 |
| 29 | 0.016 |
| 30 | 0.029 |
| 31 | 0.043 |
| 32 | 0.037 |
| 33 | 0.019 |
| 34 | 0.021 |
| 35 | 0.022 |
| 36 | 0.009 |
| 37 | 0.777 |
| 38 | 1.598 |
| 39 | 0.708 |
| 40 | 2.267 |
| 41 | 0.046 |
| 42 | 0.032 |
| 43 | 0.017 |
| 44 | 0.424 |
| 45 | 0.273 |

According to a further aspect of the invention there is provided a pharmaceutical composition, which comprises a compound of the Examples, or a pharmaceutically-acceptable salt thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing). In general, compositions in a form suitable for oral use are preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

We have found that the compounds defined in the present invention, or a pharmaceutically-acceptable salt thereof, are effective 11βHSD1 inhibitors, and accordingly have value in the treatment of disease states associated with metabolic syndrome.

It is to be understood that where the term “metabolic syndrome” is used herein, this relates to metabolic syndrome as defined in 1) and/or 2) on page 4 herein, or any other recognised definition of this syndrome. Synonyms for “metabolic syndrome” used in the art include Reaven's Syndrome, Insulin Resistance Syndrome and Syndrome X. It is to be understood that where the term “metabolic syndrome” is used herein it also refers to Reaven's Syndrome, Insulin Resistance Syndrome and Syndrome X.

According to a further aspect of the present invention there is provided a compound of formula (1), or a pharmaceutically-acceptable salt thereof, as defined hereinbefore for use in a method of prophylactic or therapeutic treatment of a warm-blooded animal, such as man.

Thus according to this aspect of the invention there is provided a compound of formula (I), or a pharmaceutically-acceptable salt thereof, as defined hereinbefore for use as a medicament.

According to another feature of the invention there is provided the use of a compound of formula (I), or a pharmaceutically-acceptable salt thereof, as defined hereinbefore in the manufacture of a medicament for use in the production of an 11βHSD1 inhibitory effect in a warm-blooded animal, such as man. In some embodiments, the animal will be in need of such treatment.

Where production of or producing an 11βHSD1 inhibitory effect is referred to suitably this refers to the treatment of metabolic syndrome. Alternatively, where production of an 11βHSD1 inhibitory effect is referred to this refers to the treatment of diabetes, obesity, hyperlipidaemia, hyperglycaemia, hyperinsulinemia or hypertension. In particular where production of an 11βHSD1 inhibitory effect is referred to this refers to the treatment of diabetes and obesity. In one aspect, type 2 diabetes. In another aspect, obesity. Alternatively, where production of an 11βHSD1 inhibitory effect is referred to this refers to the treatment of glaucoma, osteoporosis, tuberculosis, dementia, cognitive disorders or depression.

Alternatively, where production of an 11βHSD1 inhibitory effect is referred to this refers to the treatment of cognitive disorders, such as improving the cognitive ability of an individual, for example by improvement of verbal fluency, verbal memory or logical memory, or for treatment of mild cognitive disorders. See for example WO03/086410 and references contained therein, and Proceedings of National Academy of Sciences (PNAS), 2001, 98(8), 4717-4721.

Alternatively, where production of an 11βHSD1 inhibitory effect is referred to this refers to the treatment of, delaying the onset of and/or reducing the risk of atherosclerosis—see for example J. Experimental Medicine, 2005, 202(4), 517-527.

Alternatively, where production of an 11βHSD1 inhibitory effect is referred to this refers to the treatment of Alzheimers and/or neurodegenerative disorders.

According to a further feature of this aspect of the invention there is provided a method for producing an 11βHSD1 inhibitory effect in a warm-blooded animal, such as man, in need of such treatment which comprises administering to said animal an effective amount of a compound of formula (I), or a pharmaceutically-acceptable salt thereof.

In addition to their use in therapeutic medicine, the compounds of formula (I), or a pharmaceutically-salt thereof, are also useful as pharmacological tools in the development and standardisation of in vitro and in vivo test systems for the evaluation of the effects of inhibitors of 11βHSD1 in laboratory animals such as cats, dogs, rabbits, monkeys, rats and mice, as part of the search for new therapeutic agents.

The inhibition of 11βHSD1 described herein may be applied as a sole therapy or may involve, in addition to the subject of the present invention, one or more other substances and/or treatments. Such conjoint treatment may be achieved by way of the simultaneous, sequential or separate administration of the individual components of the treatment. Simultaneous treatment may be in a single tablet or in separate tablets. For example agents than might be co-administered with 11βHSD1 inhibitors, particularly those of the present invention, may include the following main categories of treatment:

1) Insulin and insulin analogues;

2) Insulin secretagogues including sulphonylureas (for example glibenclamide, glipizide), prandial glucose regulators (for example repaglinide, nateglinide), glucagon-like peptide 1 agonist (GLP1 agonist) (for example exenatide, liraglutide) and dipeptidyl peptidase IV inhibitors (DPP-IV inhibitors);

3) Insulin sensitising agents including PPARγ agonists (for example pioglitazone and rosiglitazone);

4) Agents that suppress hepatic glucose output (for example metformin);

5) Agents designed to reduce the absorption of glucose from the intestine (for example acarbose);

6) Agents designed to treat the complications of prolonged hyperglycaemia; e.g. aldose reductase inhibitors

7) Other anti-diabetic agents including phosotyrosine phosphatase inhibitors, glucose 6-phosphatase inhibitors, glucagon receptor antagonists, glucokinase activators, glycogen phosphorylase inhibitors, fructose 1,6 bisphosphastase inhibitors, glutamine:fructose-6-phosphate amidotransferase inhibitors

8) Anti-obesity agents (for example sibutramine and orlistat);

9) Anti-dyslipidaemia agents such as, HMG-CoA reductase inhibitors (statins, eg pravastatin); PPARα agonists (fibrates, eg gemfibrozil); bile acid sequestrants (cholestyramine); cholesterol absorption inhibitors (plant stanols, synthetic inhibitors); ileal bile acid absorption inhibitors (IBATi), cholesterol ester transfer protein inhibitors and nicotinic acid and analogues (niacin and slow release formulations);

10) Antihypertensive agents such as, β blockers (eg atenolol, inderal); ACE inhibitors (eg lisinopril); calcium antagonists (eg. nifedipine); angiotensin receptor antagonists (eg candesartan), a antagonists and diuretic agents (eg. furosemide, benzthiazide);

11) Haemostasis modulators such as, antithrombotics, activators of fibrinolysis and antiplatelet agents; thrombin antagonists; factor Xa inhibitors; factor VIIa inhibitors; antiplatelet agents (eg. aspirin, clopidogrel); anticoagulants (heparin and Low molecular weight analogues, hirudin) and warfarin;

12) Anti-inflammatory agents, such as non-steroidal anti-inflammatory drugs (eg. aspirin) and steroidal anti-inflammatory agents (eg. cortisone); and

13) Agents that prevent the reabsorption of glucose by the kidney (SGLT inhibitors).

In the above other pharmaceutical composition, process, method, use and medicament manufacture features, the alternative and preferred embodiments of the compounds of the invention described herein also apply.

### EXAMPLES

The invention will now be illustrated by the following Examples in which, unless stated otherwise:

(i) temperatures are given in degrees Celsius (° C.); operations were carried out at room or ambient temperature (RT), that is, at a temperature in the range of 18-25° C. and under an atmosphere of an inert gas such as argon;

(ii) evaporation of solvent was carried out using a rotary evaporator under reduced pressure (600-4000 Pa; 4.5-30 mmHg) with a bath temperature of up to 60° C.;

(iii) chromatography means flash chromatography on silica gel (FCC);

(iv) in general, the course of reactions was followed by TLC and reaction times are given for illustration only;

(v) yields are given for illustration only and are not necessarily those which can be obtained by diligent process development; preparations were repeated if more material was required;

(vi) where given, NMR data (¹H) is in the form of delta values for major diagnostic protons, given in parts per million (ppm) relative to tetramethylsilane (TMS), determined at 300 or 400 MHz (unless otherwise stated) using perdeuterio dimethyl sulfoxide (DMSO-d₆) as solvent, unless otherwise stated; peak multiplicities are shown thus: s, singlet; d, doublet; dd, doublet of doublets; dt, doublet of triplets; dm, doublet of multiplets; t, triplet, q, quartet, qn, quintet, m, multiplet; br, broad; protons attached to oxygen or nitrogen may give rise to very broad peaks which are not reported;

(vii) chemical symbols have their usual meanings; SI units and symbols are used;

(viii) solvent ratios are given in volume:volume (v/v) terms;

(ix) mass spectra (MS) were run with an electron energy of 70 electron volts in the chemical ionisation (CI) mode using a direct exposure probe; where indicated ionisation was effected by electron impact (EI), fast atom bombardment (FAB) or electrospray (ESP); values for m/z are given; generally, only ions which indicate the parent mass are reported;

(x) The following abbreviations may be used below or in the process section hereinbefore:
Me methyl
Et ethyl
DMF N, N-dimethylformamide
DCM dichloromethane
DME 1,2-dimethoxyethane
TFA trifluoroacetic acid
THF tetrahydrofuran
HATU 2-(3H-[1,2,3]Triazolo[4,5-b]pyridin-3-yl)-1,1,3,3-tetramethylisouronium hexafluorophosphate(V)
DMSO dimethylsulfoxide
DIPEA diisopropylethylamine
Ac acetyl
BINAP 2,2′-bis(diphenylphosphino)-1,1′binaphthyl
FCC flash column chromatography
sat. Saturated
aq. Aqueous
h hour

### Example 1

### 4-cyclobutyl-N-(5-hydroxy-2-adamantyl)-2-[[(3S)-tetrahydrofuran-3-yl]amino]pyrimidine-5-carboxamide

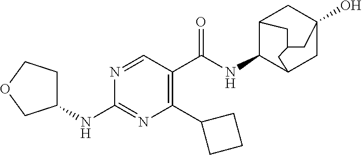

A solution of DIPEA (0.265 mL, 1.52 mmol) was added to a stirred suspension of (1S,4R)-4-aminoadamantan-1-ol.hydrochloride (85 mg, 0.42 mmol), (S)-4-cyclobutyl-2-(tetrahydrofuran-3-ylamino)pyrimidine-5-carboxylic acid (Intermediate 1, 100 mg, 0.38 mmol) and HATU (173 mg, 0.46 mmol) in DMF (1.5 mL) at RT under nitrogen. The resulting orange solution was stirred at RT for 3 h. The reaction mixture was concentrated and diluted with EtOAc, and washed sequentially with water and sat. brine. The organic layer was dried over MgSO₄, filtered and evaporated to afford crude product. The crude product was purified by FCC, elution gradient 0 to 10% MeOH in EtOAc, to afford the title compound (138 mg, 88%) as a white solid; ¹H NMR (400 MHz) 1.31 (2H, d), 1.61 (4H, d), 1.67-1.80 (3H, m), 1.95 (7H, m), 2.12 (3H, m), 2.28 (2H, m), 3.54 (1H, m), 3.72 (1H, m), 3.87 (4H, m), 4.41 (2H, s), 7.56 (1H, d), 7.96 (1H, d), 8.15 (1H, s); m/z MH⁺=413; HPLC tR=1.56 min.

### Intermediate 1

### (S)-4-Cyclobutyl-2-(tetrahydrofuran-3-ylamino)pyrimidine-5-carboxylic acid

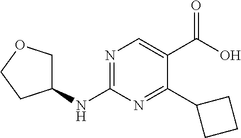

2 M aq. NaOH (1.478 mL, 2.96 mmol) was added to (S)-methyl 4-cyclobutyl-2-(tetrahydrofuran-3-ylamino)pyrimidine-5-carboxylate (Intermediate 2, 410 mg, 1.48 mmol) in MeOH (10 mL). The resulting mixture was stirred at RT for 16 h. The reaction mixture was concentrated, diluted with water and washed with diethyl ether. The aqueous solution was acidified with 1 M aq. citric acid. The resulting precipitate was collected by filtration, washed with water and dried under vacuum at 50° C. to afford the title compound (250 mg, 64%) as a white solid; ¹H NMR (400 MHz) 1.75-1.90 (1H, m), 1.92-2.10 (2H, m), 2.24-2.45 (5H, m), 3.58-3.68 (1H, m), 3.72-3.80 (1H, m), 3.85-4.05 (2H, m), 4.30-4.42 (1H, m), 4.45-4.60 (1H, m), 7.97-8.15 (1H, m), 8.63-8.72 (1H, m), 8.85 (1H, s), 12.64 (1H, s); m/z MH⁺=264; HPLC tR=1.49 min.

### Intermediate 2

### (S)-Methyl 4-cyclobutyl-2-(tetrahydrofuran-3-ylamino)pyrimidine-5-carboxylate

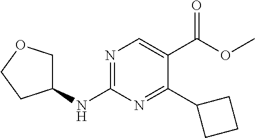

(S)-Tetrahydrofuran-3-amine hydrochloride (274 mg, 2.22 mmol) was added to methyl 4-cyclobutyl-2-(methylsulfonyl)pyrimidine-5-carboxylate (Intermediate 3, 400 mg, 1.48 mmol) and DIPEA (0.516 mL, 2.96 mmol) in THF (10 mL). The resulting solution was stirred at RT for 16 h. The reaction mixture was evaporated to dryness, taken up in DCM and washed sequentially with 1 M citric acid, sat. NaHCO₃ and sat. brine. The organic layer was dried over MgSO₄, filtered and evaporated to afford crude product as an oil (410 mg) that was used without further purification; ¹H NMR (400 MHz) 1.75-1.77 (1H, m), 1.80-1.87 (1H, m), 2.05-2.19 (3H, m), 2.20-2.38 (3H, d), 3.50-3.58 (1H, m), 3.62-3.72 (1H, m), 3.70 (3H, s), 3.77-3.84 (1H, m), 4.09-4.18 (1H, m), 4.21-4.30 (1H, m), 4.40-4.50 (1H, m), 8.00-8.12 (1H, m), 8.55-8.62 (1H, m); m/z MH⁺=278; HPLC tR=2.01 min.

### Intermediate 3

### Methyl 4-cyclobutyl-2-(methylsulfonyl)pyrimidine-5-carboxylate

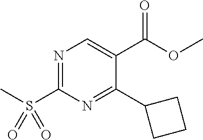

Oxone (31 g, 50.4 mmol) was added portionwise to methyl 4-cyclobutyl-2-(methylthio)pyrimidine-5-carboxylate (Intermediate 4, 6 g, 25.2 mmol) in acetonitrile (50 mL) and water (50 mL). The resulting suspension was stirred at RT for 16 h. The reaction mixture was evaporated. Sat. aq. NaHCO₃ (200 mL) was added and the mixture was extracted with DCM. The organic phases were combined and dried over MgSO₄, filtered and evaporated to afford methyl 4-cyclobutyl-2-(methylsulfonyl)pyrimidine-5-carboxylate (5.55 g, 82%); ¹H NMR (400 MHz) 1.87 (1H, m), 2.04 (1H, m), 2.36 (4H, m), 3.47 (3H, s), 3.91 (3H, s), 4.31 (1H, m), 9.23 (1H, s); m/z MH⁺=271; HPLC tR=2.20 min.

### Intermediate 4

### Methyl 4-cyclobutyl-2-(methylthio)pyrimidine-5-carboxylate

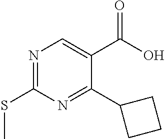

2-Methyl-2-thiopseudourea sulfate (1.93 g, 13.9 mmol) was added to (Z)-methyl 2-(cyclobutanecarbonyl)-3-(dimethylamino)acrylate (Intermediate 5, 2.6 g, 12.3 mmol) and sodium acetate (4.24 g, 51.7 mmol) in DMF (10 mL) at 20° C. The resulting solution was stirred at 80° C. for 2 h. Water was added to the cooled solution. The reaction mixture was diluted with EtOAc and washed with water. The organic layer was dried over MgSO₄, filtered and evaporated to afford crude product. The crude product was purified by FCC, elution gradient 5 to 30% EtOAc in isohexane to afford methyl 4-cyclobutyl-2-(methylthio)pyrimidine-5-carboxylate (1.30 g, 44%) as a white solid; ¹H NMR (400 MHz, CDCl₃) 1.86-1.94 (1H, m), 2.00-2.10 (1H, m), 2.26-2.35 (2H, m), 2.41-2.51 (2H, m), 2.65 (3H, s), 3.90 (3H, s), 4.35 (1H, qn), 8.86 (1H, s); m/z MH⁺=239; HPLC t_{R}=2.75 min.

### Intermediate 5

### Methyl 2-(cyclobutanecarbonyl)-3-(dimethylamino)acrylate

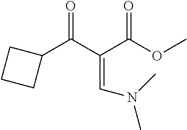

DMF dimethyl acetal (5.62 mL, 42.3 mmol) was added in one portion to methyl 3-cyclobutyl-3-oxopropanoate (5.5 g, 35.2 mmol) in dioxane (50 mL) at RT. The resulting solution was stirred at 100° C. for 4 h. The reaction mixture was concentrated and the resulting crude product was purified by FCC, eluting with 59-80% EtOAc/isohexane, to afford the title compound (4.60 g, 62%); ¹H NMR (400 MHz, CDCl₃) 1.72-1.82 (1H, m), 1.85-1.97 (1H, m), 2.06-2.13 (2H, m), 2.18-2.29 (2H, m), 3.02 (6H, s), 3.68-3.75 (1H, m), 3.73 (3H, s), 7.62 (1H, s); m/z [M+Na]⁺=234; HPLC t_{R}=1.42 min.

### Example 2

### 4-Cyclobutyl-N-(5-hydroxy-2-adamantyl)-2-[[(3R)-tetrahydrofuran-3-yl]amino]pyrimidine-5-carboxamide

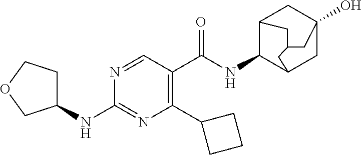

The title compound was prepared in a similar manner to Example 1 from (1S,4R)-4-aminoadamantan-1-ol.HCl and Intermediate 6; ¹H NMR (400 MHz) 1.31 (2H, d), 1.61 (4H, d), 1.67-1.80 (3H, m), 1.95 (7H, m), 2.12 (3H, m), 2.28 (2H, m), 3.54 (1H, m), 3.72 (1H, m), 3.87 (4H, m), 4.41 (2H, s), 7.56 (1H, d), 7.96 (1H, d), 8.15 (1H, s); m/z MH⁺=413; HPLC tR=1.56 min.

### Intermediate 6

### 4-Cyclobutyl-2-[[(3R)-oxolan-3-yl]amino]pyrimidine-5-carboxylic acid

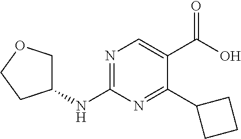

2 M aq. NaOH (1.48 mL, 2.96 mmol) was added to (R)-methyl 4-cyclobutyl-2-(tetrahydrofuran-3-ylamino)pyrimidine-5-carboxylate (Intermediate 7, 410 mg, 1.48 mmol) in MeOH (10 mL) and the resulting solution was stirred at RT for 16 h followed by heating at 60° C. for 6 h. The reaction mixture was concentrated, diluted with water and washed with ether. The aqueous layer was acidified with 1 M aq. citric acid. The resulting precipitate was collected by filtration, washed with water and dried under vacuum at 50° C. to afford the title compound (290 mg, 67%) as a white solid; ¹H NMR (400 MHz) 1.75-1.90 (1H, m), 1.92-2.10 (2H, m), 2.24-2.45 (5H, m), 3.58-3.68 (1H, m), 3.72-3.80 (1H, m), 3.85-4.05 (2H, m), 4.30-4.42 (1H, m), 4.45-4.60 (1H, m), 7.97-8.15 (1H, m), 8.63-8.72 (1H, m), 8.85 (1H, s), 12.64 (1H, s); m/z MH⁺=264; HPLC tR=1.48 min.

### Intermediate 7

### Methyl 4-cyclobutyl-2-[[(3R)-oxolan-3-yl]amino]pyrimidine-5-carboxylate

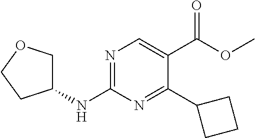

(R)-Tetrahydrofuran-3-amine 4-methylbenzenesulfonate (576 mg, 2.22 mmol) was added to methyl 4-cyclobutyl-2-(methylsulfonyl)pyrimidine-5-carboxylate (Intermediate 3, 400 mg, 1.48 mmol) and DIPEA (0.516 mL, 2.96 mmol) in THF (10 mL) and the resulting solution was stirred at RT for 16 h. The reaction mixture was evaporated to dryness, the residue was taken up in DCM and washed sequentially with 1 N aq. citric acid, sat. aq. NaHCO₃ and sat. brine. The organic layer was dried over MgSO₄, filtered and evaporated to afford the title compound (410 mg, 100%); ¹H NMR (400 MHz) 1.75-1.77 (1H, m), 1.80-1.87 (1H, m), 2.05-2.19 (3H, m), 2.20-2.38 (3H, d), 3.50-3.58 (1H, m), 3.62-3.72 (1H, m), 3.70 (3H, s), 3.77-3.84 (1H, m), 4.09-4.18 (1H, m), 4.21-4.30 (1H, m), 4.40-4.50 (1H, m), 8.00-8.12 (1H, m), 8.55-8.62 (1H, m); m/z MH⁺=278; HPLC tR=2.00 min.

### Example 3

### 2-[(1-Acetyl-4-piperidyl)amino]-4-cyclopentyl-N-(5-hydroxy-2-adamantyl)pyrimidine-5-carboxamide

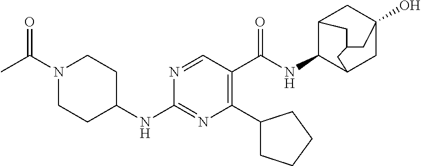

1-Acetylpiperidin-4-amine (339 mg, 2.38 mmol) was added to 4-cyclopentyl-N-(5-hydroxy-2-adamantyl)-2-methylsulfonylpyrimidine-5-carboxamide (400 mg, 0.95 mmol) in butyronitrile (3 mL). The reaction mixture was sealed into a microwave tube. The reaction was heated to 150° C. for 45 minutes in the microwave reactor and cooled to RT. The reaction mixture was evaporated to dryness, redissolved in DCM and washed with water. The organic layer was dried by passing down a isolute phase separator and evaporated to afford a pale yellow solid (459 mg). The crude product was purified by preparative HPLC using decreasingly polar mixtures of water (containing 0.5% NH₃) and MeCN as eluents to afford the title compound (105 mg, 23%) as a cream solid; ¹H NMR (400 MHz) 1.36 (5H, m), 1.58 (6H, m), 1.80 (12H, m), 2.00 (5H, m), 2.71 (1H, m), 3.12 (1H, t), 3.42 (1H, m), 3.79 (1H, d), 3.91 (2H, m), 4.25 (1H, d), 4.39 (1H, s), 7.25 (1H, s), 8.02 (1H, d), 8.13 (1H, s); m/z MH⁺=482; HPLC tR=1.52 min.

### Intermediate 8

### 4-Cyclopentyl-N-(5-hydroxy-2-adamantyl)-2-methylsulfonylpyrimidine-5-carboxamide

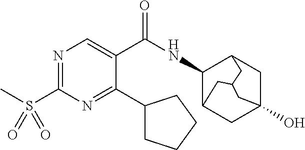

The title compound was prepared from Intermediate 9 in a similar manner to the synthesis of Intermediate 3; ¹H NMR (400 MHz, CDCl₃) 1.57-1.63 (2H, m), 1.69-1.99 (15H, m), 2.04-2.09 (2H, m), 2.17-2.23 (1H, m), 2.27-2.33 (2H, m), 3.30 (3H, s), 3.57 (1H, qn), 4.23-4.27 (1H, m), 6.43 (1H, d), 8.72 (1H, s); m/z MH⁺=420; HPLC t_{R}=1.75 min.

### Intermediate 9

### 4-Cyclopentyl-N-(5-hydroxy-2-adamantyl)-2-methylsulfanylpyrimidine-5-carboxamide

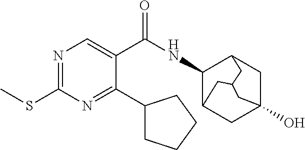

The title compound was prepared from Intermediate 10 in a similar manner to the synthesis of Example 1; ¹H NMR (400 MHz, CDCl₃) 1.35-1.42 (1H, m), 1.58-1.62 (2H, m), 1.65-1.72 (4H, m), 1.79-2.01 (12H, m), 2.16-2.21 (1H, m), 2.24-2.27 (2H, m), 2.56 (3H, s), 3.51 (1H, quintet), 4.18-4.23 (1H, m), 5.92 (1H, d), 8.42 (1H, s); m/z MH⁺=388; HPLC t_{R}=2.20 min.

### Intermediate 10

### 4-Cyclopentyl-2-(methylthio)pyrimidine-5-carboxylic acid

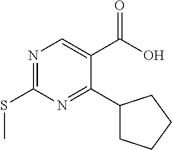

The title compound was prepared from Intermediate 11 in a similar manner to the synthesis of Intermediate 1; ¹H NMR (400 MHz, CDCl₃) 1.68-1.75 (2H, m), 1.81-1.96 (4H, m), 2.00-2.10 (2H, m), 2.61 (3H, s), 4.13 (1H, qn), 9.00 (1H, s), 11.21 (1H, br s); m/z MH⁺=239; HPLC t_{R}=1.19 min.

### Intermediate 11

### Methyl 4-cyclopentyl-2-(methylthio)pyrimidine-5-carboxylate

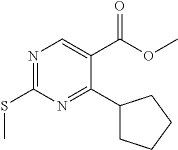

The title compound was prepared from Intermediate 12 in a similar manner to the synthesis of Intermediate 4; ¹H NMR (400 MHz, CDCl₃) 1.67-1.72 (2H, m), 1.79-1.92 (4H, m), 1.99-2.05 (2H, m), 2.58 (3H, s), 3.91 (3H, s), 3.99-4.09 (1H, m), 8.85 (1H, s); m/z MH⁺=253; HPLC t_{R}=3.04 min.

### Intermediate 12

### Methyl 2-(cyclopentanecarbonyl)-3-(dimethylamino)acrylate

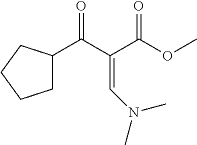

DMF dimethyl acetal (3.28 mL, 24.7 mmol) was added in one portion to methyl 3-cyclopentyl-3-oxopropanoate (3.50 g, 20.6 mmol) in 1,4-dioxane (40 mL) at RT. The resulting solution was stirred at 100° C. for 4 h. The reaction mixture was evaporated to afford crude product. The crude product was purified by FCC, elution gradient 50 to 80% EtOAc in isohexane, to afford the title product (4.50 g, 97%) as a yellow oil; ¹H NMR (400 MHz) 1.45-1.73 (8H, m), 2.81-2.86 (1H, m), 2.95 (6H, s), 3.62 (3H, s), 7.57 (1H, s); m/z MH⁺=226; HPLC t_{R}=1.66 min.

The following examples were made in a similar manner to Example 3 from the appropriate amine and 4-cyclopentyl-N-(5-hydroxy-2-adamantyl)-2-methylsulfonylpyrimidine-5-carboxamide (Intermediate 8):

| Structure | Ex no. | name | ¹H NMR | LCMS |
|---|---|---|---|---|
| 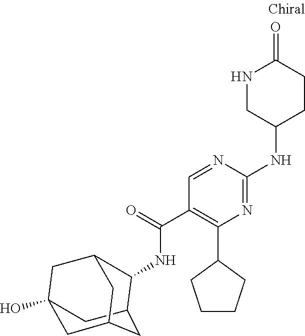 | 4 | 4-cyclopentyl-N- (5-hydroxy-2- adamantyl)-2- [[(3R)-6-oxo-3- piperidyl]amino] pyrimidine-5- carboxamide | ¹H NMR (400 MHz) 1.31 (2H, d), 1.59 (6H, m), 1.76 (9H, m), 1.76 (9H, m), 2.01 (8H, m), 3.33 (2H, m), 3.42 (1H, m), 3.69 (1H, m), 3.89 (1H, m), 4.37 (1H, s), 7.31 (1H, s), 7.63 (1H, s), 8.04 (1H, d), 8.13 (1H, s) | m/z MH⁺ = 454; HPLC tR = 1.54 min. |
| 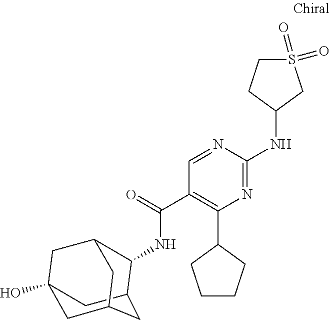 | 5 | 4-cyclopentyl-2- [(1,1- dioxothiolan-3- yl)amino]-N-(5- hydroxy-2- adamantyl) pyrimidine-5- carboxamide | ¹H NMR (400 MHz) 1.32 (2H, d), 1.60 (6H, m), 1.86 (13H, m), 2.21 (1H, m), 2.45 (1H, m), 2.99 (1H, m), 3.18 (1H, m), 3.42 (3H, m), 3.90 (1H, m), 4.38 (1H, s), 4.59 (1H, m), 7.71 (1H, d), 8.09 (1H, d), 8.18 (1H, s) | m/z MH⁺ = 475; HPLC tR = 1.67 min. |
| 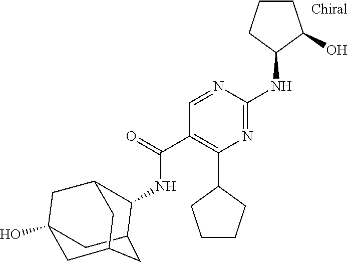 | 6 | 4-cyclopentyl-N- (5-hydroxy-2- adamantyl)-2- [[(1S,2R)-2- hydroxycyclo- pentyl]amino] pyrimidine-5- carboxamide | ¹H NMR (400 MHz) 1.31 (2H, d), 1.54 (9H, m), 1.76 (10H, m), 1.96 (6H, m), 3.42 (1H, m), 3.89 (1H, m), 3.97 (1H, m), 4.06 (1H, s), 4.38 (1H, s), 4.68 (1H, s), 6.44 (1H, s), 8.03 (1H, d), 8.13 (1H, s) | m/z MH⁺ = 441; HPLC tR = 1.88 min. |
| 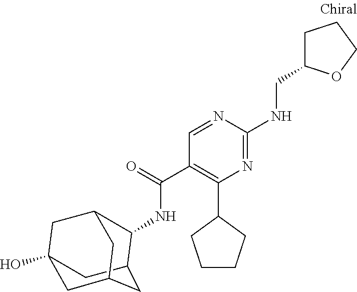 | 7 | 4-cyclopentyl-N- (5-hydroxy-2- adamantyl)-2- [[(2S)- tetrahydrofuran- 2-yl] methylamino] pyrimidine-5- carboxamide | ¹H NMR (400 MHz) 1.31 (2H, m), 1.59 (7H, m), 1.85 (16H, m), 3.28 (1H, m), 3.40 (2H, m), 3.60 (1H, q), 3.75 (1H, q), 3.88 (1H, m), 3.98 (1H, m), 4.38 (1H, s), 7.21 (1H, s), 8.02 (1H, d), 8.11 (1H, s) | m/z MH⁺ = 441; HPLC tR = 1.94 min. |
| 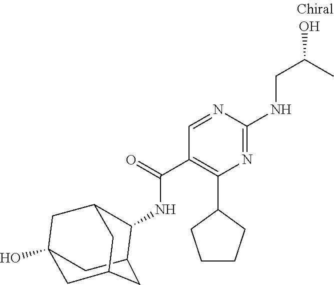 | 8 | 4-cyclopentyl-N- (5-hydroxy-2- adamantyl)-2- [[(2R)-2- hydroxypropyl] amino] pyrimidine-5- carboxamide | 1H NMR (400 MHz) 1.11 (3H, d), 1.38 (2H, d), 1.64 (6H, m), 1.83 (8H, m), 2.03 (5H, m), 3.30 (2H, m), 3.49 (1H, m), 3.85 (1H, m), 3.95 (1H, m), 4.45 (1H, s), 4.71 (1H, d), 7.16 (1H, s), 8.08 (1H, d), 8.18 (1H, s) | m/z M+ = 415; HPLC tR = 1.62 min. |
| 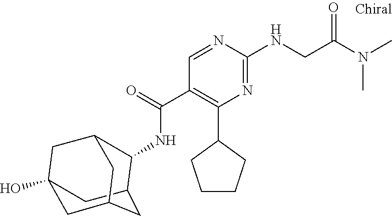 | 9 | 4-cyclopentyl-2- [[2- (dimethylamino)- 2-oxo- ethyl]amino]-N- (5-hydroxy-2- adamantyl) pyrimidine-5- carboxamide | ¹H NMR (400 MHz) 1.31 (2H, d), 1.58 (6H, m), 1.76 (8H, m), 1.96 (5H, m), 2.82 (3H, s), 3.01 (3H, s), 3.42 (1H, m), 3.89 (1H, m), 4.07 (2H, d), 4.37 (1H, s), 7.09 (1H, s), 8.07 (1H, d), 8.12 (1H, s) | m/z MH⁺ = 442; HPLC tR = 1.58 min. |
| 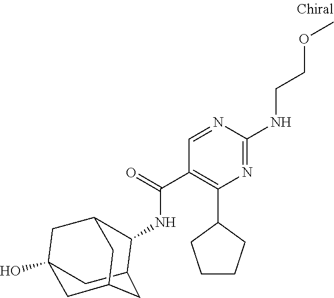 | 10 | 4-cyclopentyl-N- (5-hydroxy-2- adamantyl)-2-(2- methoxyethyl- amino) pyrimidine-5- carboxamide | ¹H NMR (400 MHz) 1.31 (2H, d), 1.58 (6H, m), 1.76 (8H, m), 1.97 (5H, m), 3.26 (7H, m), 3.41 (1H, m), 3.89 (1H, m), 4.38 (1H, s), 7.19 (1H, s), 8.02 (1H, d), 8.12 (1H, s) | m/z MH⁺ = 415; HPLC tR = 1.83 min. |
| 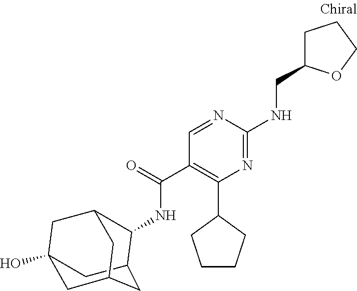 | 11 | 4-cyclopentyl-N- (5-hydroxy-2- adamantyl)-2- [[(2R)- tetrahydrofuran- 2-yl] methylamino] pyrimidine-5- carboxamide | ¹H NMR (400 MHz) 1.31 (2H, d), 1.57 (7H, m), 1.85 (16H, m), 3.27 (1H, m), 3.40 (2H, m), 3.60 (1H, q), 3.75 (1H, q), 3.88 (1H, m), 3.98 (1H, m), 4.37 (1H, s), 7.21 (1H, s), 8.01 (1H, d), 8.11 (1H, s) | m/z MH⁺ = 441; HPLC tR = 1.95 min. |
| 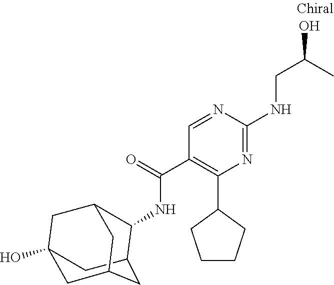 | 12 | 4-cyclopentyl-N- (5-hydroxy-2- adamantyl)-2- [[(2S)-2- hydroxypropyl] amino] pyrimidine-5- carboxamide | ¹H NMR (400 MHz) 1.04 (3H, d), 1.31 (2H, d), 1.57 (6H, m), 1.76 (8H, m), 1.97 (5H, m), 3.23 (2H, m), 3.42 (1H, m), 3.78 (1H, m), 3.88 (1H, m), 4.37 (1H, s), 4.64 (1H, d), 7.08 (1H, s), 8.01 (1H, d), 8.12 (1H, s) | m/z MH⁺ = 415; HPLC tR = 1.62 min. |
| 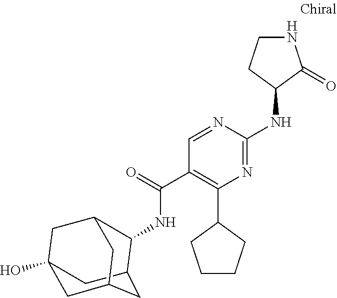 | 13 | 4-cyclopentyl-N- (5-hydroxy-2- adamantyl)-2- [[(3S)-2- oxopyrrolidin-3- yl]amino] pyrimidine-5- carboxamide | ¹H NMR (400 MHz) 1.38 (2H, d), 1.65 (6H, m), 1.82 (8H, m), 2.06 (6H, m), 2.38 (1H, m), 3.28 (3H, m), 3.47 (1H, m), 3.96 (1H, m), 4.45 (1H, s), 7.40 (1H, d), 7.77 (1H, s), 8.13 (1H, d), 8.18 (1H, s) | m/z MH⁺ = 440; HPLC tR = 1.34 min. |
| 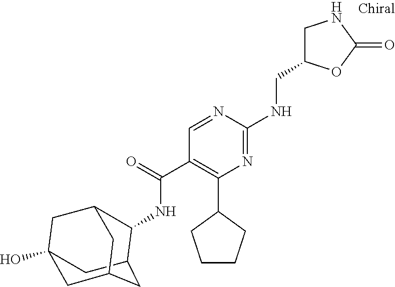 | 14 | 4-cyclopentyl-N- (5-hydroxy-2- adamantyl)-2- [[(3R)-5- oxopyrrolidin-3- yl]methylamino] pyrimidine-5- carboxamide | ¹H NMR (400 MHz) 1.31 (2H, d), 1.58 (7H, m), 1.76 (9H, m), 1.96 (7H, m), 2.21 (1H, m), 2.65 (1H, m), 3.01 (1H, m), 3.42 (1H, m), 3.88 (1H, m), 4.38 (1H, s), 7.45 (2H, s), 8.03 (1H, d), 8.12 (1H, s) | m/z MH⁺ = 454; HPLC tR = 1.49 min. |
| 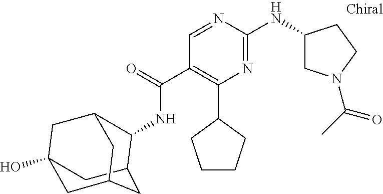 | 15 (from Inter- medi- ate 13) | 2-[[(3R)-1- acetylpyrrolidin- 3-yl]amino]-4- cyclopentyl-N- (5-hydroxy-2- adamantyl) pyrimidine-5- carboxamide | ¹H NMR (400 MHz) 1.31 (2H, d), 1.59 (6H, m), 1.77 (8H, m), 1.96 (9H, m), 2.13 (1H, m), 3.28 (2H, m), 3.44 (2H, m), 3.65 (1H, m), 3.89 (1H, m), 4.36 (2H, m), 7.54 (1H, d), 8.04 (1H, d), 8.16 (1H, d) | m/z MH⁺ = 468; HPLC tR = 1.62 min. |
| 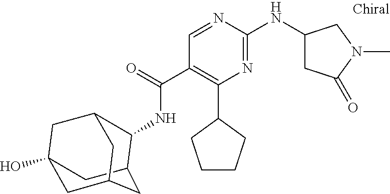 | 16 | 4-cyclopentyl-N- (5-hydroxy-2- adamantyl)-2- [(1-methyl-5- oxo-pyrrolidin-3- yl)amino] pyrimidine-5- carboxamide | ¹H NMR (400 MHz) 1.29-1.35 (2H, m), 1.53-1.64 (7H, m), 1.67-2.09 (12H, m), 2.31 (1H, dd), 2.61 (1H, dd), 2.71 (3H, s), 3.24 (1H, dd), 3.38-3.46 (1H, m), 3.67 (1H, dd), 3.85-3.92 (1H, m), 4.34-4.44 (1H, m), 4.39 (1H, s), 7.59-7.65 (1H, m), 8.01-8.08 (1H, d), 8.16 (1H, s) | m/z MH⁺ = 454; HPLC tR = 1.50 min |
| 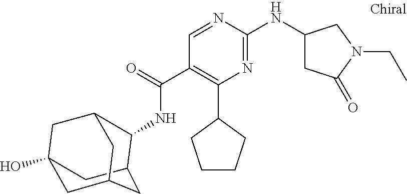 | 17 | 4-cyclopentyl-2- [(1-ethyl-5-oxo- pyrrolidin-3- yl)amino]-N-(5- hydroxy-2- adamantyl) pyrimidine- 5-carboxamide | ¹H NMR (400 MHz) 1.00 (3H, t), 1.31-1.39 (2H, m), 1.53-1.64 (7H, m), 1.68-2.08 (12H, m), 2.35 (1H, dd), 2.63 (1H, dd), 3.16-3.30 (3H, m), 3.39-3.46 (1H, m), 3.69 (1H, dd), 3.87-3.91 (1H, m), 4.38 (1H, s), 4.39-4.48 (1H, m), 7.60-7.68 (1H, m), 8.04-8.08 (1H, m), 8.16 (1H, s) | m/z MH⁺ = 468; HPLC tR = 1.61 min |
| 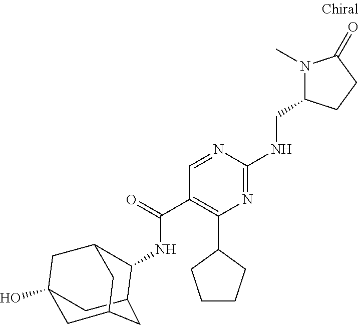 | 18 (from Inter- medi- ate 15) | 4-cyclopentyl-N- (5-hydroxy-2- adamantyl)-2- [[(2R)-1-methyl- 5-oxo-pyrrolidin- 2-yl] methylamino] pyrimidine-5- carboxamide | 1H NMR (400 MHz, DMSO) δ 1.31 (2H, d), 1.49-1.65 (6H, m), 1.65-2.04 (15H, m), 2.06-2.15 (1H, m), 2.15-2.28 (1H, m), 2.75 (3H, s), 3.34-3.50 (2H, m), 3.62 (2H, dd), 3.84-3.93 (1H, m), 4.37 (1H, s), 7.46 (1H, s), 8.05 (1H, d), 8.13 (1H, s). | m/z MH⁺ = 468; HPLC tR = 1.53 min. |

### Intermediate 13

### 1-[(3R)-3-aminopyrrolidin-1-yl]ethanone

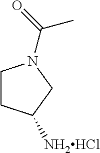

Tert-butyl N-[(3R)-1-acetylpyrrolidin-3-yl]carbamate (Intermediate 14, 90 g, 0.39 mol) was added to HCl/MeOH (1 L) at 0° C. and the reaction mixture was stirred for 3 h at RT. The reaction mixture was concentrated and the residue was washed with diethyl ether to afford the title compound (55 g, 85%); ¹H NMR (300 MHz, MeOD) 2.12 (3H, m), 2.18-2.48 (2H, m), 3.56-3.79 (4H, m), 3.92-3.96 (1H, m); no mass ion; HPLC tR=2.78 min.

### Intermediate 14

### Tert-butyl N-[(3R)-1-acetylpyrrolidin-3-yl]carbamate

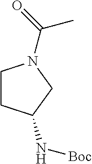

To a solution of tert-butyl N-[(3R)-pyrrolidin-3-yl]carbamate (90 g, 0.483 mol) in DCM (0.8 L) was added NEt₃ (97.8 g, 0.966 mol) and acetyl chloride (75.8 g, 0.966 mol) at 0° C. The reaction mixture was stirred for 3 h, then was quenched with sat. brine and the aqueous layer was extracted with DCM. The combined organic layers were dried over MgSO₄, filtered and concentrated to afford the title compound (110 g, 99%); no mass ion; HPLC tR=0.79 min.

### Intermediate 15

### (5R)-5-(aminomethyl)-1-methyl-pyrrolidin-2-one

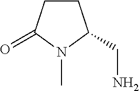

To a solution of diazonio-[[(2R)-1-methyl-5-oxopyrrolidin-2-yl]methyl]azanide (Intermediate 16, 80 g, 0.519 mol) in MeOH (800 mL) was added 10% Pd/C (8 g). The reaction was stirred at RT for 14 h. The mixture was filtered and the filtrate was concentrated. The residue was purified by FCC, eluting with 2.5-10% MeOH/DCM to afford the title compound (42 g, 64%); ¹H NMR (400 MHz, CDCl₃) 1.65-2.28 (4H, m), 2.66-2.76 (5H, m), 3.37-3.38 (1H, m); m/z MH⁺=129.

### Intermediate 16

### Diazonio-[[(2R)-1-methyl-5-oxopyrrolidin-2-yl]methyl]azanide

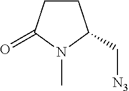

To a solution of [(2R)-1-methyl-5-oxopyrrolidin-2-yl]methyl methanesulfonate (Intermediate 17, 160 g, 0.773 mol) in 10:1 THF:H₂O (1.6 L) was added NaN₃ (100 g, 1.55 mol) at RT. The reaction was stirred at 80° C. for 16 h, then the reaction mixture was adjusted to pH 11 with 2 N aq. NaOH and extracted with DCM. The organic phase was dried over Na₂SO₄ and concentrated. The residue was purified by FCC, eluting with 30:1 DCM:MeOH to give the title compound (80 g, 67%) as a yellow oil; m/z MH⁺=155.

### Intermediate 17

### [(2R)-1-Methyl-5-oxopyrrolidin-2-yl]methyl methanesulfonate

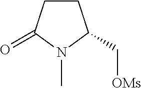

To a solution of (5R)-5-(hydroxymethyl)-1-methylpyrrolidin-2-one (Intermediate 18, 130 g, 1.01 mol) and NEt₃ (202 g, 2.02 mol) in DCM (1.3 L) was added MsCl (116 g, 1.01 mol) dropwise at 0° C. over 30 minutes. The reaction mixture was stirred at RT for 3 h, then the reaction mixture was diluted with water and extracted with DCM. The organic layer was dried over Na₂SO₄ and concentrated to afford the title compound (160 g, 76%); m/z M⁺=207.

### Intermediate 18

### (5R)-5-(Hydroxymethyl)-1-methylpyrrolidin-2-one

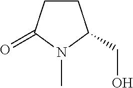

To a solution of methyl (2R)-1-methyl-5-oxopyrrolidine-2-carboxylate (Intermediate 19, 180 g, 1.15 mol) in MeOH (1.8 L) was added NaBH₄ (87 g, 2.29 mol) portionwise at 0° C. over 1 h. The reaction mixture was stirred at RT for 3 h, then the reaction mixture was concentrated and the residue was diluted with 100 mL of 1 N aq. HCl and extracted with DCM. The organic layer was dried over Na₂SO₄ and concentrated to afford the title compound (130 g, 88%); ¹H NMR (400 MHz, CDCl₃) 1.58-2.08 (4H, m), 2.44-2.47 (3H, m), 3.18-3.23 (2H, m), 3.40-3.44 (1H, m), 4.35 (1H, s).

### Intermediate 19

### Methyl (2R)-1-methyl-5-oxopyrrolidine-2-carboxylate

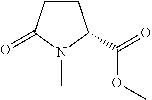

To a solution of methyl (2R)-5-oxopyrrolidine-2-carboxylate (Intermediate 20, 260 g, 1.82 mol) and K₂CO₃ (501 g, 3.64 mol) in CH₃CN (2.5 L) was added CH₃I (775 g, 5.46 mol) dropwise at RT. The reaction was stirred at 80° C. for 72 h, then the mixture was filtered and the filtrate was concentrated. The residue was diluted with 200 mL of water and extracted with DCM. The organic phase was dried over Na₂SO₄ and concentrated to afford the title compound (180 g, 63%) as a yellow oil; ¹H NMR (400 MHz, CDCl₃) 1.98-2.01 (1H, m), 2.27-2.37 (3H, m), 2.75 (3H, s), 3.67 (3H, s), 4.03-4.06 (1H, m); m/z MH⁺=158.

### Intermediate 20

### Methyl (2R)-5-oxopyrrolidine-2-carboxylate

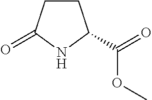

To a solution of (2R)-5-oxopyrrolidine-2-carboxylic acid (250 g, 1.92 mol) in MeOH (2 L) was added SOCl₂ (210 mL, 2.51 mol) dropwise at 0° C. over 1 h. The reaction mixture was stirred at RT for 16 h. The reaction mixture was concentrated to give the title compound (260 g, 94%); ¹H NMR (400 MHz, CDCl₃) 2.19-2.49 (4H, m), 3.62-3.80 (4H, m), 4.24-4.26 (1H, m), 6.73 (1H, s).

### Example 19

### 4-cyclopentyl-N-(5-hydroxy-2-adamantyl)-2-[[(3S)-5-oxopyrrolidin-3-yl]methylamino]pyrimidine-5-carboxamide

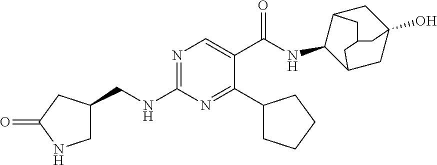

(S)-4-(Aminomethyl)pyrrolidin-2-one (Intermediate 21, 163 mg, 1.43 mmol) was added to 4-cyclopentyl-N-(5-hydroxy-2-adamantyl)-2-methylsulfonylpyrimidine-5-carboxamide (Intermediate 8, 600 mg, 1.43 mmol) in butyronitrile (3 mL). The reaction mixture was heated at 150° C. for 1 h in the microwave reactor and cooled to RT. The reaction mixture was concentrated, the residue was taken up in DCM, washed with water and the organic layer was dried by passing through an isolute phase separator and concentrated. The crude product was purified by FCC, eluting with 0-40% MeOH/EtOAc, to afford the title compound (500 mg, 77%) as a white solid; ¹H NMR (400 MHz) 1.30-1.33 (2H, m), 1.54-1.63 (6H, m), 1.71-1.93 (8H, m), 1.96-2.12 (6H, m), 2.18-2.24 (1H, m), 2.64-2.73 (1H, m), 3.02-3.09 (1H, m), 3.33-3.44 (3H, m), 3.45-3.52 (1H, m), 3.88-3.94 (1H, m), 4.41 (1H, s), 7.44-7.52 (2H, m), 8.03-8.06 (1H, m), 8.12 (1H, s); m/z MH⁺=454; HPLC tR=1.39 min.

### Intermediate 21

### (4S)-4-(Aminomethyl)pyrrolidin-2-one

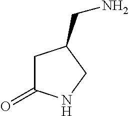

Na (9.0 g, 0.39 mol) was added to liquid NH₃ (250 mL) portionwise at −65° C. over 15 minutes. (4S)-4-(aminomethyl)-1-[(1R)-1-phenylethyl]pyrrolidin-2-one (Intermediate 22, 28.5 g, 0.13 mol) in THF (250 ml) was added dropwise at −65° C. over 1 h. The mixture was stirred at −60° C. for 2 h and then the temperature was warmed to 0° C. slowly and stirred for 4 h. The reaction was quenched with MeOH and diluted with DCM. The mixture was purified by FCC, eluting with 2.5-20% MeOH/DCM to afford the title compound (7.9 g, 53%); ¹H NMR (400 MHz, CDCl₃) 1.32 (2H, s), 2.00-2.06 (1H, m), 2.36-2.52 (2H, m), 2.70-2.79 (2H, m), 3.07-3.12 (1H, m), 3.43-3.51 (1H, m), 6.02 (1H, s); m/z MH⁺=115; HPLC tR=4.87 min.

### Intermediate 22

### (4S)-4-(Aminomethyl)-1-[(1R)-1-phenylethyl]pyrrolidin-2-one

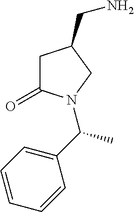

To a solution of (3R)-5-oxo-1-[(1R)-1-phenylethyl]pyrrolidine-3-carbonitrile (Intermediate 23, 6.0 g, 28.0 mmol), NH₃.H₂O (30 mL) and Raney-Ni (5.0 g) in MeOH (170 mL) was bubbled H₂ gas and then the mixture was stirred under H₂ atmosphere (40 psi) at RT for 2 h. The mixture was filtered and the filtrate was concentrated to afford the title compound (6.1 g, 100%) as a colourless oil which was used without further purification.

### Intermediate 23

### (3R)-5-oxo-1-[(1R)-1-phenylethyl]pyrrolidine-3-carbonitrile

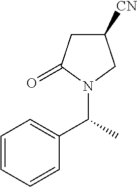

To a solution of (3R)-5-oxo-1-[(1R)-1-phenylethyl]pyrrolidine-3-carboxamide (Intermediate 24, crude, 0.99 mol if pure) in DCM (2.5 L) was added NaHCO₃ (249.5 g, 2.97 mol) and trifluoroacetic acid anhydride (415.8 g, 1.98 mol) at 0° C. The reaction mixture was stirred at 0° C. for 4 h and then warmed to RT and stirred for 14 h. The mixture was filtered and the filtrate was concentrated, then was purified by FCC, eluting with 20-50% EtOAc/isohexane to afford the title compound (81 g, 38%) as a white solid; ¹H NMR (400 MHz, CDCl₃) 1.57 (3H, d), 2.72-2.83 (2H, m), 3.12-3.20 (1H, m), 3.30-3.34 (1H, m), 3.52-3.58 (1H, m), 5.47-5.54 (1H, m), 7.26-7.37 (5H, m).

### Intermediate 24

### (3R)-5-Oxo-1-[(1R)-1-phenylethyl]pyrrolidine-3-carboxamide

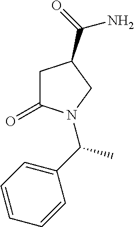

A solution of (3R)-5-oxo-1-[(1R)-1-phenylethyl]pyrrolidine-3-carboxylic acid (Intermediate 25, 230 g, 0.99 mol) and carbonyl diimidazole (193 g, 1.18 mol) in THF (1.5 L) was stirred at 0° C. for 15 minutes. A sat. solution of NH₃/THF (1.0 L) was added at 0° C. over 30 minutes. The reaction mixture was stirred at 0° C. for 4 h, then was filtered and the filtrate was concentrated to give the title compound as a crude product that was used directly without further purification.

### Intermediate 25

### (3R)-5-Oxo-1-[(1R)-1-phenylethyl]pyrrolidine-3-carboxylic acid

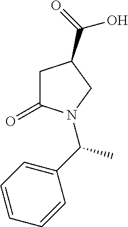

A mixture of 2-methylidenebutanedioic acid (500 g, 3.85 mol) and (R)-1-phenylethanamine (465.4 g, 3.85 mol) in 1,3-dimethyl-2-imidazolidinone (700 mL) was stirred at 120° C. for 14 h. Water (2 L) was added and the mixture was filtered. The collected solid was recrystallised 3 times from propan-2-ol to afford the title compound (264 g, 30%).

### Example 20

### N-(5-hydroxy-2-adamantyl)-4-isopropyl-2-[[(3S)-tetrahydrofuran-3-yl]amino]pyrimidine-5-carboxamide

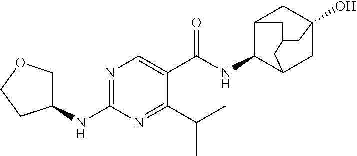

(S)-Tetrahydrofuran-3-amine hydrochloride (229 mg, 1.85 mmol), DIPEA (0.392 mL, 2.25 mmol) and N-(5-hydroxy-2-adamantyl)-2-methylsulfonyl-4-propan-2-ylpyrimidine-5-carboxamide (Intermediate 26, 442.9 mg, 1.13 mmol) were suspended in THF (20 mL) and the reaction mixture was heated at 100° C. for 12 h in the microwave reactor and then cooled to RT. The reaction mixture was concentrated and taken up in DCM, then was washed sequentially with sat. aq. NaHCO₃ and water. The organic layer was dried over MgSO₄, filtered and concentrated. The resultant crude product was purified by preparative HPLC to afford the title compound (185 mg, 41%) as a white solid; ¹H NMR (400 MHz) 1.13 (6H, d), 1.31 (2H, d), 1.58-1.63 (4H, m), 1.70 (2H, d), 1.86-1.95 (2H, m), 1.98 (1H, s), 2.03 (2H, s), 2.11-2.16 (1H, m), 3.52 (1H, q), 3.68-3.73 (1H, m), 3.82 (1H, q), 3.86-3.91 (2H, m), 4.34-4.41 (2H, m), 7.50 (1H, br s), 8.05 (1H, d), 8.16 (1H, s); m/z MH⁺=401; HPLC tR=1.58 min.

### Intermediate 26

### N-(5-hydroxy-2-adamantyl)-2-methylsulfonyl-4-propan-2-ylpyrimidine-5-carboxamide

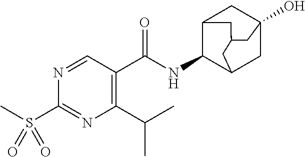

3-Chlorobenzoperoxoic acid (1.82 g, 7.38 mmol) was added to N-(5-hydroxy-2-adamantyl)-2-methylsulfanyl-4-propan-2-ylpyrimidine-5-carboxamide (Intermediate 27, 1.05 g, 2.90 mmol) in DCM (200 mL) at 0° C. The resulting solution was stirred at RT for 24 h. The reaction mixture was diluted with DCM, and washed sequentially with sat. aq. NaHCO₃ and sat. brine. The organic layer was concentrated to afford the title compound (1.08 g, 94%); ¹H NMR (400 MHz) 1.24 (6H, d), 1.36 (2H, d), 1.62-1.66 (4H, m), 1.72-1.75 (2H, m), 1.86 (2H, d), 1.99 (1H, s), 2.07 (2H, s), 3.26-3.34 (2H, m), 3.43 (3H, s), 3.99-4.03 (1H, m), 8.60 (1H, d), 8.88 (1H, s); m/z [M-H]⁻=392; HPLC tR=1.90 min.

### Intermediate 27

### N-(5-hydroxy-2-adamantyl)-2-methylsulfanyl-4-propan-2-ylpyrimidine-5-carboxamide

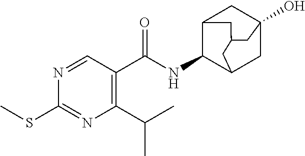

HATU (4.20 g, 11.05 mmol) was added to 4-isopropyl-2-(methylthio)pyrimidine-5-carboxylic acid (Intermediate 28, 1.89 g, 8.91 mmol) and DIPEA (3.10 mL, 17.8 mmol) in DMF (50 mL). The resulting solution was stirred at RT for 30 minutes. 4-Aminoadamantan-1-ol hydrochloride (2.21 g, 10.9 mmol) was added and stirred at RT overnight. The reaction mixture was concentrated, taken up in EtOAc (150 mL) and washed sequentially with sat. aq. NaHCO₃, water, and sat. brine. The organic layer was dried over MgSO₄, filtered and concentrated to afford crude product. The crude product was purified by FCC, eluting with 0-5% MeOH/DCM, to afford the title compound (2.67 g, 83%); ¹H NMR (400 MHz) 1.18 (6H, d), 1.33 (2H, d), 1.60 (3H, s), 1.64 (1H,$), 1.70-1.73 (2H, m), 1.89 (2H, d), 1.98 (1H, s), 2.04 (2H, s), 2.53 (3H, s), 3.27 (1H, q), 3.92-3.97 (1H, m), 4.40 (1H, s), 8.36 (1H, d), 8.43 (1H, s); m/z MH⁺=362; HPLC tR=1.89 min.

### Intermediate 28

### 4-Isopropyl-2-(methylthio)pyrimidine-5-carboxylic acid

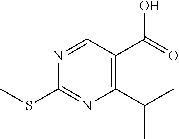

Ethyl 4-isopropyl-2-(methylthio)pyrimidine-5-carboxylate (Intermediate 29, 2.57 g, 10.7 mmol) was dissolved in MeOH (100 mL) and 21 mL of 2 M aq. NaOH was added. The resulting solution was stirred at RT for 4 h. The reaction mixture was concentrated, taken up in water and washed with EtOAc. The aqueous layer was acidified with citric acid, and the solid was filtered and dried to afford the title compound (1.89 g, 83%) as a white solid; ¹H NMR (400 MHz) 1.19 (6H, d), 2.55 (3H, s), 3.29 (1H, br s), 3.90-3.97 (1H, m), 8.86 (1H, s); m/z [M-H]⁻=211; HPLC tR=0.77 min.

### Intermediate 29

### Ethyl 4-isopropyl-2-(methylthio)pyrimidine-5-carboxylate

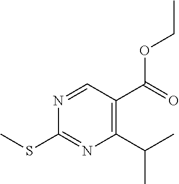

Methyl carbamimidothioate hemisulfate (46.7 g, 167 mmol) was added to (Z)-ethyl 2-((dimethylamino)methylene)-4-methyl-3-oxopentanoate (Intermediate 30, 35.9 g, 168 mmol) and sodium acetate (17.4 g, 212 mmol) in DMF (300 mL) at RT. The resulting solution was stirred at 80° C. for 4 h. The reaction mixture was concentrated and taken up in EtOAc (300 mL), then was washed with water. The organic layer was dried over MgSO₄, filtered and concentrated to afford crude product. Water (250 mL) was added and the mixture was stirred for an hour at RT to obtain a crystalline solid. The solid was filtered off and washed with water (100 mL) and then dried to afford the title compound (28.4 g, 70%) as a white solid; ¹H NMR (400 MHz) 1.20 (6H, d), 1.31 (3H, t), 2.56 (3H, s), 3.78-3.85 (1H, m), 4.31 (2H, q), 8.86 (1H, s); m/z MH⁺=241; HPLC tR=2.79 min.

### Intermediate 30

### Ethyl 2-((dimethylamino)methylene)-4-methyl-3-oxopentanoate

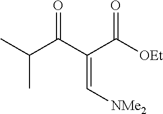

DMF dimethyl acetal (30.3 mL, 227.6 mmol) was added in one portion to ethyl 4-methyl-3-oxopentanoate (30 g, 189.6 mmol) in 1,4-dioxane (150 mL) at RT and the resulting solution was stirred at 100° C. for 4 h. The reaction mixture was evaporated to afford the title compound (36 g, 89%); ¹H NMR (400 MHz) 0.95 (6H, d), 1.20 (3H, t), 2.75-3.05 (6H, m), 3.10-3.17 (1H, m), 4.10 (2H, q), 7.55 (1H, s)

### Example 21

### N-(5-hydroxy-2-adamantyl)-4-isopropyl-2-[[(3R)-tetrahydrofuran-3-yl]amino]pyrimidine-5-carboxamide

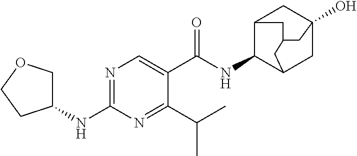

The title compound was prepared in a similar manner to Example 20 from N-(5-hydroxy-2-adamantyl)-2-methylsulfonyl-4-propan-2-ylpyrimidine-5-carboxamide (Intermediate 26) and (R)-tetrahydrofuran-3-amine 4-methylbenzenesulfonate; ¹H NMR (400 MHz) 1.13 (6H, d), 1.31 (2H, d), 1.60 (3H, s), 1.63 (1H, s), 1.69-1.72 (2H, m), 1.86-1.95 (2H, m), 1.98 (1H, s), 2.02 (2H, s), 2.09-2.18 (1H, m), 3.53 (1H, dd), 3.68-3.73 (1H, m), 3.82 (1H, q), 3.86-3.91 (2H, m), 4.34-4.39 (2H, m), 7.51 (1H, br s), 8.05 (1H, d), 8.16 (1H, s); m/z MH⁺=401; HPLC tR=1.58 min.

### Example 22

### N-(5-hydroxy-2-adamantyl)-2-[[(3R)-oxolan-3-yl]amino]-4-(trifluoromethyl)pyrimidine-5-carboxamide

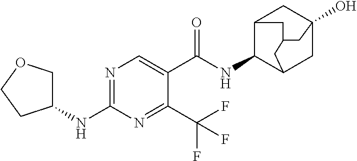

HATU (686 mg, 1.80 mmol) was added to (R)-2-(tetrahydrofuran-3-ylamino)-4-(trifluoromethyl)pyrimidine-5-carboxylic acid (Intermediate 31, 500 mg, 1.80 mmol) and DIPEA (0.63 mL, 3.61 mmol) in DMF (10 mL). The resulting solution was stirred at RT for 15 minutes, 4-aminoadamantan-1-ol hydrochloride (441 mg, 2.16 mmol) was added and stirring continued for 16 h. The reaction mixture was taken up in ethyl acetate, and was washed with water and sat. brine, dried over MgSO₄, filtered and concentrated. The resulting residue was triturated with EtOAc to give the title compound (450 mg, 58%) as a white solid; ¹H NMR (400 MHz) 1.30-1.37 (2H, m), 1.57-1.65 (4H, m), 1.67-1.73 (2H, m), 1.82-1.90 (3H, m), 1.91-2.01 (3H, m), 2.15-2.20 (1H, m), 3.53-3.56 (1H, m), 3.69-3.74 (1H, m), 3.81-3.92 (3H, m), 4.33-4.45 (1H, m), 4.39 (1H, s), 8.23-8.37 (2H, m), 8.50 (1H, s); m/z MH⁺=427; HPLC tR=1.57 min.

### Intermediate 31

### (R)-2-(Tetrahydrofuran-3-ylamino)-4-(trifluoromethyl)pyrimidine-5-carboxylic acid

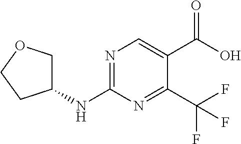

Sodium hydroxide (17.7 mL, 35.3 mmol) was added to (R)-methyl 2-(tetrahydrofuran-3-ylamino)-4-(trifluoromethyl)pyrimidine-5-carboxylate (Intermediate 32, 4.9 g, 16.8 mmol) in MeOH (50 mL) and the resulting solution was stirred at RT for 16 h. The reaction mixture was partially evaporated, diluted with water and acidified to ˜pH3 with 1 M citric acid. The resulting precipitate was filtered, washed with water and dried under high vacuum at 50° C. to give the product (3.67 g, 78%) as a white solid; ¹H NMR (400 MHz) 1.86-1.98 (1H, m), 2.11-2.20 (1H, m), 3.53-3.60 (1H, m), 3.69-3.74 (1H, m), 3.81-3.86 (2H, m), 4.41-4.48 (1H, m), 8.63-8.74 (1H, m), 8.82-8.91 (1H, m); m/z MH⁺=278; HPLC tR=1.42 min.

### Intermediate 32

### (R)-Methyl 2-(tetrahydrofuran-3-ylamino)-4-(trifluoromethyl)pyrimidine-5-carboxylate

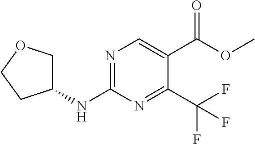

(R)-Tetrahydrofuran-3-amine 4-methylbenzenesulfonate (4.96 g, 19.12 mmol) was added to methyl 2-chloro-4-(trifluoromethyl)pyrimidine-5-carboxylate (commercially available, 4.6 g, 19.1 mmol) and DIPEA (6.66 mL, 38.2 mmol) in THF (40 mL) and the resulting suspension was stirred at RT for 16 h. The reaction mixture was concentrated and taken up in DCM, then was washed sequentially with 1 N citric acid, sat. aq. NaHCO₃ and sat. brine. The organic layer was dried over MgSO₄, filtered and concentrated. The resulting crude mixture was purified by FCC, eluting with 20-50% EtOAc/isohexane to afford the title compound (4.9 g, 88%) as a pale yellow solid; ¹H NMR (400 MHz) 1.91-1.99 (1H, m), 2.16-2.25 (1H, m), 3.59-3.63 (1H, m), 3.71-3.77 (1H, m), 3.83 (3H, s), 3.89-3.93 (2H, m), 4.47-4.51 (1H, m), 8.44 (1H, s), 8.85 (1H, s); m/z MH⁺=292; HPLC tR=1.92 min.

### Example 23

### N-(5-hydroxy-2-adamantyl)-2-[[(3S)-oxolan-3-yl]amino]-4-(trifluoromethyl)pyrimidine-5-carboxamide

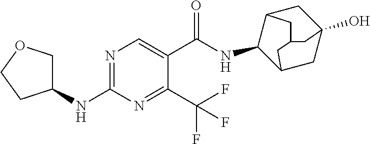

The title compound was prepared in a similar manner to Example 22 from Intermediate 33; ¹H NMR (400 MHz) 1.30-1.37 (2H, m), 1.57-1.65 (4H, m), 1.67-1.73 (2H, m), 1.82-1.90 (3H, m), 1.91-2.01 (3H, m), 2.15-2.20 (1H, m), 3.53-3.56 (1H, m), 3.69-3.74 (1H, m), 3.81-3.92 (3H, m), 4.33-4.45 (1H, m), 4.39 (1H, s), 8.23-8.37 (2H, m), 8.50 (1H, s); m/z MH⁺=427; HPLC tR=1.56 min.

### Intermediate 33

### (S)-2-(Tetrahydrofuran-3-ylamino)-4-(trifluoromethyl)pyrimidine-5-carboxylic acid

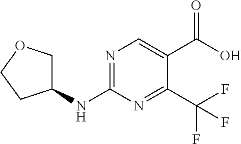

Sodium hydroxide (15.90 mL, 31.73 mmol) was added to (S)-methyl 2-(tetrahydrofuran-3-ylamino)-4-(trifluoromethyl)pyrimidine-5-carboxylate (Intermediate 34, 4.4 g, 15.1 mmol) in MeOH (40 mL) and the resulting solution stirred at RT for 16 h. The reaction was partially concentrated, diluted with water and acidified with 1 M aq. citric acid. The resulting precipitate was filtered, washed with water and dried under high vacuum at 50° C. to afford the title compound (3.3 g, 80%); ¹H NMR (400 MHz) 1.86-1.98 (1H, m), 2.11-2.20 (1H, m), 3.53-3.60 (1H, m), 3.69-3.74 (1H, m), 3.81-3.86 (2H, m), 4.41-4.48 (1H, m), 8.63-8.74 (1H, m), 8.82-8.91 (1H, m); m/z MH⁺=278; HPLC tR=1.41 min.

### Intermediate 34

### (S)-Methyl 2-(tetrahydrofuran-3-ylamino)-4-(trifluoromethyl)pyrimidine-5-carboxylate

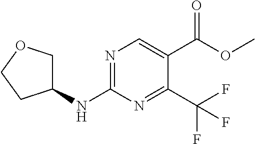

(S)-Tetrahydrofuran-3-amine hydrochloride (2.31 g, 18.7 mmol) was added to methyl 2-chloro-4-(trifluoromethyl)pyrimidine-5-carboxylate (commercially available, 3.0 g, 12.5 mmol) and DIPEA (4.34 mL, 24.9 mmol) in THF (30 mL) and the resulting suspension was stirred at RT for 16 h. The reaction mixture was concentrated, taken up in DCM and washed sequentially with 1 N aq. citric acid, sat. aq. NaHCO₃ and sat. brine. The organic layer was dried over MgSO₄, filtered and concentrated to afford crude product which was purified by FCC, eluting with 20-50% EtOAc/isohexane to afford the title compound (3.3 g, 91%); ¹H NMR (400 MHz) 1.91-1.99 (1H, m), 2.16-2.25 (1H, m), 3.59-3.63 (1H, m), 3.71-3.77 (1H, m), 3.83 (3H, s), 3.89-3.93 (2H, m), 4.47-4.51 (1H, m), 8.44 (1H, s), 8.85 (1H, s); m/z MH⁺=292; HPLC tR=1.91 min.

### Example 24

### AZ13300719 4-Ethoxy-N-(5-hydroxy-2-adamantyl)-2-[[(3R)-tetrahydrofuran-3-yl]amino]pyrimidine-5-carboxamide

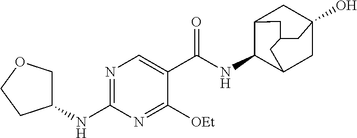

(R)-Tetrahydrofuran-3-amine 4-methylbenzenesulfonate (572 mg, 2.21 mmol), 2-chloro-4-ethoxy-N-(5-hydroxy-2-adamantyl)pyrimidine-5-carboxamide (Intermediate 35, 517 mg, 1.47 mmol) and DIPEA (0.512 mL, 2.94 mmol) were suspended in THF (20 mL) and sealed into a microwave tube. The reaction was heated to 100° C. for 1.5 h in the microwave reactor and cooled to RT. The reaction mixture was concentrated, taken up in DCM, and washed sequentially with sat. aq. NaHCO₃ and water. The organic layer was dried over MgSO₄, filtered and evaporated. The resulting crude product was purified by preparative HPLC to afford the title compound (212 mg, 36%) as a white solid; ¹H NMR (400 MHz) 1.41 (3H, t), 1.47 (2H, d), 1.67 (3H, d), 1.69 (1H, s), 1.75 (2H, d), 1.78 (2H, s), 1.88-1.96 (1H, m), 2.05 (3H, s), 2.14-2.23 (1H, m), 3.58 (1H, dd), 3.70-3.76 (1H, m), 3.84 (1H, dd), 3.92 (1H, dd), 3.99-4.02 (1H, m), 4.41-4.47 (1H, m), 4.53 (2H, q), 7.50 (1H, d), 7.57 (1H, d), 8.61 (1H, s); m/z MH⁺=403; HPLC tR=1.65 min.

### Intermediate 35

### 2-Chloro-4-ethoxy-N-(5-hydroxy-2-adamantyl)pyrimidine-5-carboxamide

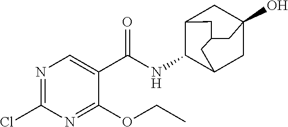

Sodium ethoxide (1.27 mL, 21% in EtOH, 3.41 mmol) was added dropwise to 2,4-dichloro-N-(5-hydroxy-2-adamantyl)pyrimidine-5-carboxamide (Intermediate 36, 1.11 g, 3.24 mmol) in THF (50 mL) at 0° C. The resulting suspension was allowed to warm to RT. The suspension was stirred for a further 4 hours. The reaction mixture was diluted with EtOAc and washed sequentially with 0.1 M aq. HCl, water and sat. brine. The organic layer was dried over MgSO₄, filtered and evaporated to afford the title compound (1.03 g, 91%); ¹H NMR (400 MHz) 1.37 (3H, q), 1.41 (2H, d), 1.63 (3H, s), 1.65 (1H, s), 1.70 (2H,$), 1.73 (1H, s), 1.80 (1H, s), 1.84 (1H,$), 2.04-2.06 (3H, m), 3.93-3.96 (1H, m), 4.47 (2H, q), 7.99 (1H, d), 8.66 (1H, s); m/z MH⁺=352; HPLC t_{R}=1.90 min.

### Intermediate 36

### 2,4-Dichloro-N-(5-hydroxy-2-adamantyl)pyrimidine-5-carboxamide

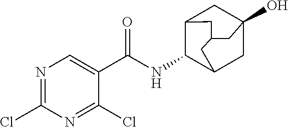

A suspension of 4-aminoadamantan-1-ol hydrochloride (2.89 g, 14.2 mmol) in THF (20 mL) was added dropwise to a stirred solution of 2,4-dichloropyrimidine-5-carbonyl chloride (CAS No. 2972-52-3, 3.00 g, 14.2 mmol) and DIPEA (4.91 mL, 28.4 mmol) in DCM (20 mL) at −10° C. The resulting suspension was stirred at 0° C. for 4 h. The reaction mixture was diluted with DCM and washed sequentially with 0.1 M aq. HCl, water and sat. brine. The organic layer was dried over MgSO₄, filtered and evaporated. The crude solid was triturated with ice-cold DCM to afford the title compound (3.20 g, 66%) as a tan solid; ¹H NMR (400 MHz) 1.36 (2H, d), 1.63 (4H, d), 1.71-1.77 (3H, m), 1.86 (2H, d), 1.98-2.00 (1H, m), 2.06 (2H, s), 3.95 (1H, t), 8.51 (1H, d), 8.83-8.85 (1H, m); m/z MH⁺=342; HPLC t_{R}=1.44 min.

### Example 25

### 4-Ethoxy-N-(5-hydroxy-2-adamantyl)-2-[[(3S)-tetrahydrofuran-3-yl]amino]pyrimidine-5-carboxamide

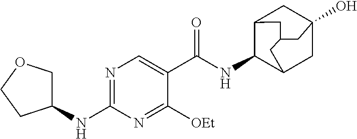

The title compound was prepared in a similar manner to Example 24 from (S)-tetrahydrofuran-3-amine hydrochloride and Intermediate 35; ¹H NMR (400 MHz) 1.42 (5H, m), 1.68 (8H, m), 1.89 (1H, m), 2.02 (3H, m), 2.15 (1H, m), 3.54 (1H, m), 3.70 (1H, q), 3.89 (3H, m), 4.42 (4H, m), 7.67 (1H, d), 7.92 (1H, d), 8.58 (1H, s); m/z MH⁺=403; HPLC tR=1.57 min.

### Example 26

### 4-(Cyclobutoxy)-N-(5-hydroxy-2-adamantyl)-2-[[(3S)-tetrahydrofuran-3-yl]amino]pyrimidine-5-carboxamide

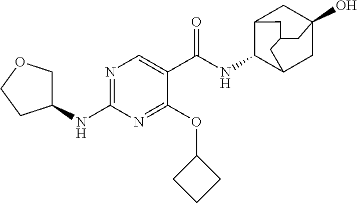

DIPEA (0.347 mL, 1.98 mmol) was added to 2-chloro-4-cyclobutyloxy-N-(5-hydroxy-2-adamantyl)pyrimidine-5-carboxamide (Intermediate 37, 300 mg, 0.79 mmol) and (S)-tetrahydrofuran-3-amine hydrochloride (245 mg, 1.98 mmol) in butyronitrile (3 mL). The reaction mixture was sealed into a microwave tube. The reaction was heated at 150° C. for 40 minutes in the microwave reactor and cooled to RT. The reaction mixture was evaporated to dryness, taken up in DCM and washed with water. The organic layer was dried by passing down an isolute phase separator and evaporated to afford crude product. The crude product was purified by preparative HPLC to afford the title compound (115 mg, 33.7%) as a white solid; ¹H NMR (400 MHz) 1.47 (2H, d), 1.69 (9H, m), 1.86 (2H, m), 2.09 (6H, m), 2.47 (2H, m), 3.55 (1H, m), 3.71 (1H, m), 3.85 (2H, m), 3.98 (1H, m), 4.38 (2H, m), 5.27 (1H, m), 7.65 (1H, d), 7.90 (1H, d), 8.58 (1H, s); m/z MH⁺=429; HPLC tR=1.85 min.

### Intermediate 37

### 2-Chloro-4-cyclobutyloxy-N-(5-hydroxy-2-adamantyl)pyrimidine-5-carboxamide

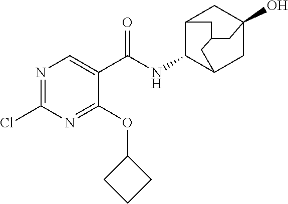

Sodium bis(trimethylsilyl)amide (1.0 M in THF) (3.07 mL, 3.07 mmol) was added in one portion to cyclobutanol (0.240 mL, 3.07 mmol), in THF (5 mL) at RT. The resulting suspension was stirred at RT for 5 minutes. This suspension was added dropwise to 2,4-dichloro-N-(5-hydroxy-2-adamantyl)pyrimidine-5-carboxamide (Intermediate 36, 1 g, 2.92 mmol) in THF (95 mL) at 0°. The resulting suspension was allowed to warm to RT and stirred for a further 4 h. The reaction mixture was diluted with EtOAc and washed sequentially with 0.1 M aq. HCl, water and sat. brine. The organic layer was dried over MgSO₄, filtered and evaporated. The resulting foam was triturated sequentially with EtOAc and isohexane to afford the title compound (0.558 g, 51%) as a yellow solid; ¹H NMR (400 MHz) 1.34 (2H, d), 1.61 (7H, m), 1.76 (3H, m), 1.97 (3H, m), 2.08 (2H, m), 2.35 (2H, m), 3.87 (1H, m), 5.18 (1H, m), 7.92 (1H, d), 8.58 (1H, s); m/z MH⁺=378; HPLC tR=2.13 min.

The following examples were made in a similar manner to Example 26 from the appropriate amine and 2-chloro-4-cyclobutyloxy-N-(5-hydroxy-2-adamantyl)pyrimidine-5-carboxamide (Intermediate 37):

| Structure | Ex no. | name | ¹H NMR | LCMS |
|---|---|---|---|---|
| 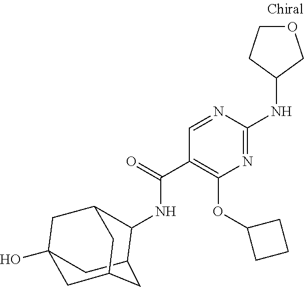 | 27 | 4-(cyclobutoxy)-N- (5-hydroxy-2- adamantyl)-2-[[(3R)- tetrahydrofuran-3- yl]amino]pyrimidine- 5-carboxamide | (400 MHz) 1.47 (2H, d), 1.69 (9H, m), 1.86 (2H, m), 2.08 (6H, m), 2.47 (2H, m), 3.54 (1H, m), 3.71 (1H, m), 3.85 (2H, m), 3.98 (1H, m), 4.39 (2H, m), 5.28 (1H, m), 7.65 (1H, d), 7.89 (1H, d), 8.58 (1H, s) | MH⁺ = 429; HPLC tR = 1.85 min. |
| 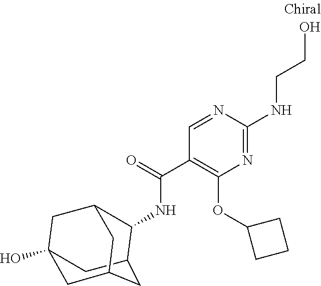 | 28 | 4-(cyclobutoxy)-N- (5-hydroxy-2- adamantyl)-2-(2- hydroxyethylamino) pyrimidine-5- carboxamide | (400 MHz) 1.47 (2H, d), 1.68 (9H, m), 1.83 (1H, q), 2.07 (5H, m), 2.45 (2H, m), 3.34 (2H, m), 3.34 (2H, m), 3.51 (2H, m), 3.97 (1H, m), 4.44 (1H, s), 4.66 (1H, t), 5.25 (1H, m), 7.54 (1H, d), 7.64 (1H, d), 8.56 (1H, m) | MH⁺ = 403; HPLC tR = 1.66 min. |
| 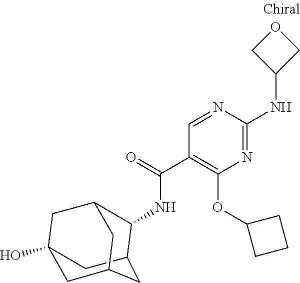 | 29 | 4-(cyclobutoxy)-N- (5-hydroxy-2- adamantyl)-2- (oxetan-3- ylamino)pyrimidine- 5-carboxamide | (400 MHz) 1.53 (2H, d), 1.75 (9H, m), 1.90 (1H, m), 2.14 (5H, m), 2.51 (2H, m), 4.04 (1H, m), 4.50 (1H, s), 4.59 (2H, t), 4.84 (2H, s), 4.97 (1H, s), 5.32 (1H, m), 7.71 (1H, d), 8.44 (1H, d), 8.64 (1H, s) | MH⁺ = 415; HPLC tR = 1.78 min. |
| 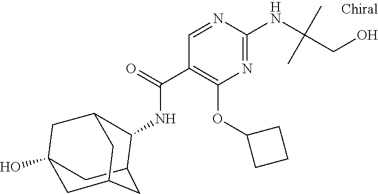 | 30 | 4-(cyclobutoxy)-N- (5-hydroxy-2- adamantyl)-2-[(2- hydroxy-1,1- dimethyl- ethyl)amino] pyrimidine-5- carboxamide | (400 MHz) 1.32 (6H, s), 1.48 (2H, d), 1.69 (9H, m), 1.85 (1H, m), 2.09 (5H, m), 2.47 (2H, m), 3.50 (2H, d), 3.98 (1H, m), 4.43 (1H, s), 5.27 (1H, m), 6.97 (1H, s), 7.64 (1H, d), 8.54 (1H, s) | MH⁺ = 431; HPLC tR = 1.88 min. |
| 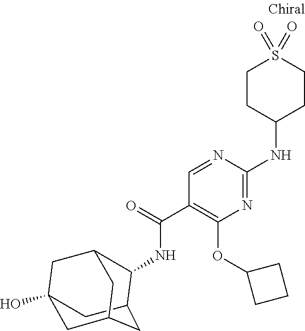 | 31 | 4-(cyclobutoxy)-2- [(1,1-dioxothian-4- yl)amino]-N-(5- hydroxy-2- adamantyl) pyrimidine-5- carboxamide | (400 MHz) 1.54 (2H, d), 1.75 (9H, m), 1.91 (1H, m), 2.15 (9H, m), 2.53 (2H, m), 3.19 (2H, m), 3.35 (2H, m), 4.04 (1H, m), 4.21 (1H, m), 4.50 (1H, s), 5.35 (1H, m), 7.72 (1H, d), 7.90 (1H, d), 8.65 (1H, s) | MH⁺ = 491; HPLC tR = 1.75 min. |
| 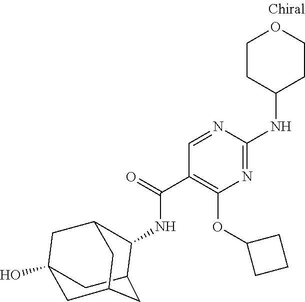 | 32 | 4-(cyclobutoxy)-N- (5-hydroxy-2- adamantyl)-2- (tetrahydropyran-4- ylamino)pyrimidine- 5-carboxamide | 1H NMR (400.132 MHz, DMSO) ä 1.50 (4H, m), 1.74 (12H, m), 2.09 (5H, m), 2.44 (2H, m), 3.37 (2H, m), 3.92 (4H, m), 4.43 (1H, s), 5.25 (1H, t), 7.63 (1H, d), 7.68 (1H, d), 8.57 (1H, s) | m/z MH⁺ = 443; HPLC tR = 1.90 min. |

### Example 33

### N-(5-hydroxy-2-adamantyl)-4-propoxy-2-[[(3R)-tetrahydrofuran-3-yl]amino]pyrimidine-5-carboxamide

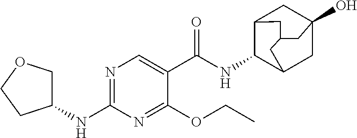

DIPEA (0.492 mL, 2.83 mmol) was added to a suspension of 2-chloro-N-(5-hydroxy-2-adamantyl)-4-propoxypyrimidine-5-carboxamide (Intermediate 38, 517 mg, 1.41 mmol), and (R)-tetrahydrofuran-3-amine 4-methylbenzenesulfonate (550 mg, 2.12 mmol) in butyronitrile (12 mL). The mixture was sealed into a microwave vial. The reaction was heated to 150° C. for 1 h in the microwave reactor and cooled to RT. The reaction mixture was diluted with EtOAc and washed with sat. brine. The organic layer was dried over MgSO₄, filtered and concentrated. The residue was purified by preparative HPLC to afford the title compound (228 mg, 39%); ¹H NMR (400 MHz) 0.98 (3H, t), 1.44 (2H, d), 1.57-1.95 (11H, m), 2.01 (3H, s), 2.14 (1H, s), 3.55 (1H, s), 3.70 (1H, m), 3.76-4.01 (3H, m), 4.43 (4H, m), 7.63 (1H, d), 7.93 (1H, d), 8.59 (1H, s); m/z MH⁺=417; HPLC tR=1.70 min.

### Intermediate 38

### 2-Chloro-N-(5-hydroxy-2-adamantyl)-4-propoxypyrimidine-5-carboxamide

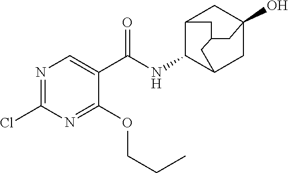

Sodium bis(trimethylsilyl)amide (1 M solution in THF, 1 mL, 1 mmol) was added in one portion to 1-propanol (0.075 mL, 1 mmol), in THF (1 mL) at RT. The resulting suspension was stirred at RT. This suspension was added dropwise to 2,4-dichloro-N-(5-hydroxy-2-adamantyl)pyrimidine-5-carboxamide (Intermediate 36, 0.342 g, 1 mmol) in THF (10 mL) at RT under nitrogen. The resulting suspension was stirred for a further 4 h. The reaction mixture was diluted with EtOAc and washed sequentially with 0.1 M aq. HCl, water and sat. brine. The organic layer was dried over MgSO₄, filtered and evaporated to afford the title compound as a crude product that was used without further purification; m/z MH⁺=366; HPLC t_{R}=2.03 min.

### Example 34

### N-(5-hydroxy-2-adamantyl)-4-propoxy-2-[[(3S)-tetrahydrofuran-3-yl]amino]pyrimidine-5-carboxamide

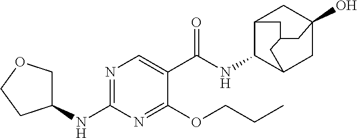

The title compound was prepared in a similar manner to Example 33 from 2-Chloro-N-(5-hydroxy-2-adamantyl)-4-propoxypyrimidine-5-carboxamide (Intermediate 38) and (S)-tetrahydrofuran-3-amine hydrochloride; ¹H NMR (400 MHz) 0.98 (3H, t), 1.44 (2H, d), 1.59-1.95 (11H, m), 2.01 (3H, s), 2.14 (1H, s), 3.55 (1H, s), 3.70 (1H, dd), 3.78-4.00 (3H, m), 4.42 (4H, d), 7.63 (1H, d), 7.93 (1H, d), 8.59 (1H, s); m/z MH⁺=417; HPLC tR=1.75 min.

### Example 35

### N-(5-hydroxy-2-adamantyl)-4-isopropoxy-2-[[(3S)-tetrahydrofuran-3-yl]amino]pyrimidine-5-carboxamide

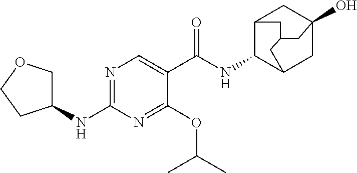

2-Chloro-N-(5-hydroxy-2-adamantyl)-4-propan-2-yloxypyrimidine-5-carboxamide (Intermediate 39, 560 mg, 1.53 mmol), and (S)-tetrahydrofuran-3-amine hydrochloride (284 mg, 2.30 mmol) were suspended in butyronitrile (12 mL). DIPEA (0.533 mL, 3.06 mmol) was added and the mixture was sealed into a microwave vial. The reaction was heated to 150° C. for 2.5 h in the microwave reactor, then was cooled to RT. The reaction mixture was diluted with EtOAc and washed with sat. brine. The organic layer was dried over MgSO₄ and concentrated. The crude product was purified by preparative HPLC to afford the title compound (149 mg, 23%); ¹H NMR (400 MHz) 1.38 (6H, d), 1.47 (2H, d), 1.68 (8H, m), 1.90 (1H, m), 2.02 (3H, d), 2.13 (1H, m), 3.55 (1H, s), 3.70 (1H, dd), 3.77-3.94 (2H, m), 3.95-4.01 (1H, m), 4.39 (2H, m), 5.50 (1H, hept), 7.66 (1H, d), 7.92 (1H, m), 8.60 (1H, s); m/z MH⁺=417; HPLC tR=1.58 min.

### Intermediate 39

### 2-Chloro-N-(5-hydroxy-2-adamantyl)-4-propan-2-yloxypyrimidine-5-carboxamide

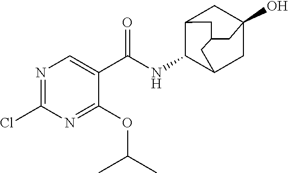

The title compound was prepared in a similar manner to Intermediate 38 from 2,4-dichloro-N-(5-hydroxy-2-adamantyl)pyrimidine-5-carboxamide (Intermediate 36) and isopropanol; m/z MH⁺=366; HPLC t_{R}=2.01 min.

### Example 36

### N-(5-hydroxy-2-adamantyl)-4-isopropoxy-2-[[(3R)-tetrahydro furan-3-yl]amino]pyrimidine-5-carboxamide

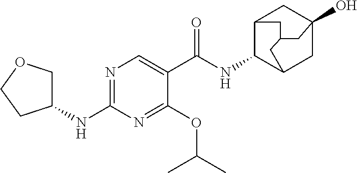

The title compound was prepared in a similar manner to Example 35 from 2-Chloro-N-(5-hydroxy-2-adamantyl)-4-propan-2-yloxypyrimidine-5-carboxamide (Intermediate 39) and (R)-tetrahydrofuran-3-amine 4-methylbenzenesulfonate; ¹H NMR (400 MHz, DMSO) δ 1.38 (6H, d), 1.47 (2H, d), 1.68 (8H, m), 1.90 (1H, m), 2.02 (3H, d), 2.13 (1H, m), 3.55 (1H, s), 3.70 (1H, dd), 3.77-3.94 (2H, m), 3.95-4.02 (1H, m), 4.39 (2H, m), 5.50 (1H, hept), 7.66 (1H, d), 7.92 (1H, m), 8.60 (1H, s); m/z MH⁺=417; HPLC tR=1.58 min.

### Example 37

### N-(5-hydroxy-2-adamantyl)-4-pyrrolidin-1-yl-2-[[(3R)-tetrahydrofuran-3-yl]amino]pyrimidine-5-carboxamide

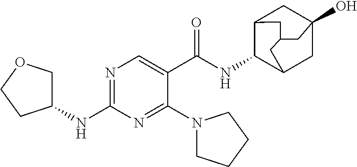

DIPEA (0.564 mL, 3.24 mmol) was added to 2-chloro-N-(5-hydroxy-2-adamantyl)-4-pyrrolidin-1-ylpyrimidine-5-carboxamide (Intermediate 40, 610 mg, 1.62 mmol) and (R)-tetrahydrofuran-3-amine 4-methylbenzenesulfonate (588 mg, 2.27 mmol) in butyronitrile (12 mL). The reaction mixture was heated to 150° C. for 2.5 h in a microwave reactor and then allowed to cooled to RT. The reaction mixture was diluted with EtOAc and washed with sat. brine. The organic layer was dried over MgSO₄, filtered and concentrated. The residue was purified by preparative HPLC, then repurified by recrystallisation from EtOAc to afford the title compound (284 mg, 41%); ¹H NMR (400 MHz) 1.30 (2H, d), 1.60 (4H, m), 1.69 (2H, m), 1.77-2.04 (10H, m), 2.05-2.15 (1H, m), 3.36 (4H, m), 3.48 (1H, dd), 3.68 (1H, td), 3.75-3.92 (3H, m), 4.27-4.38 (2H, m), 6.90 (1H, s), 7.78 (1H, s), 7.95 (1H, d); m/z MH⁺=428; HPLC tR=1.49 min.

### Intermediate 40

### 2-Chloro-N-(5-hydroxy-2-adamantyl)-4-pyrrolidin-1-ylpyrimidine-5-carboxamide

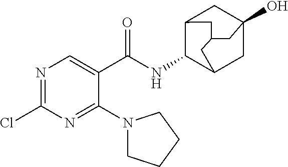

DIPEA (0.570 mL, 3.27 mmol) was added to 2,4-dichloro-N-(5-hydroxy-2-adamantyl)pyrimidine-5-carboxamide (Intermediate 36, 1.02 g, 2.97 mmol), and pyrrolidine (0.248 mL, 2.97 mmol) in butyronitrile (12 mL). This mixture was stirred at RT for 16 hours, then was diluted with EtOAc, washed with sat. brine, dried over MgSO₄ and concentrated. The residue was purified by FCC, eluting with 0-10% MeOH/DCM, to afford the title compound (0.61 g, 54%); ¹H NMR (400 MHz) 1.33 (2H, d), 1.62 (4H, m), 1.70 (2H, m), 1.80-1.94 (6H, m), 2.00 (3H, m), 3.41 (4H, s), 3.89 (1H, d), 4.41 (1H, s), 7.96 (1H, s), 8.37 (1H, d); m/z MH⁺=377; HPLC tR=1.49 min.

### Example 38

### N-(5-hydroxy-2-adamantyl)-4-pyrrolidin-1-yl-2-[[(3S)-tetrahydrofuran-3-yl]amino]pyrimidine-5-carboxamide

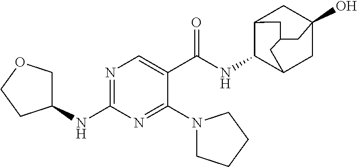

The title compound was prepared in a similar manner to Example 37 from 2-chloro-N-(5-hydroxy-2-adamantyl)-4-pyrrolidin-1-ylpyrimidine-5-carboxamide (Intermediate 40) and (S)-tetrahydrofuran-3-amine hydrochloride; ¹H NMR (400 MHz) 1.30 (2H, d), 1.60 (4H, d), 1.69 (2H, d), 1.76-2.04 (10H, m), 2.04-2.16 (1H, m), 3.36 (4H, d), 3.48 (1H, dd), 3.68 (1H, td), 3.75-3.92 (3H, m), 4.26-4.39 (2H, m), 6.90 (1H, s), 7.78 (1H, s), 7.95 (1H, d); m/z MH⁺=428; HPLC tR=1.35 min.

### Example 39

### N-(5-hydroxy-2-adamantyl)-4-pyrazol-1-yl-2-[[(3R)-tetrahydrofuran-3-yl]amino]pyrimidine-5-carboxamide

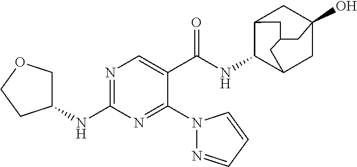

DIPEA (0.416 mL, 2.39 mmol) was added to 2-chloro-N-(5-hydroxy-2-adamantyl)-4-pyrazol-1-ylpyrimidine-5-carboxamide (Intermediate 41, 406 mg, 1.09 mmol), and (R)-tetrahydrofuran-3-amine 4-methylbenzenesulfonate (563 mg, 2.17 mmol) in butyronitrile (12 mL). The reaction was heated at 150° C. for 2 h in a microwave reactor and cooled to RT. The reaction mixture was diluted with EtOAc and washed with sat. brine. The organic layer was dried over MgSO₄, filtered and concentrated. This was purified by preparative to afford the title compound (88 mg, 19%); ¹H NMR (400 MHz) 1.24 (2H, d), 1.59 (3H, d), 1.69 (2H, d), 1.76 (2H, d), 1.85-1.96 (2H, m), 2.00 (2H, s), 2.13-2.25 (1H, m), 3.57 (1H, dd), 3.73 (1H, td), 3.80-4.00 (3H, m), 4.35 (1H, s), 4.41-4.50 (1H, m), 6.52 (1H, dd), 7.70 (1H, d), 7.82-7.98 (2H, m), 8.27 (1H, s), 8.48 (1H, s); m/z MH⁺=425; HPLC tR=1.26 min.

### Intermediate 41

### 2-Chloro-N-(5-hydroxy-2-adamantyl)-4-pyrazol-1-ylpyrimidine-5-carboxamide

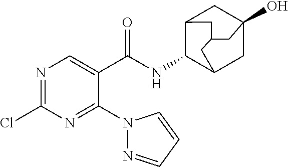

Sodium bis(trimethylsilyl)amide (4.60 mL, 4.60 mmol) was added in one portion to 1H-pyrazole (0.298 g, 4.38 mmol), in THF (10 mL) at RT. The resulting suspension was stirred at RT for 10 minutes. This suspension was added dropwise to 2,4-dichloro-N-(5-hydroxy-2-adamantyl)pyrimidine-5-carboxamide (Intermediate 36, 1.5 g, 4.38 mmol) in THF (15 mL) at 0°. The resulting suspension was stirred for 5 h. The reaction mixture was diluted with EtOAc and washed with sat. brine. The organic layer was dried over MgSO₄, filtered and concentrated. This was purified by FCC, eluting with 0-10% MeOH/DCM, to afford the title compound (0.801 g, 49%); ¹H NMR (400 MHz) 1.28 (2H, d), 1.55-1.67 (3H, m), 1.67-1.78 (5H, m), 1.91 (1H, s), 2.06 (2H, s), 3.95 (1H, d), 4.39 (1H, s), 6.64 (1H, dd), 7.84 (1H, d), 8.17 (1H, d), 8.58-8.62 (1H, m), 8.71 (1H, s); m/z MH⁺=374; HPLC tR=1.52 min.

### Example 40

### N-(5-hydroxy-2-adamantyl)-4-pyrazol-1-yl-2-[[(3S)-tetrahydro furan-3-yl]amino]pyrimidine-5-carboxamide

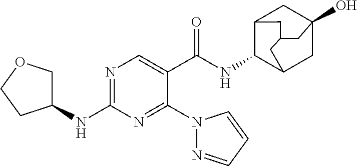

The title compound was prepared in a similar manner to Example 39 from 2-chloro-N-(5-hydroxy-2-adamantyl)-4-pyrazol-1-ylpyrimidine-5-carboxamide (Intermediate 41) and (S)-tetrahydrofuran-3-amine hydrochloride; ¹H NMR (400 MHz) 1.24 (2H, d), 1.59 (3H, d), 1.69 (2H, d), 1.76 (2H, d), 1.85-1.96 (2H, m), 2.00 (2H, s), 2.12-2.25 (1H, m), 3.57 (1H, dd), 3.73 (1H, td), 3.80-4.00 (3H, m), 4.35 (1H, s), 4.41-4.50 (1H, m), 6.52 (1H, dd), 7.70 (1H, dd), 7.80-7.98 (2H, m), 8.27 (1H, s), 8.48 (1H, s); m/z MH⁺=425; HPLC tR=1.29 min.

### Example 41

### 4-Cyclopentyl-N-(5-hydroxy-2-adamantyl)-N-methyl-2-[[(3R)-oxolan-3-yl]amino]pyrimidine-5-carboxamide

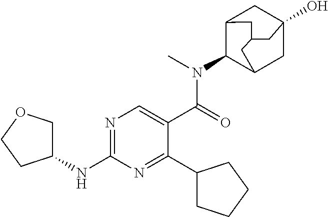

DIPEA (1.108 mL, 6.34 mmol) was added to 4-cyclopentyl-N-(5-hydroxy-2-adamantyl)-N-methyl-2-methylsulfonylpyrimidine-5-carboxamide (Intermediate 42, 1.1 g, 2.54 mmol) and (R)-tetrahydrofuran-3-amine 4-methylbenzenesulfonate (0.658 g, 2.54 mmol) in butyronitrile (10 mL). The reaction mixture was heated at 150° C. for 1 h in a microwave reactor and then cooled to RT. The reaction mixture was evaporated to dryness, taken up in DCM, washed with water and then dried by passing the organic layer through an isolute phase separator and concentrated. The crude product was purified by preparative HPLC and then repurified by FCC, eluting with 0-10% MeOH/EtOAc to afford the title compound (250 mg, 22%); ¹H NMR (400 MHz) 1.43-1.50 (2H, m), 1.54-1.94 (17H, m), 2.08-2.17 (2H, m), 2.30-2.33 (2H, m), 2.99-3.06 (1H, m), 3.01 (3H, s), 3.52-3.55 (1H, m), 3.67-3.73 (1H, m), 3.80-3.88 (2H, m), 4.00-4.05 (1H, m), 4.32-4.36 (1H, m), 4.41 (1H, s), 7.43-7.49 (1H, m), 8.04 (1H, s); m/z MH⁺=441; HPLC tR=1.93 min.

### Intermediate 42

### 4-Cyclopentyl-N-(5-hydroxy-2-adamantyl)-N-methyl-2-methylsulfonylpyrimidine-5-carboxamide

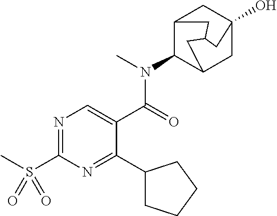

Oxone (8.88 g, 14.4 mmol) was added portionwise to 4-cyclopentyl-N-(5-hydroxy-2-adamantyl)-N-methyl-2-methylsulfanylpyrimidine-5-carboxamide (Intermediate 43, 2.9 g, 7.22 mmol) in acetonitrile and water and the resulting suspension was stirred at RT for 16 h. The reaction mixture was concentrated. Sat. aq. NaHCO₃ was added and the mixture was extracted with DCM. The organic layer was dried over MgSO₄, filtered and evaporated to afford the title compound (3.0 g, 95%) as a white solid; m/z MH⁺=434; HPLC tR=1.82 min.

### Intermediate 43

### 4-Cyclopentyl-N-(5-hydroxy-2-adamantyl)-N-methyl-2-methylsulfanylpyrimidine-5-carboxamide

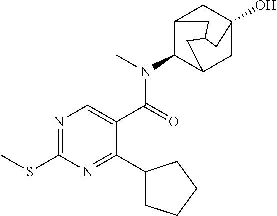

Sodium hydride (0.103 g, 2.58 mmol) was added portionwise to 4-cyclopentyl-N-(5-hydroxy-2-adamantyl)-2-methylsulfanylpyrimidine-5-carboxamide (Intermediate 9, 1.0 g, 2.58 mmol) in DMF (5 mL) over a period of 2 minutes and the resulting suspension was stirred at RT for 30 minutes. The reaction mixture was cooled to 5° C., iodomethane (0.161 mL, 2.58 mmol) in DMF (1 mL) was added over a period of 1 minute and the reaction was stirred at RT for 16 h. The reaction mixture was poured into 1 M aq. citric acid and extracted with EtOAc. The organic layer was washed with water and sat. brine, dried over MgSO₄, filtered and evaporated to afford the title compound; m/z MH⁺=402; HPLC tR=2.3 min.

### Example 42

### 4-cyclopentyl-N-(5-hydroxy-2-adamantyl)-N-methyl-2-[[(3S)-oxolan-3-yl]amino]pyrimidine-5-carboxamide

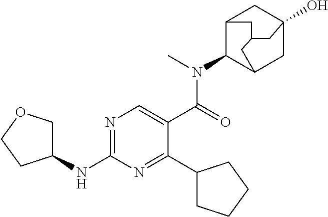

The title compound was prepared in a similar manner to Example 42 from 4-cyclopentyl-N-(5-hydroxy-2-adamantyl)-N-methyl-2-methylsulfonylpyrimidine-5-carboxamide (Intermediate 42); ¹H NMR (400 MHz) 1.43-1.50 (2H, m), 1.54-1.94 (17H, m), 2.08-2.17 (2H, m), 2.30-2.33 (2H, m), 2.99-3.06 (1H, m), 3.01 (3H, s), 3.52-3.55 (1H, m), 3.67-3.73 (1H, m), 3.80-3.88 (2H, m), 4.00-4.05 (1H, m), 4.32-4.36 (1H, m), 4.41 (1H, s), 7.43-7.49 (1H, m), 8.04 (1H, s); m/z MH⁺=441; HPLC tR=1.89 min.

### Example 43

### N-(5-carbamoyl-2-adamantyl)-4-cyclopentyl-2-[[(3S)-tetrahydrofuran-3-yl]amino]pyrimidine-5-carboxamide

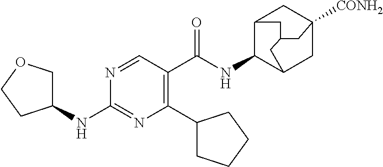

DIPEA (0.231 ml, 1.32 mmol) was added to 4-[[4-cyclopentyl-2-[[(3S)-oxolan-3-yl]amino]pyrimidine-5-carbonyl]amino]adamantane-1-carboxylic acid (Example 45, 200 mg, 0.44 mmol) and HATU (335 mg, 0.88 mmol) in DMF (5 mL). The resulting solution was stirred at RT for 20 minutes. Ammonium chloride (47 mg, 0.88 mmol) was added and the reaction mixture was stirred at RT for 1 h. The reaction mixture was quenched with sat. aq. NaHCO₃. The resultant precipitate was collected by filtration and was washed with water and dried under vacuum. The resultant solid was triturated with EtOAc to afford the desired product (108 mg, 54%) as a white solid; ¹H NMR (400 MHz) 1.41 (3H, d), 1.56 (4H, m), 1.80 (25H, m), 1.99 (8H, m), 2.12 (2H, m), 3.43 (3H, m), 3.51 (2H, m), 3.70 (2H, q), 3.88 (4H, t), 3.93 (3H, m), 4.35 (2H, m), 6.83 (3H, d), 7.49 (1H, s), 8.09 (2H, d), 8.15 (2H, s); m/z MH⁺=454; HPLC tR=1.75 min.

### Example 44

### 4-[[4-cyclopentyl-2-[[(3S)-tetrahydrofuran-3-yl]amino]pyrimidine-5-carbonyl]amino]adamantane-1-carboxylic acid

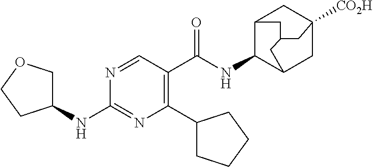

2 M aq. sodium hydroxide (1.81 mL, 3.63 mmol) was added to methyl 4-[[4-cyclopentyl-2-[[(3S)-oxolan-3-yl]amino]pyrimidine-5-carbonyl]amino]adamantane-1-carboxylate (Intermediate 44, 850 mg, 1.81 mmol) in methanol (40 mL) at RT. The resulting mixture was stirred at RT for 16 h, then was stirred at 55° C. for 6 h. The reaction mixture was concentrated, taken up in water and washed with EtOAc. The aqueous layer was acidified with 1 M citric acid and extracted with EtOAc. The organic layer was concentrated, taken up in DCM and dried by passing down an isolute phase separator and evaporated to afford the title compound (613 mg, 74%). A sample of crude material (150 mg) was purified by preparative HPLC to afford the title compound (46 mg, 6%) as a white solid; ¹H NMR (400 MHz) 1.43 (2H, d), 1.57 (2H, m), 1.80 (14H, m), 2.00 (4H, m), 2.13 (1H, m), 3.44 (1H, t), 3.51 (1H, m), 3.70 (1H, m), 3.87 (3H, m), 4.35 (1H, m), 7.49 (1H, s), 8.11 (1H, d), 8.16 (1H, s), 11.96 (1H, s); m/z MH⁺=455; HPLC tR=1.95 min.

### Intermediate 44

### Methyl 4-[[4-cyclopentyl-2-[[(3S)-tetrahydrofuran-3-yl]amino]pyrimidine-5-carbonyl]amino]adamantane-1-carboxylate

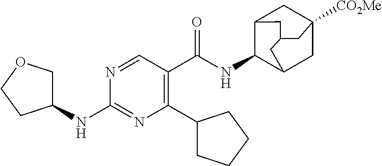

DIPEA (2.02 ml, 11.5 mmol) was added to (S)-4-cyclopentyl-2-(tetrahydrofuran-3-ylamino)pyrimidine-5-carboxylic acid (Intermediate 45, 800 mg, 2.88 mmol) and HATU (1.65 g, 4.33 mmol) in DMF (5 mL), and the resulting solution was stirred at RT for 20 minutes. Methyl 4-aminoadamantane-1-carboxylate hydrochloride (prepared according to Bioorganic & Medicinal Chemistry Letters 16 (2006) 5958-5962, 1.06 g, 4.33 mmol) was added and the reaction mixture was stirred at RT for 16 h. Additional HATU (1.65 g, 4.33 mmol) and DIPEA (0.189 ml) was added and the reaction mixture was stirred at RT for a further 16 h. The reaction mixture was quenched with sat. aq. NaHCO₃, and the resultant precipitate was washed with water and dried under vacuum to afford the title compound (1.34 g, 99%). A sample of crude material (130 mg) was purified by preparative HPLC to afford the title compound (67 mg, 5%) as a white solid; ¹H NMR (400 MHz) 1.44 (2H, d), 1.56 (2H, m), 1.80 (14H, m), 2.01 (4H, m), 2.13 (1H, m), 3.43 (1H, m), 3.51 (1H, m), 3.59 (3H, s), 3.70 (1H, q), 3.81 (1H, q), 3.90 (2H, m), 4.34 (1H, m), 7.50 (1H, s), 8.11 (1H, d), 8.16 (1H, s); m/z MH⁺=469; HPLC tR=2.45 min.

### Intermediate 45

### (S)-4-Cyclopentyl-2-(tetrahydrofuran-3-ylamino)pyrimidine-5-carboxylic acid

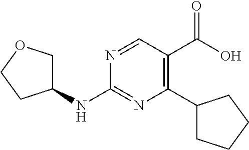

2 M aq. NaOH (8.72 mL, 17.4 mmol) was added to a solution of (S)-methyl 4-cyclopentyl-2-(tetrahydrofuran-3-ylamino)pyrimidine-5-carboxylate (Intermediate 46, 2.54 g, 8.72 mmol) in methanol (40 mL) at RT. The resulting mixture was stirred at 40° C. for 16 h, then the reaction was stirred at 55° C. for 4 h. The reaction mixture was concentrated, taken up in water and washed with EtOAc. The aqueous layer was acidified with 1 M aq. citric acid and the resulting solid was washed with cold water, dried under vacuum to afford the title compound (2.30 g, 95%) as a white solid; ¹H NMR (400 MHz) 1.60 (2H, m), 1.75 (4H, m), 1.88 (3H, m), 2.14 (1H, m), 3.54 (1H, m), 3.71 (1H, q), 3.85 (2H, m), 4.06 (1H, m), 4.40 (1H, m), 7.92 (1H, d), 8.64 (1H, s), 12.42 (1H, s); m/z MH⁺=278; HPLC tR=1.75 min.

### Intermediate 46

### (S)-Methyl 4-cyclopentyl-2-(tetrahydrofuran-3-ylamino)pyrimidine-5-carboxylate

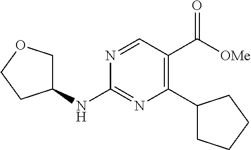

DIPEA (4.91 mL, 28.1 mmol) was added to methyl 4-cyclopentyl-2-(methylsulfonyl)pyrimidine-5-carboxylate (Intermediate 47, 4 g, 14.1 mmol) and (S)-tetrahydrofuran-3-amine hydrochloride (2.61 g, 21.1 mmol) in THF (50 mL). The resulting solution was stirred at RT for 16 h. The reaction mixture was concentrated, taken up in DCM, and washed sequentially with water and sat. aq. NaHCO₃. The organic layer was dried over MgSO₄, filtered and evaporated to afford the title compound (2.55 g, 62%); ¹H NMR (400 MHz) 1.67 (2H, m), 1.82 (4H, m), 1.97 (3H, m), 2.20 (1H, m), 3.61 (1H, m), 3.77 (1H, q), 3.82 (3H, s), 3.97 (3H, m), 4.46 (1H, m), 8.08 (1H, d), 8.72 (1H, s); m/z MH⁺=292; HPLC tR=2.30 min.

### Intermediate 47

### Methyl 4-cyclopentyl-2-(methylsulfonyl)pyrimidine-5-carboxylate

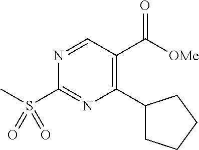

Oxone (48.7 g, 79.3 mmol) was added portionwise to methyl 4-cyclopentyl-2-(methylthio)pyrimidine-5-carboxylate (Intermediate 11, 10 g, 39.6 mmol) in acetonitrile (75 mL) and water (75 mL). The resulting suspension was stirred at RT for 16 h. The reaction mixture was concentrated, sat. aq. NaHCO₃ was added (carefully) and the mixture was extracted with DCM. The organic layers were combined, dried over MgSO₄, filtered and concentrated to afford the title compound (9.70 g, 86%); ¹H NMR (400 MHz) 1.67 (2H, m), 1.81 (4H, m), 2.02 (2H, m), 3.43 (3H, s), 3.92 (1H, m), 3.94 (3H, s), 9.24 (1H, s); m/z MH⁺=285; HPLC tR=2.08 min.

### Example 45

### 4-cyclopentyl-N-(5-methylsulfonyl-2-adamantyl)-2-[[(3S)-tetrahydrofuran-3-yl]amino]pyrimidine-5-carboxamide

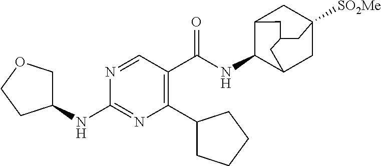

DIPEA (0.567 ml, 3.25 mmol) was added to (S)-4-cyclopentyl-2-(tetrahydrofuran-3-ylamino)pyrimidine-5-carboxylic acid (Intermediate 45, 300 mg, 1.08 mmol) and HATU (494 mg, 1.30 mmol) in DMF (5 mL). The resulting solution was stirred at RT for 20 minutes. 5-methylsulfonyladamantan-2-amine (prepared according to Bioorganic & Medicinal Chemistry Letters 17 (2) (2007) 527-532, 248 mg, 1.08 mmol) was added and the reaction mixture was stirred at RT for 16 h. Additional HATU (494 mg, 1.30 mmol) and DIPEA (0.189 ml) was added and the solution was stirred at RT for a further 4 h. The reaction mixture was quenched with sat. aq. NaHCO₃, and the resultant precipitate was collected by filtration and dried under vacuum. The crude product was purified by preparative HPLC to afford the title compound (177 mg, 34%); ¹H NMR (400 MHz) 1.45 (2H, d), 1.56 (2H, m), 1.80 (9H, m), 2.02 (7H, m), 2.14 (3H, m), 2.84 (3H, s), 3.44 (1H, m), 3.52 (1H, m), 3.71 (1H, m), 3.82 (1H, q), 3.88 (1H, t), 3.96 (1H, m), 4.36 (1H, m), 7.53 (1H, s), 8.17 (2H, m); m/z MH⁺=489; HPLC tR=1.98 min.

1. A compound of formula (I) or a pharmaceutically-acceptable salt thereof: 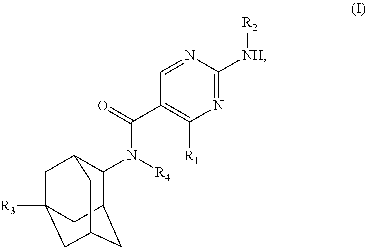
wherein:
R₁ is trifluoromethyl, propyl, cyclobutyl, cyclopentyl, ethoxy, propoxy, cyclobutoxy, pyrrolidinyl, or pyrazolyl;
R₂ is tetrahydrofuranyl, 1-acetylpiperidyl, oxopiperidyl, dioxothiolanyl, hydroxycyclopentyl, hydroxyethyl, hydroxypropyl, 2-(dimethylamino)-2-oxo-ethyl, methoxyethyl, oxopyrrolidinyl, 1-acetylpyrrolidinyl, oxetanyl, hydroxydimethyl-ethyl, 1,1-dioxothianyl, tetrahydropyranyl, (tetrahydrofuranyl)methylenyl, (oxopyrrolidinyl)methylenyl, (methyl-oxo-pyrrolidinyl)methylenyl or pyrrolidinyl;
R₃ is hydroxy, carboxy, carbamoyl, or methylsulphonyl;
R₄ is hydrogen or methyl;
with the proviso that said compound is not:
4-cyclobutyl-N-[(2r,5s)-5-hydroxyadamantan-2-yl]-2-[(2-hydroxy-1,1-dimethylethyl)amino]pyrimidine-5-carboxamide;
4-cyclobutyl-N-[(2r,5s)-5-hydroxyadamantan-2-yl]-2-[(2-hydroxyethyl)amino]pyrimidine-5-carboxamide;
4-cyclobutyl-N-[(2r,5s)-5-hydroxyadamantan-2-yl]-2-[(2-hydroxy-2-methylpropyl)amino]pyrimidine-5-carboxamide;
4-cyclobutyl-N-[(2r,5s)-5-hydroxyadamantan-2-yl]-2-(oxetan-3-ylamino)pyrimidine-5-carboxamide;
4-cyclobutyl-2-[(1,1-dioxidotetrahydro-2H-thiopyran-4-yl)amino]-N-[(2r,5s)-5-hydroxyadamantan-2-yl]pyrimidine-5-carboxamide;
4-cyclopentyl-N-[(2r,5s)-5-hydroxyadamantan-2-yl]-2-oxetan-3-ylamino)pyrimidine-5-carboxamide;
4-cyclopentyl-2-[(1,1-dioxothian-4-yl)amino]-N-[(2r,5s)-5-hydroxyadamantan-2-yl]pyrimidine-5-carboxamide;
4-cyclopentyl-N-[(2r,5s)-5-hydroxyadamantan-2-yl]-2-[(2-hydroxy-2-methylpropyl)amino]pyrimidine-5-carboxamide;
4-cyclopentyl-N-[(2r,5s)-5-hydroxyadamantan-2-yl]-2-(2-hydroxyethylamino)pyrimidine-5-carboxamide;
4-cyclopentyl-N-[(2r,5s)-5-hydroxyadamantan-2-yl]-2-[(1-hydroxy-2-methylpropan-2-yl)amino]pyrimidine-5-carboxamide;
4-cyclopentyl-N-[(2r,5s)-5-hydroxyadamantan-2-yl]-2-(oxan-4-ylamino)pyrimidine-5-carboxamide;
4-cyclopentyl-N-[(2r,5s)-5-hydroxyadamantan-2-yl]-2-[[(3R)-oxolan-3-yl]amino]pyrimidine-5-carboxamide;
4-cyclopentyl-N-[(2r,5s)-5-hydroxyadamantan-2-yl]-2-[[(3S)-oxolan-3-yl]amino]pyrimidine-5-carboxamide;
4-ethoxy-N-[(2r,5s)-5-hydroxytricyclo[3.3.1.1^{3,7}]dec-2-yl]-2-(oxetan-3-ylamino)pyrimidine-5-carboxamide; or
N-[(2r,5s)-5-hydroxytricyclo[3.3.1.1^{3,7}]dec-2-yl]-4-(1-methylethoxy)-2-(oxetan-3-ylamino)pyrimidine-5-carboxamide.

2. A compound according to claim 1, wherein:
R₁ is trifluoromethyl, propyl, cyclobutyl, cyclopentyl, ethoxy, propoxy, or cyclobutoxy;
R₃ is hydroxy or carbamoyl; and
with the proviso that said compound is not:
4-cyclobutyl-N-[(2r,5s)-5-hydroxyadamantan-2-yl]-2-[(2-hydroxy-1,1-dimethylethyl)amino]pyrimidine-5-carboxamide;
4-cyclobutyl-N-[(2r,5s)-5-hydroxyadamantan-2-yl]-2-[(2-hydroxyethyl)amino]pyrimidine-5-carboxamide;
4-cyclobutyl-N-[(2r,5s)-5-hydroxyadamantan-2-yl]-2-[(2-hydroxy-2-methylpropyl)amino]pyrimidine-5-carboxamide;
4-cyclobutyl-N-[(2r,5s)-5-hydroxyadamantan-2-yl]-2-(oxetan-3-ylamino)pyrimidine-5-carboxamide;
4-cyclobutyl-2-[(1,1-dioxidotetrahydro-2H-thiopyran-4-yl)amino]-N-[(2r,5s)-5-hydroxyadamantan-2-yl]pyrimidine-5-carboxamide;
4-cyclopentyl-N-[(2r,5s)-5-hydroxyadamantan-2-yl]-2-oxetan-3-ylamino)pyrimidine-5-carboxamide;
4-cyclopentyl-2-[(1,1-dioxothian-4-yl)amino]-N-[(2r,5s)-5-hydroxyadamantan-2-yl]pyrimidine-5-carboxamide;
4-cyclopentyl-N-[(2r,5s)-5-hydroxyadamantan-2-yl]-2-[(2-hydroxy-2-methylpropyl)amino]pyrimidine-5-carboxamide;
4-cyclopentyl-N-[(2r,5s)-5-hydroxyadamantan-2-yl]-2-(2-hydroxyethylamino)pyrimidine-5-carboxamide;
4-cyclopentyl-N-[(2r,5s)-5-hydroxyadamantan-2-yl]-2-[(1-hydroxy-2-methylpropan-2-yl)amino]pyrimidine-5-carboxamide;
4-cyclopentyl-N-[(2r,5s)-5-hydroxyadamantan-2-yl]-2-(oxan-4-ylamino)pyrimidine-5-carboxamide;
4-cyclopentyl-N-[(2r,5s)-5-hydroxyadamantan-2-yl]-2-[[(3R)-oxolan-3-yl]amino]pyrimidine-5-carboxamide;
4-cyclopentyl-N-[(2r,5s)-5-hydroxyadamantan-2-yl]-2-[[(3S)-oxolan-3-yl]amino]pyrimidine-5-carboxamide;
4-ethoxy-N-[(2r,5s)-5-hydroxytricyclo[3.3.1.1^{3,7}]dec-2-yl]-2-(oxetan-3-ylamino)pyrimidine-5-carboxamide; or
N-[(2r,5s)-5-hydroxytricyclo[3.3.1.1^{3,7}]dec-2-yl]-4-(1-methylethoxy)-2-(oxetan-3-ylamino)pyrimidine-5-carboxamide.

3. One or more compound(s) according to claim 1, selected from the group consisting of:
4-cyclobutyl-N-(5-hydroxy-2-adamantyl)-2-[[(3S)-tetrahydrofuran-3-yl]amino]pyrimidine-5-carboxamide,
4-cyclobutyl-N-(5-hydroxy-2-adamantyl)-2-[[(3R)-tetrahydrofuran-3-yl]amino]pyrimidine-5-carboxamide,
2-[(1-acetyl-4-piperidyl)amino]-4-cyclopentyl-N-(5-hydroxy-2-adamantyl)pyrimidine-5-carboxamide,
4-cyclopentyl-N-(5-hydroxy-2-adamantyl)-2-[[(3R)-6-oxo-3-piperidyl]amino]pyrimidine-5-carboxamide,
4-cyclopentyl-2-[(1,1-dioxothiolan-3-yl)amino]-N-(5-hydroxy-2-adamantyl)pyrimidine-5-carboxamide,
4-cyclopentyl-N-(5-hydroxy-2-adamantyl)-2-[[(1S,2R)-2-hydroxycyclopentyl]amino]pyrimidine-5-carboxamide,
4-cyclopentyl-N-(5-hydroxy-2-adamantyl)-2-[[(2S)-tetrahydrofuran-2-yl]methylamino]pyrimidine-5-carboxamide,
4-cyclopentyl-N-(5-hydroxy-2-adamantyl)-2-[[(2R)-2-hydroxypropyl]amino]pyrimidine-5-carboxamide,
4-cyclopentyl-2-[[2-(dimethylamino)-2-oxo-ethyl]amino]-N-(5-hydroxy-2-adamantyl)pyrimidine-5-carboxamide,
4-cyclopentyl-N-(5-hydroxy-2-adamantyl)-2-(2-methoxyethylamino)pyrimidine-5-carboxamide,
4-cyclopentyl-N-(5-hydroxy-2-adamantyl)-2-[[(2R)-tetrahydrofuran-2-yl]methylamino]pyrimidine-5-carboxamide,
4-cyclopentyl-N-(5-hydroxy-2-adamantyl)-2-[[(2S)-2-hydroxypropyl]amino]pyrimidine-5-carboxamide,
4-cyclopentyl-N-(5-hydroxy-2-adamantyl)-2-[[(3S)-2-oxopyrrolidin-3-yl]amino]pyrimidine-5-carboxamide,
4-cyclopentyl-N-(5-hydroxy-2-adamantyl)-2-[[(3R)-5-oxopyrrolidin-3-yl]methylamino]pyrimidine-5-carboxamide,
2-[[(3R)-1-acetylpyrrolidin-3-yl]amino]-4-cyclopentyl-N-(5-hydroxy-2-adamantyl)pyrimidine-5-carboxamide,
4-cyclopentyl-N-(5-hydroxy-2-adamantyl)-2-[(1-methyl-5-oxo-pyrrolidin-3-yl)amino]pyrimidine-5-carboxamide,
4-cyclopentyl-2-[(1-ethyl-5-oxo-pyrrolidin-3-yl)amino]-N-(5-hydroxy-2-adamantyl)pyrimidine-5-carboxamide,
4-cyclopentyl-N-(5-hydroxy-2-adamantyl)-2-[[(2R)-1-methyl-5-oxo-pyrrolidin-2-yl]methylamino]pyrimidine-5-carboxamide,
4-cyclopentyl-N-(5-hydroxy-2-adamantyl)-2-[[(3S)-5-oxopyrrolidin-3-yl]methylamino]pyrimidine-5-carboxamide,
N-(5-hydroxy-2-adamantyl)-4-isopropyl-2-[[(3S)-tetrahydrofuran-3-yl]amino]pyrimidine-5-carboxamide,
N-(5-hydroxy-2-adamantyl)-4-isopropyl-2-[[(3R)-tetrahydrofuran-3-yl]amino]pyrimidine-5-carboxamide,
N-(5-hydroxy-2-adamantyl)-2-[[(3R)-oxolan-3-yl]amino]-4-(trifluoromethyl)pyrimidine-5-carboxamide,
N-(5-hydroxy-2-adamantyl)-2-[[(3S)-oxolan-3-yl]amino]-4-(trifluoromethyl)pyrimidine-5-carboxamide,
4-ethoxy-N-(5-hydroxy-2-adamantyl)-2-[[(3R)-tetrahydrofuran-3-yl]amino]pyrimidine-5-carboxamide,
4-ethoxy-N-(5-hydroxy-2-adamantyl)-2-[[(3S)-tetrahydrofuran-3-yl]amino]pyrimidine-5-carboxamide,
4-(cyclobutoxy)-N-(5-hydroxy-2-adamantyl)-2-[[(3S)-tetrahydrofuran-3-yl]amino]pyrimidine-5-carboxamide,
4-(cyclobutoxy)-N-(5-hydroxy-2-adamantyl)-2-[[(3R)-tetrahydrofuran-3-yl]amino]pyrimidine-5-carboxamide,
4-(cyclobutoxy)-N-(5-hydroxy-2-adamantyl)-2-(2-hydroxyethylamino)pyrimidine-5-carboxamide,
4-(cyclobutoxy)-N-(5-hydroxy-2-adamantyl)-2-(oxetan-3-ylamino)pyrimidine-5-carboxamide,
4-(cyclobutoxy)-N-(5-hydroxy-2-adamantyl)-2-[(2-hydroxy-1,1-dimethyl-ethyl)amino]pyrimidine-5-carboxamide,
4-(cyclobutoxy)-2-[(1,1-dioxothian-4-yl)amino]-N-(5-hydroxy-2-adamantyl)pyrimidine-5-carboxamide,
4-(cyclobutoxy)-N-(5-hydroxy-2-adamantyl)-2-(tetrahydropyran-4-ylamino)pyrimidine-5-carboxamide,
N-(5-hydroxy-2-adamantyl)-4-propoxy-2-[[(3R)-tetrahydrofuran-3-yl]amino]pyrimidine-5-carboxamide,
N-(5-hydroxy-2-adamantyl)-4-propoxy-2-[[(3S)-tetrahydrofuran-3-yl]amino]pyrimidine-5-carboxamide,
N-(5-hydroxy-2-adamantyl)-4-isopropoxy-2-[[(3S)-tetrahydrofuran-3-yl]amino]pyrimidine-5-carboxamide,
N-(5-hydroxy-2-adamantyl)-4-isopropoxy-2-[[(3R)-tetrahydrofuran-3-yl]amino]pyrimidine-5-carboxamide,
N-(5-hydroxy-2-adamantyl)-4-pyrrolidin-1-yl-2-[[(3R)-tetrahydrofuran-3-yl]amino]pyrimidine-5-carboxamide,
N-(5-hydroxy-2-adamantyl)-4-pyrrolidin-1-yl-2-[[(3S)-tetrahydro furan-3-yl]amino]pyrimidine-5-carboxamide,
N-(5-hydroxy-2-adamantyl)-4-pyrazol-1-yl-2-[[(3R)-tetrahydrofuran-3-yl]amino]pyrimidine-5-carboxamide,
N-(5-hydroxy-2-adamantyl)-4-pyrazol-1-yl-2-[[(3S)-tetrahydrofuran-3-yl]amino]pyrimidine-5-carboxamide,
4-Cyclopentyl-N-(5-hydroxy-2-adamantyl)-N-methyl-2-[[(3R)-oxolan-3-yl]amino]pyrimidine-5-carboxamide,
4-cyclopentyl-N-(5-hydroxy-2-adamantyl)-N-methyl-2-[[(3S)-oxolan-3-yl]amino]pyrimidine-5-carboxamide,
N-(5-carbamoyl-2-adamantyl)-4-cyclopentyl-2-[[(3S)-tetrahydrofuran-3-yl]amino]pyrimidine-5-carboxamide,
4-[[4-cyclopentyl-2-[[(3S)-tetrahydrofuran-3-yl]amino]pyrimidine-5-carbonyl]amino]adamantane-1-carboxylic acid, and
4-cyclopentyl-N-(5-methylsulfonyl-2-adamantyl)-2-[[(3S)-tetrahydrofuran-3-yl]amino]pyrimidine-5-carboxamide.

4. One or more compound(s) according to claim 2, selected from the group consisting of:
4-cyclobutyl-N-(5-hydroxy-2-adamantyl)-2-[[(3S)-tetrahydrofuran-3-yl]amino]pyrimidine-5-carboxamide,
4-cyclobutyl-N-(5-hydroxy-2-adamantyl)-2-[[(3R)-tetrahydrofuran-3-yl]amino]pyrimidine-5-carboxamide,
2-[(1-acetyl-4-piperidyl)amino]-4-cyclopentyl-N-(5-hydroxy-2-adamantyl)pyrimidine-5-carboxamide,
4-cyclopentyl-N-(5-hydroxy-2-adamantyl)-2-[[(3R)-6-oxo-3-piperidyl]amino]pyrimidine-5-carboxamide,
4-cyclopentyl-2-[(1,1-dioxothiolan-3-yl)amino]-N-(5-hydroxy-2-adamantyl)pyrimidine-5-carboxamide,
4-cyclopentyl-N-(5-hydroxy-2-adamantyl)-2-[[(1S,2R)-2-hydroxycyclopentyl]amino]pyrimidine-5-carboxamide,
4-cyclopentyl-N-(5-hydroxy-2-adamantyl)-2-[[(2S)-tetrahydrofuran-2-yl]methylamino]pyrimidine-5-carboxamide,
4-cyclopentyl-N-(5-hydroxy-2-adamantyl)-2-[[(2R)-2-hydroxypropyl]amino]pyrimidine-5-carboxamide,
4-cyclopentyl-2-[[2-(dimethylamino)-2-oxo-ethyl]amino]-N-(5-hydroxy-2-adamantyl)pyrimidine-5-carboxamide,
4-cyclopentyl-N-(5-hydroxy-2-adamantyl)-2-(2-methoxyethylamino)pyrimidine-5-carboxamide,
4-cyclopentyl-N-(5-hydroxy-2-adamantyl)-2-[[(2R)-tetrahydrofuran-2-yl]methylamino]pyrimidine-5-carboxamide,
4-cyclopentyl-N-(5-hydroxy-2-adamantyl)-2-[[(2S)-2-hydroxypropyl]amino]pyrimidine-5-carboxamide,
4-cyclopentyl-N-(5-hydroxy-2-adamantyl)-2-[[(3S)-2-oxopyrrolidin-3-yl]amino]pyrimidine-5-carboxamide,
4-cyclopentyl-N-(5-hydroxy-2-adamantyl)-2-[[(3R)-5-oxopyrrolidin-3-yl]methylamino]pyrimidine-5-carboxamide,
2-[[(3R)-1-acetylpyrrolidin-3-yl]amino]-4-cyclopentyl-N-(5-hydroxy-2-adamantyl)pyrimidine-5-carboxamide,
4-cyclopentyl-N-(5-hydroxy-2-adamantyl)-2-[(1-methyl-5-oxo-pyrrolidin-3-yl)amino]pyrimidine-5-carboxamide,
4-cyclopentyl-2-[(1-ethyl-5-oxo-pyrrolidin-3-yl)amino]-N-(5-hydroxy-2-adamantyl)pyrimidine-5-carboxamide,
4-cyclopentyl-N-(5-hydroxy-2-adamantyl)-2-[[(2R)-1-methyl-5-oxo-pyrrolidin-2-yl]methylamino]pyrimidine-5-carboxamide,
4-cyclopentyl-N-(5-hydroxy-2-adamantyl)-2-[[(3S)-5-oxopyrrolidin-3-yl]methylamino]pyrimidine-5-carboxamide,
N-(5-hydroxy-2-adamantyl)-4-isopropyl-2-[[(3S)-tetrahydrofuran-3-yl]amino]pyrimidine-5-carboxamide,
N-(5-hydroxy-2-adamantyl)-4-isopropyl-2-[[(3R)-tetrahydrofuran-3-yl]amino]pyrimidine-5-carboxamide,
N-(5-hydroxy-2-adamantyl)-2-[[(3R)-oxolan-3-yl]amino]-4-(trifluoromethyl)pyrimidine-5-carboxamide,
N-(5-hydroxy-2-adamantyl)-2-[[(3S)-oxolan-3-yl]amino]-4-(trifluoromethyl)pyrimidine-5-carboxamide,
4-ethoxy-N-(5-hydroxy-2-adamantyl)-2-[[(3R)-tetrahydrofuran-3-yl]amino]pyrimidine-5-carboxamide,
4-ethoxy-N-(5-hydroxy-2-adamantyl)-2-[[(3S)-tetrahydrofuran-3-yl]amino]pyrimidine-5-carboxamide,
4-(cyclobutoxy)-N-(5-hydroxy-2-adamantyl)-2-[[(3S)-tetrahydrofuran-3-yl]amino]pyrimidine-5-carboxamide,
4-(cyclobutoxy)-N-(5-hydroxy-2-adamantyl)-2-[[(3R)-tetrahydrofuran-3-yl]amino]pyrimidine-5-carboxamide,
4-(cyclobutoxy)-N-(5-hydroxy-2-adamantyl)-2-(2-hydroxyethylamino)pyrimidine-5-carboxamide,
4-(cyclobutoxy)-N-(5-hydroxy-2-adamantyl)-2-(oxetan-3-ylamino)pyrimidine-5-carboxamide,
4-(cyclobutoxy)-N-(5-hydroxy-2-adamantyl)-2-[(2-hydroxy-1,1-dimethyl-ethyl)amino]pyrimidine-5-carboxamide,
4-(cyclobutoxy)-2-[(1,1-dioxothian-4-yl)amino]-N-(5-hydroxy-2-adamantyl)pyrimidine-5-carboxamide,
4-(cyclobutoxy)-N-(5-hydroxy-2-adamantyl)-2-(tetrahydropyran-4-ylamino)pyrimidine-5-carboxamide,
N-(5-hydroxy-2-adamantyl)-4-propoxy-2-[[(3R)-tetrahydrofuran-3-yl]amino]pyrimidine-5-carboxamide,
N-(5-hydroxy-2-adamantyl)-4-propoxy-2-[[(3S)-tetrahydrofuran-3-yl]amino]pyrimidine-5-carboxamide,
N-(5-hydroxy-2-adamantyl)-4-isopropoxy-2-[[(3S)-tetrahydrofuran-3-yl]amino]pyrimidine-5-carboxamide,
N-(5-hydroxy-2-adamantyl)-4-isopropoxy-2-[[(3R)-tetrahydrofuran-3-yl]amino]pyrimidine-5-carboxamide,
4-Cyclopentyl-N-(5-hydroxy-2-adamantyl)-N-methyl-2-[[(3R)-oxolan-3-yl]amino]pyrimidine-5-carboxamide,
4-cyclopentyl-N-(5-hydroxy-2-adamantyl)-N-methyl-2-[[(3S)-oxolan-3-yl]amino]pyrimidine-5-carboxamide, and
N-(5-carbamoyl-2-adamantyl)-4-cyclopentyl-2-[[(3S)-tetrahydrofuran-3-yl]amino]pyrimidine-5-carboxamide.

5. One or more compound(s) according to claim 1, selected from the group consisting of:
4-cyclobutyl-N-(5-hydroxy-2-adamantyl)-2-[[(3R)-tetrahydrofuran-3-yl]amino]pyrimidine-5-carboxamide,
2-[(1-acetyl-4-piperidyl)amino]-4-cyclopentyl-N-(5-hydroxy-2-adamantyl)pyrimidine-5-carboxamide,
4-cyclopentyl-N-(5-hydroxy-2-adamantyl)-2-[[(3R)-6-oxo-3-piperidyl]amino]pyrimidine-5-carboxamide,
4-cyclopentyl-2-[(1,1-dioxothiolan-3-yl)amino]-N-(5-hydroxy-2-adamantyl)pyrimidine-5-carboxamide,
4-cyclopentyl-N-(5-hydroxy-2-adamantyl)-2-[[(1S,2R)-2-hydroxycyclopentyl]amino]pyrimidine-5-carboxamide,
4-cyclopentyl-N-(5-hydroxy-2-adamantyl)-2-[[(2S)-tetrahydrofuran-2-yl]methylamino]pyrimidine-5-carboxamide,
4-cyclopentyl-N-(5-hydroxy-2-adamantyl)-2-[[(2R)-2-hydroxypropyl]amino]pyrimidine-5-carboxamide,
4-cyclopentyl-N-(5-hydroxy-2-adamantyl)-2-(2-methoxyethylamino)pyrimidine-5-carboxamide,
4-cyclopentyl-N-(5-hydroxy-2-adamantyl)-2-[[(2R)-tetrahydrofuran-2-yl]methylamino]pyrimidine-5-carboxamide,
4-cyclopentyl-N-(5-hydroxy-2-adamantyl)-2-[[(2S)-2-hydroxypropyl]amino]pyrimidine-5-carboxamide,
2-[[(3R)-1-acetylpyrrolidin-3-yl]amino]-4-cyclopentyl-N-(5-hydroxy-2-adamantyl)pyrimidine-5-carboxamide,
4-cyclopentyl-N-(5-hydroxy-2-adamantyl)-2-[(1-methyl-5-oxo-pyrrolidin-3-yl)amino]pyrimidine-5-carboxamide,
4-cyclopentyl-2-[(1-ethyl-5-oxo-pyrrolidin-3-yl)amino]-N-(5-hydroxy-2-adamantyl)pyrimidine-5-carboxamide,
4-cyclopentyl-N-(5-hydroxy-2-adamantyl)-2-[[(2R)-1-methyl-5-oxo-pyrrolidin-2-yl]methylamino]pyrimidine-5-carboxamide,
4-cyclopentyl-N-(5-hydroxy-2-adamantyl)-2-[[(3S)-5-oxopyrrolidin-3-yl]methylamino]pyrimidine-5-carboxamide,
N-(5-hydroxy-2-adamantyl)-2-[[(3R)-oxolan-3-yl]amino]-4-(trifluoromethyl)pyrimidine-5-carboxamide,
4-(cyclobutoxy)-N-(5-hydroxy-2-adamantyl)-2-[[(3S)-tetrahydrofuran-3-yl]amino]pyrimidine-5-carboxamide,
4-(cyclobutoxy)-N-(5-hydroxy-2-adamantyl)-2-[[(3R)-tetrahydrofuran-3-yl]amino]pyrimidine-5-carboxamide,
4-(cyclobutoxy)-N-(5-hydroxy-2-adamantyl)-2-(2-hydroxyethylamino)pyrimidine-5-carboxamide,
4-(cyclobutoxy)-N-(5-hydroxy-2-adamantyl)-2-(oxetan-3-ylamino)pyrimidine-5-carboxamide,
N-(5-hydroxy-2-adamantyl)-4-propoxy-2-[[(3R)-tetrahydrofuran-3-yl]amino]pyrimidine-5-carboxamide,
N-(5-hydroxy-2-adamantyl)-4-propoxy-2-[[(3S)-tetrahydrofuran-3-yl]amino]pyrimidine-5-carboxamide,
N-(5-hydroxy-2-adamantyl)-4-isopropoxy-2-[[(3S)-tetrahydrofuran-3-yl]amino]pyrimidine-5-carboxamide,
N-(5-hydroxy-2-adamantyl)-4-isopropoxy-2-[[(3R)-tetrahydrofuran-3-yl]amino]pyrimidine-5-carboxamide, and
N-(5-carbamoyl-2-adamantyl)-4-cyclopentyl-2-[[(3S)-tetrahydrofuran-3-yl]amino]pyrimidine-5-carboxamide.

6. A pharmaceutical composition, which comprises a compound of formula (I), or a pharmaceutically acceptable salt thereof, as claimed in claim 1 in association with a pharmaceutically-acceptable diluent or carrier.

7. A pharmaceutical composition, which comprises a compound of formula (I), or a pharmaceutically acceptable salt thereof, as claimed in claim 3 in association with a pharmaceutically-acceptable diluent or carrier.

8. A method of producing a 11βHSD1-inhibitory effect in a warm-blooded animal comprising administering to the animal a compound as defined in claim 1, or a pharmaceutically-acceptable salt thereof.

9. A method of producing a 11βHSD1-inhibitory effect in a warm-blooded animal comprising administering to the animal a compound as defined in claim 3, or a pharmaceutically-acceptable salt thereof.
